(19) United States
(12) Patent Application Publication
(10) Pub. No.: US 2023/0365622 A1
(43) Pub. Date: Nov. 16, 2023

(54) PROTEIN RECEPTACLE, POLYNUCLEOTIDE, VECTOR, EXPRESSION CASSETTE, CELL, METHOD FOR PRODUCING THE RECEPTACLE, METHOD OF IDENTIFYING PATHOGENS OR DIAGNOSING DISEASES, USE OF THE RECEPTACLE AND DIAGNOSTIC KIT

(71) Applicant: FUNDÃÇAO OSWALDO CRUZ, Rio de Janeiro, RJ (BR)

(72) Inventors: David William PROVANCE, Jr., Rio de Janeiro - RJ (BR); Andressa da Matta DURANS, Rio de Janeiro - RJ (BR); Paloma Napleão PÊGO, Rio de Janeiro - RJ (BR); Salvatore Giovanni DE SIMONE, Rio de Janeiro - RJ (BR)

(73) Assignee: FUNDÃÇAO OSWALDO CRUZ, Rio de Janeiro, RJ (BR)

(21) Appl. No.: 17/638,108

(22) PCT Filed: Aug. 27, 2020
(86) PCT No.: PCT/BR2020/050341

§ 371 Date: Feb. 24, 2022

(30) Foreign Application Priority Data
Aug. 27, 2019 (BR) BR1020190177926

(51) Int. Cl. C07K 1/04; G01N 33/569

(52) U.S. Cl. CPC C07K 1/042; G01N 33/569; G01N 2333/44; G01N 2333/175; G01N 2333/181; G01N 2333/165

(57) ABSTRACT

The present invention relates to a protein receptacle capable of receiving several exogenous polyamino acid sequences, concomitantly, for expression in various systems and for different uses. The present invention relates to polynucleotides capable of generating the aforementioned protein receptacle. The present invention also relates to vector and expression cassette comprising the aforementioned polynucleotide. The present invention further relates to the cell comprising the aforementioned expression vector or cassette. The present invention further relates to a method for producing said protein receptacle and for pathogen identification or disease diagnosis in vitro. The present invention further relates to the use of said protein receptacle and kit comprising said protein receptacle for diagnostic purposes or as vaccine compositions.

### FIELD OF INVENTION

The present invention falls within the field of application of Chemistry, Pharmacy, Medicine, Biotechnology and, more specifically, in the area of preparations for biomedical purposes. The present invention relates to a protein receptacle capable of receiving several exogenous polyamino acid sequences, concomitantly, for expression in various systems and for different uses. The present invention relates to polynucleotides capable of generating protein receptacles mentioned above. The present invention also relates to vector and expression cassette comprising the aforementioned polynucleotide(s). The present invention further relates to a cell comprising the aforementioned vector or expression cassette. The present invention further relates to a method for producing said protein receptacle and for pathogen identification or disease diagnosis in vitro. The present invention further relates to the use of said protein receptacle and kit comprising said protein receptacle for diagnostic purposes or as vaccine compositions.

### BACKGROUND OF THE INVENTION

This document has several references throughout the text, which are indicated within parentheses. The information published in these references is included here in order to better describe the state of the art to which the present invention belongs.

The green fluorescent protein (GFP), produced by a cnidarian, the jellyfish Aequorea victoria emits high fluorescence in the green zone of the visible spectrum (Prasher et al., Gene 15, 111 (2): 229-33, 1992 (1]) and, classically, has been used as a marker of gene expression and localization, and, thus, known as reporter protein. After the first observation that the GFP protein could emit fluorescence, several uses were described for the protein.

The use of GFP as a reporter protein has been patented for different uses and in different systems. Patent application US2018298058 uses GFP as a protein production and purification system. Patent application CN108303539 uses GFP as an input for cancer detection testing; application CN108220313 presents a high-throughput GFP fusion and expression method. Application CN108192904 claims a GFP fusion protein capable of inserting itself into biological membranes; application CN107703219 already emphasizes the use of GFP in the metabolomic study of mesenchymal cells. Still, patent application US2018016310 presents variations of “superfolder” fusion GFP. There are several patents, such as these examples: U.S. Pat. Nos. 6,054,321, 6,096,865, 6,027,881, 6,025,485 that have mutated GFPs to increase fluorescence expression or modify fluorescence wavelength peaks. Thus, it is clear that the GFP protein, since its description, has been used and patented for various uses as a reporter protein.

Reporter molecules are often used in biological systems to monitor gene expression. The GFP protein brought great innovation in this scenario by dispensing with the use of any substrate or cofactor, that is, it does not require the addition of any other reagent in order to be visualized, as occurs for most other reporter proteins. Another advantage presented by GFP is its ability to show autofluorescence, not requiring fluorescent markers that, for various causes, do not have the appropriate sensitivity or specificity for its use.

In light of this characteristic, its self-production of detectable green fluorescence, GFP has been widely used to study gene expression and protein localization, and is considered one of the most promising reporter proteins in the literature.

In its use as a reporter protein, the gene encoding GFP can be employed in the production of fusion proteins, i.e. a particular gene of interest is fused to the gene encoding GFP. The fused gene cassette can be inserted into a living system, allowing expression of the fused genes and monitoring of the intracellular localization of that protein of interest (Santos-Beneit & Errington, Archives Microbiology, 199 (6): 875-880, 2017; Belardinelli & Jackson, Tuberculosis (Edinb), 105: 13-17, 2017; Wakabayashi et al, International Journal Food Microbiology, 19, 291: 144-150, 2018; Cal et al, Viruses, 20; 10 (11), 2018).

The GFP protein has also been useful as a framework for peptide presentation or even peptide libraries, both in yeast and mammalian cell systems (Kamb et al., Proc. Natl. Acad Sci. USA, 95: 7508-7513, 1998; WO 2004005322). The GFP protein, as a framework protein for the presentation of random peptides, can be used to define the characteristics of a peptide library.

Advances in the development of new variations of GFP seek to achieve improvements in the properties of the protein in order to produce new reagents useful for a wide range of research purposes. New versions of GFP have been developed, via mutations, containing DNA sequences optimized for increased production in human cellular systems, i.e., humanized GFP proteins (Cormack, et al., Gene 173, 33-38, 1996; Haas, et al., Current Biology 6, 315-324, 1996; Yang, et al., Nucleic Acids Research 24, 4592-4593, 1996).

In one of these versions, the enhanced green fluorescent protein, the “enhanced green fluorescent protein” (eGFP) was described (Heim & Cubitt, Nature, 373, pp. 663-664, 1995).

A GFP, encoded by the gfp10 gene originating from a cnidarian, the jellyfish Aequorea victoria, is a protein of 238 amino acids. The protein has the ability to absorb blue light (with a main excitation peak at 395 nm) and emit green light, from a chromophore in the center of the protein (main emission peak at 509 nm) (Morin & Hastings, Journal Cell Physiology, 77(3): 313-8, 1971; Prasher et al, Gene 15, 111 (2): 229-33, 1992). The chromophore is composed of six peptides and starts at amino acid 64, and is derived from the primary amino acid sequence by cyclization and oxidation of serine, tyrosine and glycine (at positions 65, 66 and 67) (Shimomura, 104 (2), 1979; Cody et al., Biochemistry, 32 (5): 1212-8, 1993). The light emitted by GFP is independent of the cell biological species where it is expressed and does not require any type of substrate, cofactors or additional gene products from A. victoria (Chalfie et al., Science, 263 (5148): 802-5, 1994). This property of GFP allows its fluorescence to be detected in living cells other than A. victoria, provided that it can be processed in the cell's protein expression system (Ormo et al, Science 273: 1392-1395, 1996; Yang et al, Nature Biotech 14: 1246-1251, 1996).

The basic structure of a GFP consists of eleven antiparallel pleated beta chains, which are intertwined to form a tertiary structure in the shape of a beta barrel. Each chain is connected to the next by a domain of handle that projects to the top and bottom surface of the barrel, interacting with the environment. By convention, each string e loop can be identified by a number in order to better describe the protein.

Targeted mutation experiments have shown that several biochemical properties of the GFP protein arise from this barrel structure. And therefore, amino acid changes in the primary structure are responsible for accelerating protein folding, reducing the aggregation of translation products, and increasing the stability of the protein in solution.

A particular loop moves into the cavity of the protein barrel, forming an alpha-helix that is responsible for the fluorescent properties of the protein (Crone et al, GFP-Based Biosensors, InTech, 2013). Some interventions in the protein structure can interfere with the ability to emit fluorescence. Mutations in certain amino acids can change from the intensity of fluorescence emission to the wavelength, changing the emitted color. Mutation of Tyr66, an internal residue participating in the fluorescent chromophore, can generate a large number of fluorescent protein variants with the structure of the altered chromophore or the surrounding environment. These changes interfere with the absorption and emission of light at different wavelengths, producing a wide range of distinct emitted colors (Heim & Tsien, Current Biology, 6 (2): 178-82, 1996).

Changes in pH can also interfere with fluorescence intensity. At physiological pH, GFP exhibits maximum absorption at 395 nm, while at 475 nm it absorbs less light. However, increasing the pH to about 12.0 causes the absorption maximum to occur in the 475 nm range, and at 395 nm it has decreased absorption (Ward et al, Photochemistry and Photobiology, 35(6): 803-808, 1982).

The compact structure of the protein core allows GFP to be highly stable even under adverse conditions, such as treatment by proteases, making the protein extremely useful as a reporter protein in general.

There are different versions of the GFP, always seeking to improve the protein by adding new functionality or removing some limitation. The eGFP presents itself as an enhancement that allows for greater flexibility of the protein in the face of modifications to the F64L and S65T amino acids (Heim et al, Nature 373: 663-664, 1995; Li et al, Journal Biology Chemistry, 272 (45): 28545-9, 1997). This enhancement allows GFP to achieve both its expected three-dimensional shape and its ability to express fluorescence, even when harboring heterologous sequences in its protein sequence (Pedelacq et al, Nat Biotechnology, 24 (1): 79-88, 2006).

GFAb, is a version of the modified protein that accepts the exogenous sequences in two loop domains. In its development, several rounds of direct evolution were required to select three mutated protein clones that supported the insertion of exogenous peptides into the two proximal regions, namely Glu-172-Asp-173 and Asp-102-Asp-103. The authors using unmutated proteins proved that the insertion of two exogenous peptides prevented GFP fluorescence production and protein expression on the yeast cell surface. Simultaneous insertion, in only two regions, was possible after a series of mutations and selections, but still resulting in great loss of the inherent activities of GFP, sometimes making its production or expression of the insert impossible (Pavoor et al, PNAS 106 (29): 11895-11900, 2009).

Mutants circularly permuted to the N- and C-terminals have also demonstrated that eGFP is amenable to manipulation in the coding sequence without compromising the structural aspects of the protein core (Topell et al., FEBS Letters 457(2): 283-289, 1999). However, analysis of 20 circularly permuted protein variants demonstrated the proteins' low tolerance to insertion of a new terminus and, for the most part, that they lose the ability to form the chromophore. This fact indicates that manipulation of the protein sequence can drastically interfere with its characteristics or even its cellular expression.

Several attempts have been made to simultaneously insert multiple epitopes into the loop regions of GFP with the goal of achieving use of the protein for specific binding reactions to a target. However, all these efforts have shown limited success in light of the structural sensitivity of GFP and its chromophore.

Other mutant proteins of the GFP protein show improved versions that emit other types of fluorescent light spectrum. For example, Heim et al (Proc Natl Acad Sci USA, 91 (26): 12501-4, 1994) described a mutant protein that emits blue fluorescence by containing a histidine instead of a tyrosine at amino acid 66. Heim et al (Nature, 373 (6516): 663-4, 1995) subsequently also described a mutant GFP protein, by substitution of a serine for a threonine at amino acid 65, which has a spectrum very similar to that obtained from Renilla reniformis, which has a 10-fold higher extinction coefficient per monomer than the wavelength peak of the native GFP from Aequorea. Other patent documents describe mutant GFP proteins showing light emission spectra other than green, such as blue, red (U.S. Pat. No. 5,625,048, WO 2004005322).

Also, other GFP mutant proteins have excitation spectra optimized for use specifically in certain argon laser flow cytometer (FACS) equipment (U.S. Pat. No. 5,804,387). There is still a description of mutant GFP proteins modified to be better expressed in plant cell systems (WO1996027675). The patent document U.S. Pat. No. 5,968,750 presents a humanized GFP that has been adapted to be expressed in mammalian cells, including human. Humanized GFP incorporates preferential codons for reading into human cell gene expression systems.

In the state of the art, it is clear that GFP is capable of harboring genes at its 5′ or 3′ ends without interfering with expression, three-dimensional tangling, and fluorescence production, as can be seen in the patent documents listed above. Additionally, GFP has been used as a carrier for peptide display or even peptide libraries in vivo. In the case of peptide libraries, GFP can assist in the presentation of random peptides, and thus in defining the characteristics of the peptide library (Kamb et al., Proc. Natl. Acad. Sci. USA, 95:7508-7513, 1998; WO 2004005322).

For example, Abedi et al (1998, Nucleic Acids Res. 26: 623-300) inserted peptides into GFP proteins of Aequorea victoria in regions of exposed loops and demonstrated that GFP molecules retain autofluorescence when expressed in yeast and Escherichia coli. The authors further demonstrated that the fluorescence of a GFP frame can be used to monitor peptide diversity, as well as the presence or expression of a given peptide in a given cell. However, the fluorescence rate of the GFP framework molecules is relatively low compared to natural GFP. Kamb and Abedi (U.S. Pat. No. 6,025,485) prepared libraries of GFP arrays from enhanced green fluorescent protein (eGFP) in order to amplify fluorescence intensity.

Additionally, Peele et al (Chem. & Bio. 8: 521-534, 2001) tested peptide libraries using eGFP as a framework with different structural biases in mammalian cells. Anderson et al further amplified the fluorescence intensity by inserting peptides into the GFP loops with tetraglycine ligands (US20010003650). Happe et al described a humanized GFP that can be expressed in large quantities in mammalian cell systems, tolerates peptide insertions, and preserves autofluorescence (WO 2004005322).

However, there is still a need in the technological field for GFP molecule frameworks that not only display fluorescence at appropriate intensities, but that can also be expressed at high levels in cellular systems.

There is variability in tolerance, among GFP molecules, for peptide presentation while retaining autofluorescence. Thus, there is a need in the state of the art to develop GFPs that can be expressed at high levels and tolerate insertions, while preserving autofluorescence.

In addition to the ability to support gene expression at the ends, GFP may allow the insertion of epitopes into the surface loops of the molecule exposed to the medium. Several attempts have been made to simultaneously insert multiple peptides into the loop regions of GFP that could allow the protein to be used for target-specific binding reactions. However, all of these efforts have had limited success in light of the structural sensitivity of GFP and its chromophore.

Pavoor and colleagues undertook efforts to develop a protein modified to accept exogenous sequences in two loop domains. In its development, several rounds of direct evolution were necessary to select three mutated protein clones that supported the insertion of exogenous peptides in the two proximal regions, namely, Glu-172-Asp-173 and Asp-102-Asp-103. The authors using unmutated proteins proved that the insertion of two exogenous peptides prevented GFP fluorescence production and protein expression on the yeast cell surface. Simultaneous insertion, in only two regions, was possible after a series of mutations and selections, but still resulted in a great loss of the inherent activities of GFP, sometimes making its production or expression of the insert impossible (Pavoor et al, PNAS 106 (29): 11895-11900, 2009).

Abedi et al (Nucleic Acids Research 26(2): 623-30, 1998) showed 10 positions of the protein, in loop regions, of which 8 positions between-β-sheets, for peptide expression. The chimeric protein would be useful for experiments requiring intracellular expression, and therefore fluorescence uninterruptedness would be a limiting factor. In this study, only three chimeric proteins (those with insertion sites at amino acids 157-158 172-173 and 194-195) showed fluorescence (dimmed to a quarter of the original); and only two insertion sites (studied separately) could harbor peptides without loss of fluorescence. The authors of the paper further concluded that “it is curious how GFP is so sensitive to structural perturbations even if it is in β-sheets.”

Li et al (Photochemistry and Photobiology, 84(1): 111-9, 2008) present a study of the chimeric protein (red fluorescent protein—RFP), pointing out in this protein six genetically distinct sites located in three different loops where sequences of five residues can be inserted without interfering with the ability of the protein to be fluorescent. However, the authors have not demonstrated the concomitant use of these sites for insertion of different peptides.

Patent application WO02090535 presents a fluorescent GFP with non-simultaneous peptide insertions into 5 different loops of the protein. The patent application, in its descriptive report, indicates the possibility of inserting peptides in more than one loop of the protein at the same time, increasing the complexity of the library and allowing presentation on the same face of the protein. However, the patent application does not prove this possibility, since it only presents insertion assays of peptides, one at a time, in 5 different protein loops. It is worth noting that the text further emphasizes that loops 1 and 5 do not present themselves as good insertion sites, because peptide insertion at these sites prevented protein expression. Still other patent documents present variations of GFP aiming at the expression of peptides in the protein's loops, however, these studies do not prove the feasibility of simultaneous expression of more than 4 peptides in different insertion sites in the GFP protein loops without loss of any of its essential characteristics (WO02090535, US2003224412, WO200134824).

### SUMMARY OF THE INVENTION

To solve the problems mentioned above, the present invention will provide significant advantages, since the receptacle proteins can express a large number of different polyamino acid sequences, being characterized as multivalent receptacle proteins, expanding their use for vaccine composition purposes, an input for research and technological development or disease diagnosis. There is a real need in the state of the art to develop receptacle proteins that not only exhibit adequate fluorescence intensities, but can also be expressed in large quantities in production cell systems and, furthermore, tolerate the concomitant presentation of multiple exogenous polyamino acid sequences while still exhibiting detectable autofluorescence.

In one aspect, the present invention relates to a protein receptacle capable of presenting several exogenous polyamino acid sequences concurrently at more than four different sites on the receptacle protein.

In another aspect, the invention relates to polynucleotides capable of generating the aforementioned protein receptacle.

In another aspect, the invention relates to a vector comprising the aforementioned polynucleotide.

In another aspect, the invention relates to expression cassette comprising the aforementioned polynucleotide.

In another aspect, the invention relates to a method for producing said protein receptacle and for pathogen identification or disease diagnosis in vitro.

In another aspect, the present invention relates to the use of said protein receptacle for diagnostic purposes or as vaccine compositions.

In another aspect, the present invention relates to kit comprising said protein receptacle for diagnostic purposes or as vaccine compositions.

### BRIEF DESCRIPTION OF THE FIGURES

FIG. 1—Purification of PlatCruzi protein by affinity chromatography. (A) Elution profile of the receptacle protein on a nickel column using a Äktapurifier liquid chromatography system. (B) Analysis by polyacrylamide gel electrophoresis (SDS PAGE) of the collected 13-26 eluates. Elution was performed with buffer B and in ascending order. PM—Molecular Weight Marker.

FIG. 2—Reactivity of PlatCruzi, by ELISA, with sera from Trypanosoma cruzi International Standard Biological References provided by WHO. (A) Pool of patient sera recognizing TcI strains, called IS 09/188. (B) Pool of patient sera recognizing TcII strains, called IS 09/186.

FIG. 3—Determination of antibody titer of sera from patients with chronic Chagas disease using PlatCruzi receptacle protein as antigen. Sera were provided by the LACENS and a concentration of 500 ng/well and serum dilution 1:50-1:1000 were used in ELISAS

FIG. 4—Performance of the PlatCruzi antigen by ELISA against serum from patients with different diseases. PlatCruzi antigen was used at a concentration of 500 ng/well and sera diluted 1:250.

FIG. 5—Detection of rabies virus-specific epitopes by rabbit anti-RxRabies2 serum. Rabbit antibodies immunized with RxRabies2 were purified by RxRabies2 affinity chromatography and used as primary antibody, by Western blot, to detect RxRabies2 in a crude (*; column 2) or semi-purified (†) extract at two different concentrations of 1× and 0.5× (columns 4 and 6, respectively). Negative controls: Rx receptacle protein (column 3) and PlatCruzi (in two concentrations: 1× and 0.5× in columns 5 and 7, respectively).

FIG. 6—Analysis of RxHoIgG3 protein by polyacrylamide gel electrophoresis (SDS PAGE). A, soluble extract of E. coli not producing RxHoIgG3. B, Aqueous insoluble fraction of RxHoIgG3-producing bacteria. C, Soluble fraction of RxHoIgG3-producing bacteria. The arrows indicate the position of the RxHoIgG3 protein.

FIG. 7—Detection of IgM anti-RxOro antibodies by ELISA. C−: negative control (serum from patient without Oropouche virus infection); C+: standard positive serum for Oropouche virus infection. Patients: suspected cases of Oropouche virus infection. Oro+: positive for Oropouche infection (detection of IgM anti-RxOro antibodies). Oro− (negative control): No IgM reaction for RxOro. Protein concentration: 0.288 μg/μL. Cutoff: 0.0613.

FIG. 8—Polyacrylamide gel electrophoresis (SDS-PAGE) representing the production of the PlatCruzi, RxMayaro_IgG and RxMayaro_IgM proteins. Vertical columns 1 to 8, indicate: 1) molecular weight; 2) total bacterial extract without induction of recombinant protein; 3) total bacterial extract after induction of PlatCruzi production; 4) total bacterial extract after induction of RxMayaro_IgG production; 5) total bacterial extract after induction of RxMayaro_IgM production; 6) insoluble bacterial proteins after induction of PlatCruzi production; 7) insoluble bacterial proteins after induction of RxMayaro_IgG production; 8) insoluble bacterial proteins after induction of RxMayaro_IgM production. The arrows point to the bands representing PlatCruzi (columns 3 and 6), RxMayaro_IgG (columns 4 and 7) and RxMayaro_IgM (columns 5 and 8).

FIG. 9—Reactivity of sera from Mayaro virus positive patients (S MAY) and healthy individuals (S N), by ELISA, using the RxMayaro_IgG protein. Revealing was performed with anti-IgG immunoglobulin conjugated to alkaline phosphatase enzyme (Cutoff=0.0210).

FIG. 10—Reactivity of sera from individuals considered healthy (S N) and positive for Mayaro virus (S MAY), by ELISA, using the RxMayaro_IgM protein. Revealing was performed with anti-IgM immunoglobulin conjugated to alkaline phosphatase enzyme. (Cut off=0.0547).

FIG. 11—Polyacrylamide gel electrophoresis (SDS-PAGE) showing the yield of insoluble (I) and soluble (S) proteins from PlatCruzi, TxCruzi, RxPtx, TxNeuza, and RxYFIgG. Columns 1 through 10 contain: 1) insoluble proteins of bacteria induced to produce PlatCruzi; 2) soluble proteins of bacteria induced to produce PlatCruzi; 3) insoluble proteins of bacteria induced to produce TxCruzi; 4) soluble proteins of bacteria induced to produce TxCruzi; 5) insoluble proteins of bacteria induced to produce RxPtx; 6) soluble proteins of bacteria induced to produce RxPtx; 7) insoluble proteins of bacteria induced to produce TxNeuza; 8) soluble proteins of bacteria induced to produce TxNeuza; 9) insoluble proteins of bacteria induced to produce RxYFIgG; 10) soluble proteins of bacteria induced to produce RxYFIgG. The arrows point to the bands representing PlatCruzi (columns 1 and 2), TxCruzi (columns 3 and 4), RxPtx (columns 5 and 6), TxNeuza (columns 7 and 8) and RxYFIgG (columns 9 and 10).

FIG. 12—Pictorial cellulose membrane with polyamino acid of SARS-CoV-2 reacting with IgM antibodies from Covid-19 positive patient sera, as spots of various shades of gray within regions enclosed by a grid in the form of a checkerboard. Each square encompasses a reacting spot of the cellulose membrane region in which a distinct polypeptide sequence has been synthesized in linear form covalently bound to the membrane surface. The relationship between the physical position in the membrane and the sequence of the polyamino acid is listed in Table 18. The combined polyamino acid sequences represent the encoded sequence of the spike protein SARS-CoV-2 (S1: aa 1-1273, A7-K19), protein ORF3a (OF3: aa 1-275, K22-N2), membrane glycoprotein (M: aa 1-222, N5-023); ORF6 (OF6: aa 1-61, P2-P12); ORF7 protein (OF7: aa1-121, P15-Q13), ORF8 protein (OF8: aa 1-121, Q16-R17), nucleocapsid protein (N: aa1 419, R20-V17), envelope protein (E: aa 1-75, W1-W13), ORF10 protein regions (OF10: aa 1-38, W15-W20). Each polyamino acid has a length of 15 amino acids and an adjacent, continuous overlap of 10 amino acids.

FIG. 13—Pictorial cellulose polyamino acid membrane of SARS-CoV-2 reacted with IgG antibodies from Covid19 patient sera, as spots of various shades of gray visualized within regions bounded in the form of a grid. Each square encompasses a reacting spot of the cellulose membrane region in which a distinct polypeptide sequence has been synthesized in linear form covalently bound to the membrane surface. The relationship between the physical positions in the membrane and the sequences of the polyamino acids is listed in Table 19. The combined polyamino acid sequences represent the encoded sequence of the SARS-CoV-2 ORF3a protein (ORF3: aa 1-275, A7-C11), membrane glycoprotein (G: aa 1-61, C14-E8); ORF6 protein (ORF6: aa 1-61, E11-E21); ORF7 protein (OF7: aa1-121, E24-F22), ORF8 protein (ORF8: aa 1-121, G1-G23), spike protein (S: aa 1-1273, H1-R13), nucleocapsid protein (N: aa1-419, R16-V1), envelope protein (E: aa 1-75, W1-W13), ORF10 (ORF10: aa 1-38, W15-W20). Each polyamino acid has a length of 15 amino acids and an adjacent, continuous overlap of 10 amino acids.

FIG. 14—Pictorial cellulose membrane with polyamino acid of SARS-CoV-2 reacting with IgA antibodies, from Covid19 patient serum as spots of various shades of gray visualized within delimited regions in the form of a grid pattern. Each square encompasses a reaction spot of the cellulose membrane region in which a distinct polypeptide sequence has been synthesized in linear form covalently bound to the membrane surface. The relationship between the physical positions in the membrane and the sequence of the polyamino acid is listed in Table 20. These combined polyamino acid sequences represent the encoded sequence of the spike protein of SARS-CoV-2 (S: aa1-1273, A6-K18), ORF3a (ORF3: aa 1-275, K21-N1), membrane glycoprotein (M: aa 1-61, N4-O22); ORF6 (ORF6: aa 1-61, P1-P11); ORF7 (ORF7: aa1-121, P14-Q12), ORF8 (ORF8: aa 1-121, Q15-R13), Nucleocapsid (N: aa1-419, R16-V1), Protein der Energie (E: aa 1-75.), Regs V4-V16), ORF10 (ORF10: aa 1-38, V19-V24). Each polyamino acid has a length of 15 amino acids and an adjacent, continuous overlap of 10 amino acids.

FIG. 15—Reactivity of sera from patients with COVID, revealed with alkaline phosphatase-labeled anti-human IgM antibodies, to SARS-CoV-2 peptides synthesized on cellulose membrane (FIG. 12). 15A, surface glycoprotein; 15B, ORF 3a; 15C, membrane glycoprotein; 15D, ORF 6; 15E, ORF 7; 15F, ORF 8; 15G, nucleoprotein; 15H, protein E; 15I, ORF 10.

FIG. 16—Reactivity of sera from patients with COVID, revealed with alkaline phosphatase-labeled anti-human IgG antibodies, to SARS-CoV-2 peptides synthesized on cellulose membrane (FIG. 13). 16A ORF 3a; 16B: membrane glycoprotein; 16C: ORF 6; 16D: ORF7; 16E: ORF 8; 16F: nucleoprotein; 16G: E protein; 16H: ORF 10.

FIG. 17—Reactivity of sera from patients with COVID, revealed with alkaline phosphatase-labeled anti-human IgG antibodies, to SARS-CoV-2 peptides synthesized on cellulose membrane (FIG. 14). 17A, surface glycoprotein; 17B, ORF 3a; 17C, membrane glycoprotein; 17D, ORF 6; 17E, ORF 7; 17F ORF 8; 17G, nucleoprotein; 17H, protein E; 17I, ORF 10.

FIG. 18—ELISA of serum from hospitalized patients (n=36) (group 3) with branched synthetic peptides (SARS-X1-SARS-X8) revealed with anti-IgM secondary antibodies.

FIG. 19—ELISA of serum from hospitalized patients (n=36) with branched synthetic peptides (SARS-X1-SARS-X8) revealed with anti-IgG secondary antibodies.

FIG. 20—ELISA of serum from hospitalized patients (n=36) with branched synthetic peptides (SARS-X4-SARS-X8) revealed with anti-IgA secondary antibodies.

FIG. 21—ELISA of serum from four patient groups (group 1: asymptomatic, 2: suspected; 3: hospitalized, and 4 immunoprotected) with the SARS-X3 branched synthetic peptide, revealed with anti-IgM secondary antibodies.

FIG. 22—ELISA of serum from four groups of patients (group 1: asymptomatic, 2: suspected; 3: hospitalized, and 4 immunoprotected) with the SARS-X8 branched synthetic peptide, revealed with anti-IgG secondary antibodies.

FIG. 23—ELISA of serum from four groups of patients (group 1: asymptomatic, 2: suspected; 3: hospitalized, and 4 immunoprotected) with the synthetic peptide SARS-X7, revealed with anti-IgA secondary antibodies.

FIG. 24—Polyacrylamide gel (SDS-PAGE) subjected to electrophoresis demonstrating the production of Ag-Covid19, Ag-COVID19 proteins with a tail of six histidines Tx-SARS-IgM, Tx-SARS2-IgG, Tx-SARS2-G/M, Tx-SARS2-IgA, Tx-SARS2-Universal and Tx-SARS2-G5. The vertical columns 1 to 10, indicate: 1) molecular weight; 2) total bacterial extract without induction of recombinant protein; 3) total bacterial extract after induction of Ag-COVID19 production; 4) total bacterial extract after induction of Ag-COVID19 protein production with a six histidine tail; 5) total bacterial extract after induction of Tx-SARS2-IgM protein production; 6) total bacterial extract after induction of Tx-SARS2-IgG protein production; 7) total bacterial extract after induction of Tx-SARS2-G/M protein production; 8) total bacterial extract after induction of Tx-SARS2-IgA protein production; 9) total bacterial extract after induction of Tx-SARS2-Universal protein production and 10) total bacterial extract after induction of Tx-SARS2-production The letters stand for the molecular weight standard of A) 250 kDa; B) 130 kDa; C) 100 kDa; D) 70 kDa; E) 55 kDa; F) 35 kDa and G) 25 kDa.

FIG. 25—Polyacrylamide gel (SDS-PAGE) subjected to electrophoresis demonstrating the purification of Ag-COVID19 protein by affinity chromatography. (Total) Profile of a total bacterial extract after induction of production; (FT) Profile of proteins that did not bind on a nickel column; (200) Elution profile of Ag-COVID19 protein after addition of 200 mM Imidizole; (75) Elution profile of Ag-COVID19 protein after addition of 75 mM Imidizole and (500) Elution profile of Ag-COVID19 protein after addition of 500 mM Imidizole.

FIG. 26—Polyacrylamide gel (SDS-PAGE) submitted to electrophoresis demonstrating the purification of Tx-SARS2-G5 protein by affinity chromatography. (Total) Profile of a total bacterial extract after induction of production; (FT) Profile of proteins that do not bind on a nickel column; (200) Elution profile of Tx-SARS2-G5 protein after addition of 200 mM Imidizole; (75) Elution profile of Tx-SARS2-G5 protein after addition of 75 mM Imidizole and (500) Elution profile of Tx-SARS2-G5 protein after addition of 500 mM Imidizole.

FIG. 27—ELISA of serum from seven groups of patients infected with malaria, dengue fever or SARS-CoV-2 and still admitted (hospitalized), recovered, suspected or asymptomatic. As a control, a set of sera from healthy people, collected prior to the pandemic, was used. Ag-COVID19 protein was used in the ELISA and the binding antibodies were revealed by secondary human anti-IgG antibodies.

FIG. 28—ELISA assay from serum of six groups of patients, with syphilis, malaria, dengue or SARS-CoV2, hospitalized or suspected. As a control, a set of sera from healthy people, collected prior to the pandemic, was used. Tx-SARS2-G5 protein was used in the ELISA and the binding antibodies were revealed by human anti-IgG secondary antibodies.

FIG. 29—Antibody titration by ELISA against Ag-COVID19 in mice two or four weeks after their immunization with Ag-COVID19 protein.

FIG. 30—Antibody purification using Ag-COVID19 protein.

### DETAILED DESCRIPTION OF THE INVENTION

While the present invention may be susceptible to different embodiments, preferred embodiments are shown in the drawings and in the following detailed discussion with the understanding that the present description should be considered an exemplification of the principles of the invention and is not intended to limit the present invention to what has been illustrated and described herein.

Throughout this document some abbreviations will be used. Below is a list of abbreviations:

Regarding Nitrogenous Bases:

C=cytosine; A=adenosine; T=thymidine; G=guanosine

Regarding Amino Acids:

I=isoleucine; L=leucine; V=valine; F=phenylalanine; M=methionine; C=cysteine; A=alanine; G=glycine; P=proline; T=threonine; S=serine; Y=tyrosine; W=tryptophan; Q=glutamine; N=asparagine; H=histidine; E=glutamic acid; D=aspartic acid; K=lysine; R=arginine.

### Protein Receptacle

The present invention is directed to the production and use of a protein receptacle, based on the sequence of a green fluorescent protein, herein called GFP, in a variety of methods and compositions that exploit the ability of said protein receptacle to concurrently present several different or identical exogenous polyamino acid sequences at more than four different protein sites, and furthermore, to exhibit adequate fluorescence intensities, to be efficiently expressed in cellular protein production systems, and to be useful as a reagent for research, diagnosis, or in vaccine compositions.

In a first embodiment, the invention relates to a stable protein structure that supports, at different sites, the insertion of four or more exogenous polyamino acid sequences simultaneously. In another embodiment, the protein receptacle comprises the amino acid sequence shown in SEQ ID NO: 1. In another embodiment, the protein receptacle comprises the amino acid sequence shown in SEQ ID NO: 3. In another embodiment, the protein receptacle comprises the amino acid sequence shown in SEQ ID NO: 77. In another embodiment, the protein receptacle presents insertion sites for the exogenous polyamino acid sequences in protein loops facing the external environment. In another embodiment, the insertion of the exogenous polyamino acid sequences simultaneously does not interfere with the production conditions of the receptor protein. In another embodiment, the protein receptacle contains exogenous polyamino acid sequences simultaneously for use in vaccine compositions, in diagnostics, or in the development of laboratory reagents. In another embodiment, the exogenous polyamino acid sequences did not lose their immunogenic characteristics when simultaneously inserted into the protein loops of the receptacle protein. In another embodiment, the protein receptacle comprises the exogenous polyamino acid sequences SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15 and SEQ ID NO: 16, simultaneously. In another embodiment, the protein receptacle comprises the amino acid sequence shown in SEQ ID NO: 18. In another embodiment, the protein receptacle comprises the exogenous polyamino acid sequences SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28 and SEQ ID NO: 29, simultaneously. In another embodiment, the protein receptacle comprises the amino acid sequence shown in SEQ ID NO: 20. In another embodiment, the protein receptacle comprises several copies of the exogenous polyamino acid sequence simultaneously, SEQ ID NO: 30. In another embodiment, the protein receptacle comprises the amino acid sequence shown in SEQ ID NO: 31. In another embodiment, the protein receptacle comprises the exogenous polyamino acid sequences SEQ ID NO: 35, SEQ ID NO: 36, SEQ ID NO: 37, SEQ ID NO: 38, SEQ ID NO: 39 and SEQ ID NO: 40, simultaneously. In another embodiment, the protein receptacle comprises the amino acid sequence shown in SEQ ID NO: 33. In another embodiment, the protein receptacle comprises the exogenous polyamino acid sequences SEQ ID NO: 41, SEQ ID NO: 42, SEQ ID NO: 43 and SEQ ID NO: 44, simultaneously. In another embodiment, the protein receptacle comprises the amino acid sequence shown in SEQ ID NO: 45. In another embodiment, the protein receptacle comprises the exogenous polyamino acid sequences SEQ ID NO: 47, SEQ ID NO: 48, SEQ ID NO: 49 and SEQ ID NO: 50, simultaneously. In another embodiment, the protein receptacle comprises the amino acid sequence shown in SEQ ID NO: 51. In another embodiment, the protein receptacle comprises the exogenous polyamino acid sequences SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 95 and SEQ ID NO: 96, simultaneously. In another embodiment, the protein receptacle comprises the amino acid sequence shown in SEQ ID NO: 64. In another embodiment, the protein receptacle comprises the exogenous polyamino acid sequences SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74 and SEQ ID NO: 97, simultaneously. In another embodiment, the protein receptacle comprises the amino acid sequence shown in SEQ ID NO: 75. In another embodiment, the protein receptacle comprises the exogenous polyamino acid sequences, simultaneously, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87 and SEQ ID NO: 98. In another embodiment, the protein receptacle comprises the amino acid sequence shown in SEQ ID NO: 88. In another embodiment, the protein receptacle comprises the exogenous polyamino acid sequences SEQ ID NO: 7, SEQ ID NO: 9, SEQ ID NO: 10, SEQ ID NO: 12, SEQ ID NO: 14, SEQ ID NO: 15, SEQ ID NO: 16, SEQ ID NO: 92, SEQ ID NO: 93, SEQ ID NO: 94 and SEQ ID NO: 99, simultaneously. In another embodiment, the protein receptacle comprises the amino acid sequence shown in SEQ ID NO: 90. In another embodiment, the protein receptacle comprises the exogenous polyamino acid sequences as defined in SEQ ID NO: 100, 124, 125, and 126, simultaneously; or SEQ ID NO:101, 127, 128, 129, 130, 131, 132, 133, 134, and 135, simultaneously; or SEQ ID NO: 136, 137, 138, 139, 140, 141, 142, simultaneously; or SEQ ID NO: 129, 133, 135, 137, 140, 141, 142, 143, 144, 146, 147, and 148, simultaneously; or SEQ ID NO: 103, 149, 150, 151, 152, 153, 154, 155, simultaneously; or SEQ ID NO: 104, 156, 157, 158, 159, 160, 161, 162, and 163, simultaneously; or SEQ ID NO: 136, 139, 140, 141, 142, 143, 144, 146 and 147, simultaneously. In another embodiment, the protein receptacle comprises any of the amino acid sequences shown in SEQ ID NO: 334-341.

Another embodiment of the present invention relates to the efficient expression of said protein receptacle, based on the sequence of a GFP, carrying one or two, or more than two, three or four, or more than ten exogenous polyamino acid sequences, concomitantly, at ten different sites on the receptacle protein, in a cellular system. Specifically, the present invention relates to the efficient expression of said protein receptacle presenting exogenous polyamino acid sequences at up to ten different protein sites simultaneously. More specifically, the present invention relates to the efficient expression of said receptacle carrying said exogenous polyamino acid sequences, inserted into different protein sites, without, however, losing its inherent characteristics, such as, autofluorescence.

The invention is also directed to the production and use of the protein receptacles, “Platform”, “Rx” and “Tx”, and their amino acid sequences (described in SEQ ID NO: 1, SEQ ID NO: 3 and SEQ ID NO: 77, respectively), of their nucleotide sequences (described in SEQ ID NO: 2, SEQ ID NO: 4 and SEQ ID NO: 78, respectively) and their amino acid sequences including the exogenous polyamino acid sequences of choice (described in SEQ ID NO: 18, SEQ ID NO: 20, SEQ ID NO: 31, SEQ ID NO: 33, SEQ ID NO: 45, SEQ ID NO: 51, SEQ ID NO: 64, SEQ ID NO: 75, SEQ ID NO: 88 and SEQ ID NO: 90). The receptacle protein can also undergo modifications necessary for the development of its uses, through the insertion of accessory elements. Sequences can be added to the receptacle protein that aid in the purification process, such as, but not limited to, polyhistidine tail, chitin-binding protein, maltose-binding protein, calmodulin-binding protein, strep-tag, and GST. Sequences for stabilization such as thiorodixin can still be incorporated into the receptor protein. Sequences that aid in any antibody detection process, such as V5, Myc, HA, Spot, FLAG sequences, can also be added to the receptacle protein.

Further, for purposes of this invention, sequences with at least about 85%, more preferably at least about 90%, 95%, 96%, 97%, 98% or 99% identity with the protein and polyamino acid receptors described herein are included, as measured by well-known sequence identity assessment algorithms such as FASTA, BLAST or Gap.

Sequences acting as target cleavage sites (catalytic site) for proteases may still be added to the protein receptacle, allowing the main protein to be separated from the above accessory elements, included strictly for the purpose of optimizing the production or purification of the protein, but not contributing to the suggested end use. These sequences, containing sites that serve as targets for proteases, can be inserted anywhere and include, but are not limited to, thrombin, factor Xa, enteropeptidase, PreScission, TEV (Kosobokova et al., Biochemistry, 8: 187-200, 2015). Yet another accessory sequence marking proteases can be AviTag, which allows specific biotinylation at a single point during or after protein expression. In this sense, it is possible to create tagged proteins by combining different elements (Wood, Current Opinion in Structural Biology, 26: 54-61, 2014).

The isolated receptacle protein can be further modified in vitro for different uses.

### Polynucleotide

In a first embodiment, the invention relates to a polynucleotide comprising any one of SEQ ID NO: 2, 4, 78, 17, 19, 32, 34, 46, 52, 63, 76, 89, 91, 326-333 and their degenerate sequences, capable of generating, respectively, the polypeptides defined by SEQ ID NO: 1, 3, 77, 18, 20, 31, 33, 45, 51, 64, 75, 88, 90, 334-341.

This invention also provides an isolated receptacle protein, produced by any expression system, from a DNA molecule, comprising a regulatory element containing the nucleotide sequence encoding the receptacle protein of choice.

The DNA sequence encoding for the receptacle protein differs from the DNA sequence of forms of GFP that occur in nature, in terms of the identity or location of one or more amino acid residues, either by deletion, addition, or substitution of amino acids. But, they still preserve some or all of the characteristics inherent to forms that occur in nature, such as, but not limited to, fluorescence production, characteristic three-dimensional shape, ability to be expressed in different systems, and ability to receive exogenous peptides.

The DNA sequence encoding for the receptacle protein of the present invention includes: the incorporation of preferred codons for expression by certain expression systems; the insertion of cleavage sites for restriction enzymes; the insertion of optimized sequences for facilitating expression vector construction; the insertion of facilitator sequences to house the polyamino acid sequences of choice to be introduced into the receptacle protein. All these strategies are already well known in the state of the art.

Additionally, the invention further provides added genetic elements of the nucleotide sequences encoding the receptacle proteins, such as in the sequences described in SEQ ID NO: 2, SEQ ID NO: 4 and SEQ ID NO: 78. Yet additionally, it provides such elements containing nucleotide sequences encoding the added receptacle proteins of the DNA coding for the exogenous polyamino acid sequences of choice. Such genetic elements containing the sequences described in SEQ ID NO: 17, SEQ ID NO: 19, SEQ ID NO: 32, SEQ ID NO: 34, SEQ ID NO: 46, SEQ ID NO: 52, SEQ ID NO: 63, SEQ ID NO: 76, SEQ ID NO: 89 and SEQ ID NO: 91.

Regulatory elements required for receptacle protein expression include promoter sequences for binding to RNA polymerase and translation start sequences for binding to the ribosome. For example, a bacterial expression vector must include a primer codon, a promoter suitable for the cellular system, and for translation initiation a Shine-Dalgarno sequence. Similarly, a eukaryotic expression vector includes a promoter, a start codon, a polyadenylation signal downstream process, and a termination codon. Such vectors can be obtained commercially, or built from already known state-of-the-art sequences.

### Vector

In a first embodiment, the invention relates to a vector comprising the polynucleotide as previously defined.

Transition from one plasmid to another vector can be achieved by altering the nucleotide sequence by adding or deleting restriction sites without modifying the amino acid sequence, which can be accomplished by nucleic acid amplification techniques.

### Expression Cassette

In a first embodiment, the invention relates to an expression cassette comprising the polynucleotide as previously defined.

Optimized expression in other systems can be achieved by altering the nucleotide sequence to add or delete restriction sites and also to optimize codons for alignment to the preferred codons of the new expression system, without, however, changing the final amino acid sequence.

### Cell

In a first embodiment, the invention relates to a cell comprising the vector or expression cassette as previously defined.

The invention further provides cells containing nucleotide sequences encoding the receptacle protein, or the receptacle protein added from the DNA encoding for the exogenous polyamino acid sequences of choice, to act as expression systems for the receptacle protein. The cells can be bacterial, fungal, yeast, insect, plant or even animal cells. The DNA sequences encoding the receptacle proteins added from the coding DNA for the exogenous polyamino acid sequences of choice can also be inserted into viruses that can be used for expression and production of the receptacle protein as delivery systems, such as baculovirus, adenovirus, adenovirus-associated viruses, alphavirus, herpes virus, pox virus, retrovirus, lentivirus, but not limited to these.

There is a wide variety of methods for introducing exogenous genetic material into cells, all already well known in the state of the art. For example, exogenous DNA material can be introduced into a cell by calcium phosphate precipitation technology. Other technologies, such as technologies using electroporation, lipofection, microinjection, retroviral vectors, and other viral vector systems, such as adenovirus-associated viral systems may be used for development of this invention.

This invention provides a living organism comprising at least cells containing a DNA molecule containing a regulatory element for expression of the sequence encoding the receptacle protein. The invention is applicable for production of receptacle proteins in vertebrate animals, non-vertebrate animals, plants and microorganisms.

Expression of the receptacle proteins can be performed in, but not limited to, Escherichia coli cells, Bacillus subtilis, Saccharomyces cerevisiae, Pichia pastoris, Pichia methanolica, Candida boidinii, Pichia angusta, mammalian cells such as CHO cells, HEK293 cells or insect cells such as Sf9 cells. All known state-of-the-art prokaryotic or eukaryotic protein expression systems are capable of producing the receptacle proteins.

In one development of the present invention, a virus or bacteriophage, carrying the coding sequence for the receptacle protein, can infect a particular type of bacterial or eukaryotic cell and provide expression of the receptacle protein in that cellular system. Infection can be easily observed by detecting the expression of the receptacle protein. Similarly, a eukaryotic plant or animal cell virus carrying the sequence encoding the receptacle protein can infect a specific cell type and lead to expression of the receptacle protein in the eukaryotic cell system.

### Method for Producing the Protein Receptacle

In a first embodiment, the invention relates to a method for producing the protein receptacle, comprising introducing into competent cells of interest the polynucleotide as previously defined; performing culture of the competent cells and performing isolation of the protein receptacle containing the exogenous polyamino acids of choice. In another embodiment, the protein receptacle is not interfered with by the insertion of various exogenous polyamino acid sequences.

Receptor proteins can also be produced by multiple synthetic biology systems. The generation of fully synthetic genes is linked to basically three linkage-based synthesis systems, widely described in the state of the art.

This invention provides methods for producing the receptacle protein using a protein expression system comprising: introducing into competent cells of interest the DNA sequence encoding for the receptacle protein plus the DNA encoding for the exogenous polyamino acid sequences of choice, culturing these cells under conditions favorable for producing the receptacle protein containing the polyamino acid sequences of choice, and isolating the receptacle protein containing the exogenous polyamino acids of choice.

The invention further provides techniques for producing the receptacle proteins containing the exogenous polyamino acids of choice. This invention presents an efficient method for expressing receptacle proteins containing the exogenous polyamino acids of choice which promotes the production of the protein of interest in large quantities. Methods for the production of the receptor proteins can be performed in different cellular systems, such as yeast, plants, plant cells, insect cells, mammalian cells, and transgenic animals Each system can be used by incorporating a codon-optimized nucleic acid sequence, which can generate the desired amino acid sequence, into a plasmid appropriate for the particular cell system. This plasmid may contain elements, which confer a number of attributes on the expression system, which include, but are not limited to, sequences that promote retention and replication, selectable markers, promoter sequences for transcription, stabilizing sequences of the transcribed RNA, ribosome binding site.

Methods for isolating expressed proteins are well known in the state of the art, and in this regard, receptacle proteins can be easily isolated by any technique. The presence of the polyhistidine tail allows purification of the recombinant proteins after their expression in the bacterial system (Hochuli et al Bio/Technology 6: 1321-25; Bornhorst and Falke, Methods Enzymology 326:245-54).

The present invention further contemplates the choice and selection of exogenous polyamino acids. Receptor proteins can house different polyamino acid sequences from different sources, ranging from vertebrate animals including mammals, invertebrates, plants, microorganisms or viruses in order to promote their expression, presentation or use in different media. Different methods of polyamino acid sequence selection can be used, such as specific selection by binding affinity to antibodies or other binding proteins, by epitope mapping, or other techniques known in the state of the art.

Polyamino acid sequences are sequences of five to 30 amino acids that sensitively or specifically represent an organism, for any purpose described in this patent application. Polyamino acid sequences may represent, but not limited to, these examples: (i) linear B-cell epitopes; (ii) T-cell epitopes; (iii) neutralizing epitopes; (iv) protein regions specific to pathogenic or non-pathogenic sources; (v) regions proximal to active sites of enzymes that are not normally targets of an immune response. These epitope regions can be identified by a wide variety of methods that include, but are not limited to: spot synthesis analysis, random peptide libraries, phage display, software analysis, use of X-ray crystallography data, epitope databases, or other state-of-the-art methods.

Insertion of exogenous polyamino acid sequences into the receptacle proteins at previously identified sites surprisingly did not disrupt or interfere with the inherent characteristics of the protein. Eight introduction sites for exogenous polyamino acid sequences have been identified in the receptacle proteins (Kiss et al. Nucleic Acids Res 34: e132, 2006; Pavoor et al. Proc Natl Acad Sci USA 106: 11895-900, 2009; Abedi et al, Nucleic Acids Res 26: 623-30, 1998; Zhong et al. Biomol Eng 21: 67-72, 2004). These introduction sites can house one, two, or more different exogenous polyamino acid sequences in tandem at the same insertion site, greatly amplifying the expression of different polyamino acid sequences.

### Method of Pathogen Identification or In Vitro Disease Diagnosis

In a first embodiment, the invention relates to a method for pathogen identification or in vitro disease diagnosis characterized in that it uses the receptacle protein as previously defined. In another embodiment, the method is for diagnosing Chagas disease, rabies, pertussis, yellow fever, Oropouche virus infections, Mayaro, IgE hypersensitivity, D. pteronyssinus allergy, or COVID-19.

### Use of the Protein Receptacle

In a first embodiment, the invention relates to the use of said protein receptacle as a laboratory reagent. In a further embodiment, the invention relates to the use of said protein receptacle for the production of a vaccine composition for immunization against Chagas disease, rabies, pertussis, yellow fever, Oropouche virus infections, Mayaro virus infections, IgE hypersensitivity, D. pteronyssinus allergy or COVID-19.

Also, the present invention relates to a system for concomitant expression of multiple polyamino acid sequences using a single protein receptacle, based on GFP, useful for different uses, such as a reagent for research, for diagnosis or for vaccine compositions. Specifically, said expression system can act as a useful research reagent to purify antibodies by binding on epitopes. Additionally, said expression system can also act for use in immunological and/or molecular techniques for diagnosing chronic and infectious diseases. Also, said expression system may be useful for use in vaccine compositions containing multiple antigens for immunization of animals and humans.

Also an embodiment of the present invention is the method for concomitant production of multiple polyamino acid sequences using a single protein receptacle, based on GFP, useful for different uses, such as a reagent for research, for diagnostics or for vaccine compositions.

The invention further presents several uses for the receptacle protein. These uses include, but are not limited to, the use of the protein receptacle (i) as a reporter molecule in cellular screening assays, including intracellular assays; (ii) as a protein for presentation of random or selected peptide libraries; (iii) as an antigen-presenting protein as a reagent for development of in vitro immunological diagnostic tests, in general, and for infectious, parasitic or other immunological diseases; (iv) as an antigen-presenting protein useful for selection, capture, screening or purification of binding substances, such as, antibodies; (v) as an antigen-presenting protein for vaccine composition; (vi) as a protein containing antibody sequences for binding to antigens; (vii) as an antigen-presenting protein with passive immunization activity.

Receptor proteins can be useful as a vaccine composition by, unusually, having: (a) a large number, simultaneously, of immune response-inducing polyamino acid sequences, and (b) a nonimmune response-inducing protein core.

### Diagnostic Kit

In a first embodiment, the invention relates to a diagnostic kit comprising the protein receptacle as previously defined.

Finally, the present invention is described in detail through the examples given below. It is necessary to stress that the invention is not limited to these examples, but that it also includes variations and modifications within the limits within which it can be developed. It is also worth mentioning that the access to all biological sequences of the Brazilian genetic heritage are registered in SISGEN, under the registration number AC53976.

### EXAMPLES

### Example 1—Receptacle Protein Construction

The amino acid sequences between different examples of protein fluorescent green, eGFP (GenBank: L29345.1; UniProtKB—P42212), Cycle-3 (GenBank: CAH64883.1), SuperFolder (GenBank: AOH95453.1), Split (Cabantous et al. Science Reports 3: 2854, 2013), Superfast (Fisher & DeLisa. PLoS One 3: e2351, 2008) were used to construct new proteins for the present invention application. Sequence alignments and comparisons were performed by Intaglio software (Purgatory Design, V3.9.4). From these data, several changes were made so that the receptacle proteins could achieve the required characteristics.

Alterations were made to create restriction enzyme action sites. The insertion of these sites was designed so that there would be no change in the physicochemical characteristics of the GFP protein and thus not affect the properties or qualities described in this patent application. Additionally, the insertion of these restriction sites adds further properties to the receptor proteins by allowing potential uses in genetic engineering methods and processes that would allow genetic manipulation of these proteins for incorporation of various peptides into different regions of the protein.

The nucleotide sequence of the GFP protein was manipulated to introduce or replace nucleotides in order to create new restriction enzyme sites. Thus, two new receptacle proteins were produced, the “Platform” protein and the “Rx” protein.

The changes in nucleotide sequences based on the eGFP protein, gave rise to the following amino acid changes in the receptacle proteins:

Platform″ Protein
Position 16, amino acid I;
Position 28, amino acid F;
Position 30, amino acid R;
Position 39, amino acid I;
Position 43, amino acid S;
Position 72, amino acid S;
Position 99, amino acid Y;
Position 105, amino acid T;
Position 111, amino acid E;
Position 124, amino acid V;
Position 128, amino acid I;
Position 145, amino acid F;
Position 153, amino acid T;
Position 163, amino acid A;
Position 166, amino acid T;
Position 167, amino acid V;
Position 171, amino acid V;
Position 205, amino acid T;
Position 206, amino acid I;
Position 208, amino acid L.

Rx″ Protein
Position 16, amino acid V;
Position 28, amino acid S;
Position 30, amino acid R;
Position 39, amino acid I;
Position 43, amino acid T;
Position 72, amino acid A;
Position 99, amino acid S;
Position 105, amino acid K;
Position 111, amino acid V;
Position 124, amino acid V;
Position 128, amino acid T;
Position 145, amino acid F;
Position 153, amino acid T;
Position 163, amino acid A;
Position 166, amino acid T;
Position 167, amino acid V;
Position 171, amino acid V;
Position 205, amino acid T;
Position 206, amino acid V;
Position 208, amino acid S.

Alternative amino acid substitutions can still be selected, for all the receptacle proteins, at positions:
Position 39, amino acid N;
Position 72, amino acid S;
Position 99, amino acid S;
Position 105, amino acid Y or K;
Position 206, amino acid I;
Position 208, amino acid L.

The presence of some mutations can influence biochemical characteristics of the protein. The S30R mutation positively influences the protein's coiling characteristics; the Y145F and I171V mutations prevent translation of undesirable intermediates; the A206V or I mutations reduce the possibility of aggregation of nascent proteins.

Other changes were made to the receptacle proteins “Platform” and “Rx” from the inclusion of new nucleotide codons to create new restriction sites, which can be seen in Table 1 (below):

TABLE 1

| Amino acid | | | Restriction |
|---|---|---|---|
| sequence | Substituted | Nucleotides | enzyme to be |
| variation | nucleotides | introduced | used |
| D102_D103insV | — | GTC | AatII |
| G116_D117insT | — | ACC | KpnI |
| L137_G138insK | — | AAG | AfIII |
| D191_P192insG | — | GGT | RsrII |
| E213_K214insL | — | CTC | SacI |

Additionally, the nucleotide sequence of the receptacle proteins “Platform” and “Rx” harbors two additional restriction sites for NdeI and NheI, at the amino 5′ terminus of the protein, from the insertion of the sequence CATATGGTGGCTAGC (SEQ ID NO: 5) and another two restriction sites for EcoRI and XhoI, at the 3′ carboxy terminus, from the insertion of the sequence GAATTCTAATGACTCGAG (SEQ ID NO: 6). In addition, two stop codons and a polyhistidine tail at the amino-terminus have been incorporated into the receptor proteins.

The amino acid sequence of the “Platform” protein can be seen in SEQ ID NO: 1 and its corresponding sequence in nucleotides is described in SEQ ID NO: 2.

The amino acid sequence of the “Rx” protein can be seen in SEQ ID NO: 3 and its corresponding sequence in nucleotides is described in SEQ ID NO: 4.

By creating restriction sites without altering the three-dimensional structure of the original protein, 10 new insertion sites for exogenous polyamino acid sequences were allowed to appear in the protein. Each new insertion site will be referred to here as position 1 to position 10.

The locations, in the nucleotide and amino acid sequences, of positions 1 to 10, of the receptacle proteins “Platform” and “Rx” are shown in Table 2 (below):

TABLE 2

| Position in the | Position in the |
|---|---|
| protein receptacle | amino acid sequence |
| 1 | MVAS |
| 2 | TTGKLPVP |
| 3 | FKDVDG |
| 4 | FEGTDTL |
| 5 | TDFKEDGNILKGHKL |
| 6 | DKQKN |
| 7 | ED |
| 8 | PIGDGPVLLPDN |
| 9 | SKDPNELKRD |
| 10 | DELYKEF |

From alignments of the amino acid sequences of different GFP proteins, a consensus amino acid sequence, designated CGP (Dai et al. Protein Engineering, Design and Selection 20(2): 69-79 2007). This fluorescent protein although exhibiting high stability, was improved by directed evolution to exhibit greater stability relative to CGP (Kiss et al. Protein Engineering, Design & Selection 22(5): 313-23, 2009). However, the enhanced protein, due to the presence of three mutations, was prone to aggregation. Further mutations were also incorporated based on an analysis of its crystal structure resulting in the elimination of aggregation and production of the protein called Thermal Green Protein (TGP) (Close et al. Proteins 83(7): 1225-37, 2015). When used as a protein receptacle, the sequence is called “Tx”. The amino acid sequence of the “Tx” protein can be seen in SEQ ID NO: 77 and its corresponding sequence in nucleotides is described in SEQ ID NO: 78.

The nucleotide and amino acid sequence locations of positions 1 to 13 of the “Tx” receptacle proteins are shown in Table 3 (below). At the amino and carboxy-terminal ends of the receptacle proteins, two polyamino acid sequences can be inserted consecutively at both ends. Thus, insertion sites 1a and 1b and 13a and 13b are characterized in the amino and carboxy-terminal regions, respectively.

TABLE 3

| Position in the | Position in the |
|---|---|
| protein receptacle | amino acid sequence |
| 1 | GAHASVIKPE |
| 2 | NG |
| 3 | YE |
| 4 | GAPLPFS |
| 5 | AFPE |
| 6 | EDQ |
| 7 | GD |
| 8 | NFPPNGPVMQKK |
| 9 | DG |
| 10 | EGGG |
| 11 | KKDVRLPDA |
| 12 | DKDYN |
| 13 | RYSG |

### Example 2—Construction of the PlatCruzi Protein

The “Platform” receptacle protein was genetically engineered to harbor Trypanosoma cruzi epitopes, which herein we call the PlatCruzi platform. The gene corresponding to the PlatCruzi protein, here called the PlatCruzi gene, is described in the SEQ ID NO nucleotide sequence: 17.

Polyamino acid sequences originating from T. cruzi were selected from the available state-of-the-art literature, considering experimental data on specificity and sensitivity for diagnostic tests for Chagas disease (Peralta J M et al. J Clin Microbiol 32: 971-974, 1994; Houghton R L et al. J Infect Dis 179: 1226-1234, 1999; Thomas et al. Clin Exp Immunol 123: 465-471, 2001; Rabello et al, 1999; Gruber & Zingales, Exp Parasitology, 76(1): 1-12, 1993; Lafaille et al, Molecular Biochemistry Parasitology, 35(2): 127-36, 1989). Ten polyamino acid sequences were selected for insertion into the ten insertion sites in the “Platform” protein, here called TcEp1 to TcEp10, as shown in Table 4 (below).

After the selection of the T. cruzi polyamino acid sequences, the synthetic gene corresponding to the PlatCruzi protein was produced by chemical synthesis, by the ligation gene synthesis methodology and inserted into plasmids for experimentation. The amino acid sequence corresponding to the PlatCruzi gene, containing the epitopes TcEp1 to TcEp10, is described in SEQ ID NO: 18.

TABLE 4

| | Position in | Original | | |
|---|---|---|---|---|
| Epitope | the protein | epitope protein | Sequence | SEQ ID no. |
| TcEp1 | 1 | KMP11 | KFAELLEQQKNAQFPGK | SEQ ID no. 7 |
| TcEp2 | 2 | TcE | KAAAAPA | SEQ ID no. 8 |
| TcEp3 | 3 | TcE | KAAIAPA | SEQ ID no. 9 |
| TcEp4 | 4 | PEP-2 | GDKPSPFGQAAAADK | SEQ ID no. 10 |
| TcEp5 | 5 | CRA | KQKAAEATK | SEQ ID no. 11 |
| TcEp6 | 6 | TcD-1 | AEPKPAEPKS | SEQ ID no. 12 |
| TcEp7 | 7 | TcD-2 | AEPKSAEPKP | SEQ ID no. 13 |
| TcEp8 | 8 | TcLo 1.2 | GTSEEGSRGGSSMPS | SEQ ID no. 14 |
| TcEp9 | 9 | B13 | SPFGQAAAGDK | SEQ ID no. 15 |
| TcEp10 | 10 | CRA | KQRAAEATK | SEQ ID no. 16 |

### Example 3—Development of the PlatCruzi Protein

The synthetic gene was introduced into pET28a plasmids using the restriction sites for the enzymes NdeI and XhoI by state-of-the-art molecular biology methods. In order to identify whether the synthetic gene matched the sequence designed for PlatCruzi, a DH5α strain of Escherichia coli was transformed and the plasmid material analyzed by restriction enzyme digestion techniques and then sequenced.

The sequencing method used was the enzymatic, dideoxy or chain termination method, which is based on the enzymatic synthesis of a complementary strand, the growth of which is stopped by the addition of a dideoxynucleotide (Sanger et al., Proceeding National Academy of Science, 74(12): 5463-5467, 1977). This methodology consists of the following steps: Sequencing reaction (DNA replication in 25 cycles in the thermocycler), DNA precipitation by isopropanol/ethanol, denaturation of the double strand (95° C. for 2 min) and reading of the nucleotide sequence was performed in the ABI 3730XL automated sequencer (ThermoFischer SCIENTIFIC) (Otto et al., Genetics and Molecular Research 7: 861-871, 2008). The analyses of the obtained sequences were done with the help of the 4Peaks program (Nucleobytes; Mac OS X, 2004). The primers from the pET-28a vector (T7 Promoter and T7 Terminator) were used in the reaction.

The analyzed plasmid clone harboring the correct sequence for PlatCruzi was transferred to E. coli, strain BL21, in order to produce the PlatCruzi protein. The BL21 strain can express T7 RNA polymerase when induced by Isopropyl β-D-1-thiogalactopyranoside (IPTG). The strain was grown overnight in LB medium and then reseeded in the same medium, with kanamycin (30 μg/ml) added, on a shaker at 200 rpm until it reached an optical turbidity density of 0.6-0.8 (600 nm). Then, IPTG (q.s.p. 1 mM) is added to the culture and the same culture conditions are maintained for another 3 h.

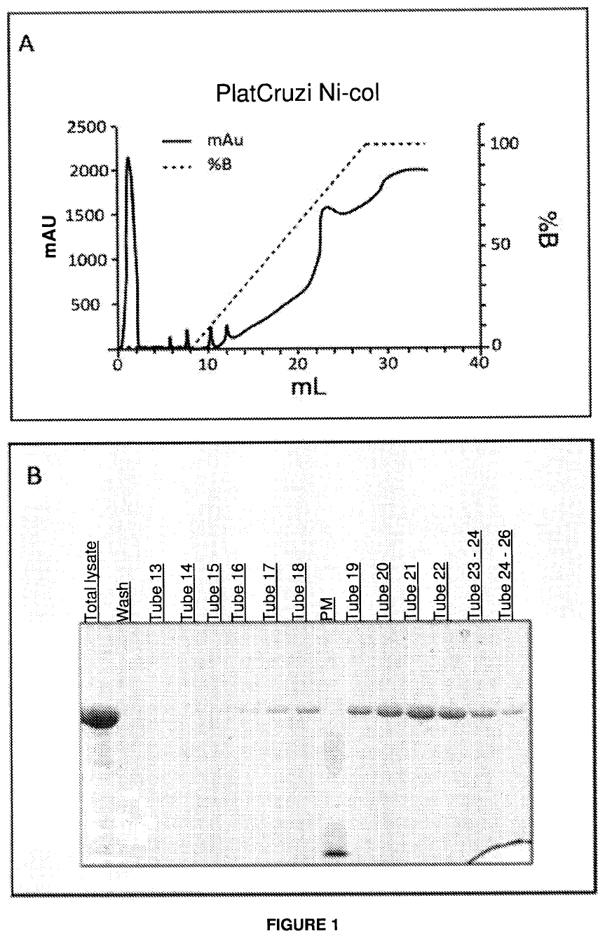

The culture was subjected to centrifugation and the pellet resuspended in urea buffer (100 mM NaH2PO4, 10 mM Tris-base, 8 M urea, pH 8.0). The solution was subjected to HisTrap™ affinity column chromatography, 1 mL, (GE Healthcare Life Sciences) which allows high-resolution purification of histidine-tagged proteins. The supernatant was applied to a nickel affinity column (HisTrap™, 1 mL, GE Healthcare Life Sciences) at a flow rate of 0.5 mL per minute, previously equilibrated in buffer A (50 mM Tris-HCl, pH 8.0, 100 mM NaCl and 5 mM imidazole). After binding, the resin was washed with 10 mL of buffer A. The protein was eluted in a 100% gradient of buffer B (50 mM Tris-HCl, pH 8.0, 100 mM NaCl, and 500 mM imidazole) at a flow rate of 0.7 mL/min for 45 minutes. The purification of PlatCruzi was followed at 280 nm (black line) and is shown in FIG. 1A. The percentage of imidazole is marked in red. The protein was eluted in a volume of approximately 19 to 25 ml.

Aliquots of the recombinant proteins (1 μg/well) were subjected to SDS-containing polyacrylamide gel electrophoresis (SDS-PAGE) (Laemmli, Nature 227: 680-685, 1970). Concentration gels (stacking gel) and separation gels (running gel) were prepared at an acrylamide concentration of 4% and 11%, respectively (table 5, below). Samples were prepared under denaturing conditions in 62.5 mM Tris-HCl buffer, pH 6.8, 2% SDS, 5% β-mercaptoethanol, 10% glycerol and boiled at 95° C. for 5 min (Hames B D, Gel electrophoresis of proteins: a practical approach. 3. ed. Oxford. 1998). After electrophoresis, the proteins were detected by staining with comassie blue R250 (Bio-Rad, USA). The Kaleidoscope™ Prestained Standards marker was used as a molecular weight reference (Bio-Rad, USA). FIG. 1B, shows the purification of PlatCruzi protein by affinity chromatography using a nickel-agarose column using a Äktapurifier liquid chromatography system.

TABLE 5
Volume and concentration of reagents used to prepare the 4%
sample concentration gel and for the 11% separation gel.

| 4% concentration gel | | Separation Gel 11 | |
|---|---|---|---|
| | Volume | | Volume |
| Reagents | (mL) | Reagents | (mL) |
| H2O | 1.63 | H2O | 1.44 |
| 0.5M Tris-HCl, pH 6.8 | 0.833 | 1.5M Tris-HCl, pH 8.8 | 1.4 |
| 30% Acrylamide/0.8% | 0.5 | 30% Acrylamide/0.8% | 2 |
| Bisacrylamide | | Bisacrylamide | |
| 10% SDS (sodium duo- | 0.0333 | 10% SDS (sodium duo- | 0.055 |
| decyl sulfate) | | decyl sulfate) | |
| 10% Ammonium | 0.013 | 10% Ammonium | 0.015 |
| Persulfate | | Persulfate | |
| 80% Glycerol | 0.333 | 80% Glycerol | 0.55 |
| TEMED | 0.0067 | TEMED | 0.0075 |

### Example 4—Development of an ELISA from PlatCruzi

The performance of PlatCruzi protein was evaluated against a panel of reference biological samples and individuals affected by T. cruzi infections. PlatCruzi protein, in carbonate/bicarbonate buffer solution (50 mM, pH 9.6), was added to a 96-well ELISA plate in the amounts of 0.1, 0.25, 0.5 and 1.0 μg/orifice at 4° C. for 12-18 h. The wells were washed with saline-phosphate buffer (PBS) solution added Tween 20 (PBS-T, 10 mM sodium phosphate—Na3PO4, 150 mM sodium chloride—NaCl and 0.05% Tween-20, pH 7.4) and then incubated with 1×PBS buffer containing 5% (weight/volume) dehydrated skim milk for 2 h at 37° C.

The wells were then washed 3 times with PBS-T buffer and incubated with the reference biological samples TCI (IS 09/188) or TCII (IS 09/186) (World Health Organization) diluted 2, 4, 8, 16, 32 and 64 times for 1 h at 37° C. After the incubation period, the wells were washed three times with PBS-T and then incubated with alkaline phosphatase-labeled human IgG antibody at a dilution of 1:5000 for 1 h at 37° C. The wells were washed again three times with PBS-T buffer and the substrate para-nitrophenylphosphate (PNPP, 1 mg/mL, ThermoFischer SCIENTIFIC) was added. After 30 minutes and under shelter from light, the absorbance was measured in an ELISA plate reader at 405 nm.

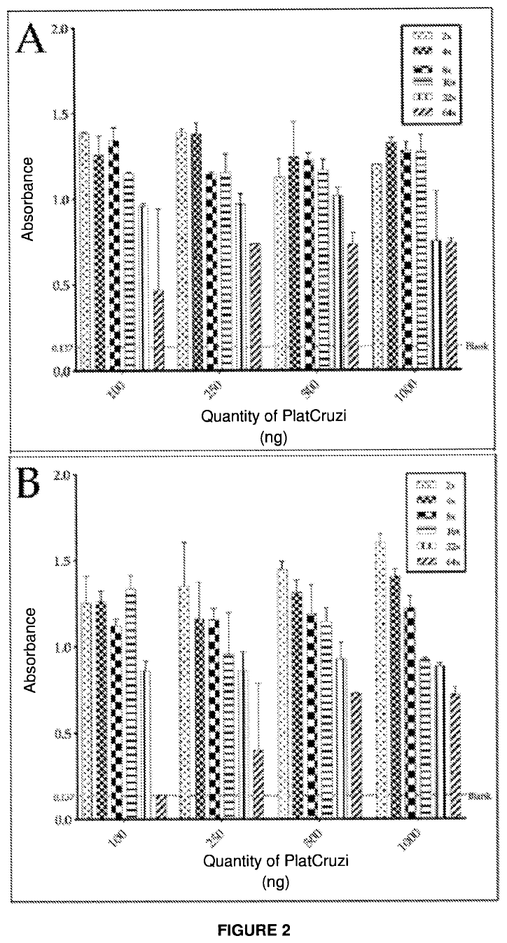

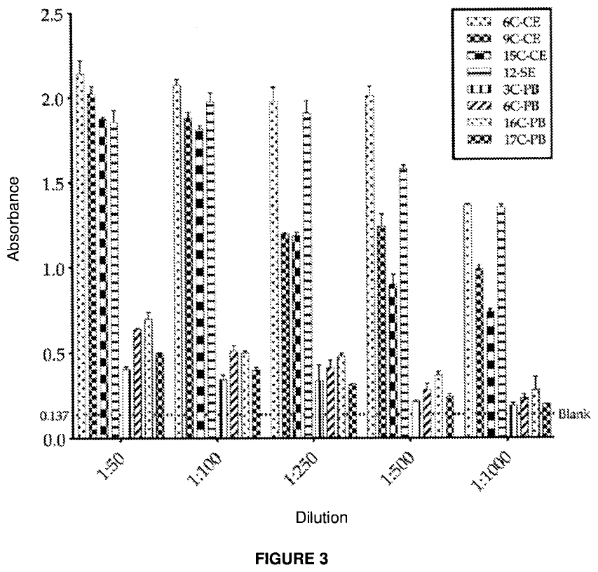

The results showed satisfactory responses in all dilutions of TCI and TCII reference biological samples used regardless of the amount of PlatCruzi used (FIGS. 2A and 2B). The results strongly support the use of PlatCruzi for detection of T. cruzi infections caused by any of the six DTUs (discrete typing units), covering the entire geographic range of circulating T. cruzi strains. The same performance can be observed when using sera from Chagas disease patients, with low and high antibody detection titers (FIG. 3).

Elisa plates containing 500 ng (in 0.3M Urea, pH 8.0) of PlatCruzi were prepared as previously described and, after three washes with PBS-T, were incubated with sera from four patients with high anti-T. cruzi antibody indices (6C-CE, 9C-CE, 15C-CE, and 12-SE) and from four patients with low anti-T. cruzi antibody indices (3C-PB, 6C-PB, 16C-PB, and 17C-PB) at different dilutions: 1:50, 1:100, 1:250, 1:500, 1:1000 for 1 h at 37° C. After this period, the wells were washed three times with PBS-T and incubated with alkaline phosphatase-labeled human IgG antibody at a dilution of 1:5000 for 1 h. The wells were washed again three times with PBS-T buffer and the substrate para-nitrophenylphosphate (PNPP, 1 mg/mL, ThermoFischer SCIENTIFIC) was added. After 30 minutes, the absorbance was measured in an ELISA plate reader at 405 nm.

The results showed that for sera with low antibody titers, reading signals at dilutions 1:50, 1:100 and 1:250 were unequivocally above the threshold reached by the negative control, evidencing the potential of the PlatCruzi platform for detection of anti-T. cruzi antibodies in both high and low antibody patient sera.

The use of patient serum samples for experimental purposes was approved by the Ethics Committee of Fiocruz, as per authorization CEP/IOC—CAAE: 52892216.8.0000.5248.

### Example 5—Sensitivity and Specificity of PlatCruzi ELISA

The Elisa plates developed in the previous example containing 500 ng (0.3 M Urea, pH 8.0) of PlatCruzi were incubated with 71 sera from patients diagnosed for T. cruzi, plus 18 sera from patients diagnosed for leishmaniasis (negative for T. cruzi), 20 sera diagnosed for dengue (negative for T. cruzi), and 39 negative sera (other infections and uninfected individuals) at a dilution of 1:250 for 1 h at 37° C. After this period, the plates were subjected to washing and antibody labeling, developing and reading processes as previously performed.

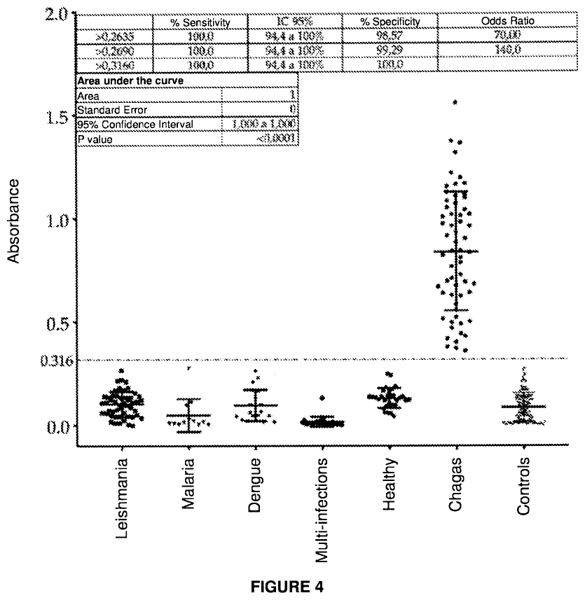

The results pointed to excellent sensitivity and specificity using the PlatCruzi platform (FIG. 4) from the receiver operating characteristic (ROC) curve correlation analysis. No false negative results were observed for the sera previously identified as positive for T. cruzi; as well as no false positives were observed for the other sera known to be negative for T. cruzi, including those positive for other infectious diseases. Both the sensitivity and specificity indices were 100%.

### Example 6—Development of RxRabies2 Protein

The “Rx” protein was tested for its performance and ability to express epitopes from other microorganisms, including viruses. The literature points to a significant number of specific polyamino acid sequences that can be used as targets for neutralizing antibodies. However, small variations in the sequences observed between viral strains can interfere with neutralization. Thus, a thorough study of the best polyamino acid sequences to use requires a great deal of knowledge of the biology of the virus and the epidemiology of its interaction with its host.

Rabies virus polyamino acid sequences were selected from the available state-of-the-art literature, considering experimental data on specificity and sensitivity for the diagnosis of the disease caused by rabies virus (Kuzmina et al., J Antivir Antiretrovir 5:2: 37-43, 2013; Cai et al, Microbes Infect 12: 948-955, 2010).

Ten polyamino acid sequences were selected for insertion into the ten insertion sites in the Rx protein, here called RaEp1 to RaEp10, as below. Combining the sequence of these polyamino acid sequences with the Rx protein gave rise to the RxRabies2 protein. The gene corresponding to the RxRabies2 protein, here called the RxRabies2 gene, is described in the SEQ ID NO nucleotide sequence: 19. The amino acid sequence corresponding to the RxRabies2 gene which contains the polyamino acid sequences RaEp1 to RaEp10, is described in SEQ ID NO: 20.

TABLE 6

| Polyamino acid | Position in | Original | | |
|---|---|---|---|---|
| sequences | the protein | epitope protein | Sequence | SEQ ID no. |
| RaEp 1 | 1 | antigenic site 1 | CKLKLCGVLGL | SEQ ID no. 21 |
| RaEp 2 | 2 | antigenic site 1 | CKLKLCGCSGL | SEQ ID no. 22 |
| RaEp 3 | 3 | antigenic site 1 | CKLKLCGVPGL | SEQ ID no. 23 |
| RaEp 4 | 4 | — | VDERGLYK | SEQ ID no. 24 |
| RaEp 5 | 5 | — | WVAMQTSN | SEQ ID no. 25 |
| RaEp 6 | 6 | antigenic site III | KSVRTWNEI | SEQ ID no. 26 |
| RaEp 8 | 8 | g5 antigenic site | LHDFHSD | SEQ ID no. 27 |
| RaEp 9 | 9 | g5 antigenic site | LHDFRSD | SEQ ID no. 28 |
| RaEp 10 | 10 | g5 antigenic site | LHDLHSD | SEQ ID no. 29 |

A synthetic gene containing a sequence coding for the Rx protein and sequences coding for the polyamino acid sequences described in Table 6 above has been synthesized.

The synthetic gene was introduced into pET28a plasmids using the restriction sites for the enzymes NdeI and XhoI by state-of-the-art molecular biology methods. In order to identify whether the synthetic gene matched the sequence designed for RxRabies2, a DH5α strain of Escherichia coli was transformed and the plasmid material analyzed by restriction enzyme digestion and then sequencing techniques, performed in the same way as described in PlatCruzi.

The analyzed plasmid harboring the correct sequence for RxRabies2 was transferred to E. coli, strain BL21, in order to produce the RxRabies2 protein. The BL21 strain can express T7 RNA polymerase when induced by Isopropyl β-D-1-thiogalactopyranoside (IPTG). The BL21 strain was grown overnight in LB medium and then reseeded in the same medium with kanamycin (30 μg/ml) on a shaker at 200 rpm until it reached an optical turbidity density of 0.6-0.8 (600 nm). Then, IPTG (q.s.p. 1 mM) is added to the culture and the same culture conditions are maintained for another 3 h at 37° C.

The culture was subjected to centrifugation and the pellet resuspended in urea buffer (100 mM NaH2PO4, 10 mM Tris-base, 8 M urea, pH 8.0). The solution was subjected to HisTrap™ affinity column chromatography, 1 mL, GE Healthcare Life Sciences, which allows high-resolution purification of histidine-tagged proteins, at a flow rate of 0.5 mL per minute, previously equilibrated in buffer A (50 mM Tris-HCl, pH 8.0, 100 mM NaCl and 5 mM imidazole). After binding, the resin was washed with 10 mL of buffer A. The protein was eluted in a 100% gradient of buffer B (50 mM Tris-HCl, pH 8.0, 100 mM NaCl, and 500 mM imidazole) at a flow rate of 0.7 mL/min for 45 minutes.

RxRabies2 production was analyzed in three different culture volumes; 3, 25 and 50 ml. As seen in Table 7 (below), the expression of RxRabies2 was 123 μg/ml on average.

TABLE 7

Amount of purified protein (μg/mL)

| | 3 mL | 25 | 50 | |
|---|---|---|---|---|
| Protein | culture | mL culture | mL culture | Average |
| RxRabies2 | 190 | 132 | 46 | 123 |

### Example 7—RxRabies2 Protein as Vaccine Composition

The RxRabies2 protein was produced as described in the previous example. 100 μg of protein was suspended in Freud's incomplete adjuvant (0.5 mL) and inoculated, intramuscularly, into the quadriceps of two 6-month-old male New Zealand rabbits. Rabbits were re-inoculated seven and fourteen days after the initial inoculation with RxRabies protein suspended in PBS (100 μg/0.5 mL).

After 21 days of inoculation of the first dose of the vaccine composition, blood was collected from the animals Plasma was collected by centrifugation and subjected to affinity purification by binding to Rx-Rabies2 protein adsorbed to the surface of a nitrocellulose membrane.

The nitrocellulose membrane containing the Rx-Rabies2 protein was prepared as described below in order to isolate it from potential contaminants After electrophoresis, the proteins were transferred to a nitrocellulose membrane using state-of-the-art Western blot techniques.
preparation of the 11% SDS-PAGE gel, as described in the previous example and in Table 5;
application of 10 μg of the Rx-Rabies2 protein on an 11% SDS-PAGE gel (Table 5) and submission to an electrophoretic current of 100 volts for approximately 2 hours;
transfer of proteins to nitrocellulose membrane: proteins were transferred to nitrocellulose membranes using Trans-Blot Cell (Bio-Rad, USA) with transfer buffer (25 mM Tris base, 192 mM glycine and 20% methanol) for one hour at 100V;
staining with Ponceau S red (Ponceau S 0.1%, acetic acid 5%) to confirm the presence of the recombinant protein.

The membrane was cut so as to specifically obtain a piece with only the RxRabies2 protein;
the membranes were then decolored in distilled water and left in TBS (0.1%) for 12 to 18 hours (overnight);
incubation with blocking solution (25 mM Tris-HCl, 125 mM NaCl pH 7.4 (TBS) containing 0.05% (v/v) Tween 20 (TBS-T) and 5% (w/v) skim milk dehydrate) overnight, then the membranes were incubated in blocking solution again for one hour and then three washes with TBS-T for 5 minutes each and three more 5-minute washes with TBS.

Sera from the immunized rabbits were diluted 1:500 in TBS and 10 ml were placed in contact with the nitrocellulose membrane segments containing the RxRabies2 protein for 1 h under stirring and then washed three times for 5 min with TBS-T and again washed 3 times for 5 min with TBS. Then, the specifically bound antibodies were released by adding 1 ml of 100 mM glycine (pH 3.0). The pH of the solution was raised to 7 by adding 100 μl of 1M Tris (pH 9.0) to purify the rabbit antibodies. The purification of antibodies that bind specifically to antigens is an important step in using these antibodies for therapy, as it allows for a drastic reduction in the amount to be administered while minimizing potential adverse effects.

Different extracts were used to demonstrate the specific ability of the produced rabbit antibodies to bind to RxRabies2. The following were used:
crude bacterial extract with Rx protein expression, obtained using the same conditions as mentioned in PlatCruzi;
rxRabies2 protein in the crude bacterial extract and purified at 1× and 0.5× concentrations;
purified Platcruzi protein at 1× and 0.5× concentrations.

The potential ligands were subjected to polyacrylamide gel electrophoresis (11% SDS-PAGE, Table 5), as described previously, and then transferred to nitrocellulose membrane and subjected to Western blot, the details of which have already been described for RxRabies2.

The membranes were incubated for one hour with the purified anti-RxRabies2 serum as described above, under agitation. Then three 5-minute washes with TBS-T and three 5-minute washes with TBS were done again. Subsequently, the membranes were incubated for one hour with the secondary anti-rabbit IgG antibodies with peroxidase diluted at 1:10,000. After incubation with the secondary antibody, three washes with TBS-T for 5 minutes and three washes with TBS for 5 minutes were performed. The development was performed with the aid of the SigmaFast™ DAB Peroxidase Substrate Tablet.

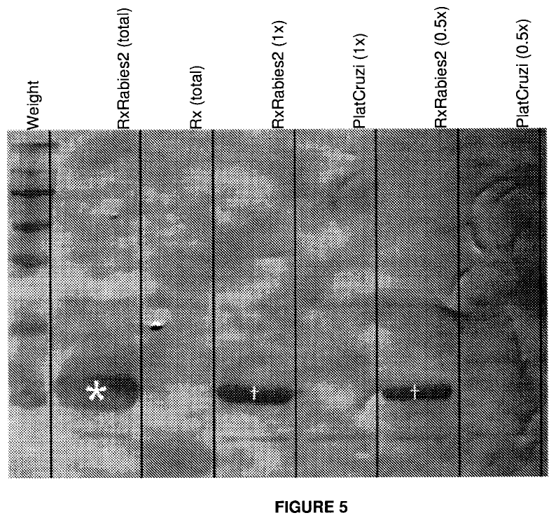

The results showed the specific binding of rabbit antibodies produced by inoculating RxRabies2 to ligands containing rabies virus 2 proteins. FIG. 5 shows read only bands in lanes 2, 4 and 6 which contain the crude bacterial extract containing the RxRabies2 protein, and the purified and diluted RxRabies2 protein at 1× and 0.5× concentrations, respectively.

The specificity of the immune response can also be observed by the lack of banding in lines 3, 5 and 7, containing (i) the Rx receptacle protein without epitope introduction, (ii) the Platcruzi platform, diluted 1× and 0.5×, respectively. These results confirm that the immune response was restricted to rabies virus epitopes, indicating that the Rx protein per se is not immunogenic.

### Example 8—Development of RxHoIgG3 Protein

From mapping studies of polyamino acid sequences of horse immunoglobulins (DeSimone et al., Toxicon 78: 83-93, 2014; Wagner et al, Journal of Immunology 173: 3230-3242, 2004), a sequence of horse IgG3 polyamino acids recognized by human IgG and IgE, suitable for use in laboratory assays to diagnose horse serum allergy, was identified.

The polyamino acid sequence DVLFTWYVDGTEV (SEQ ID NO: 30) was incorporated into the Rx protein at positions 1, 5, 6, 8, 9 and 10, giving rise to the RxHoIgG3 protein. The amino acid sequence of the RxHoIgG3 protein is described in SEQ ID NO: 31. The nucleotide sequence of the RxHoIgG3 protein is described in SEQ ID NO: 32. A synthetic gene containing a sequence coding for the Rx protein and the sequence coding for the polyamino acid sequence described above (SEQ ID NO: 30) has been synthesized.

The synthetic gene was introduced into pET28a plasmids using the restriction sites for the enzymes NdeI and XhoI by state-of-the-art molecular biology methods. In order to identify whether the synthetic gene matched the sequence designed for the RxHoIgG3 protein, a DH5α strain of Escherichia coli was transformed and the plasmid material analyzed by restriction enzyme digestion techniques and then sequenced, as already specified in PlatCruzi.

The analyzed plasmid harboring the correct sequence for RxHoIgG3 was transferred to E. coli, strain BL21, in order to produce the RxHoIgG3 protein. The BL21 strain can express T7 RNA polymerase when induced by Isopropyl β-D-1-thiogalactopyranoside (IPTG). The BL21 strain was grown overnight in LB medium and then reseeded in the same medium with kanamycin (30 μg/ml) on a shaker at 200 rpm until it reached an optical turbidity density of 0.6-0.8 (600 nm). Then, IPTG (q.s.p. 1 mM) is added to the culture and the same culture conditions are maintained for another 3 h.

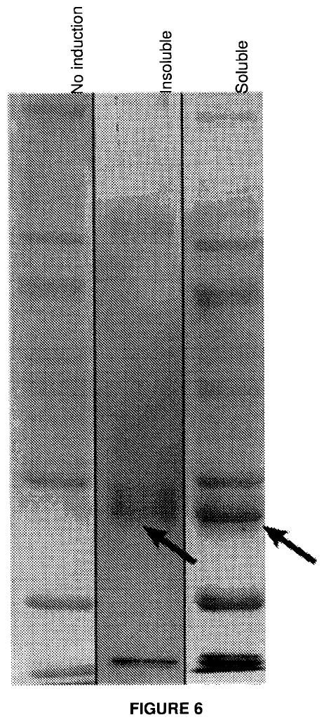

The culture was subjected to centrifugation and the pellet resuspended in urea buffer (100 mM NaH2PO4, 10 mM Tris-base, 8 M urea, pH 8.0). The solution was chromatographed using a nickel affinity column (HisTrap™, 1 mL, GE Healthcare Life Sciences) and flowed at 0.5 mL per minute, previously equilibrated in buffer A (50 mM Tris-HCl, pH 8.0, 100 mM NaCl and 5 mM imidazole). After binding, the resin was washed with 10 mL of buffer A. The protein was eluted in a 100% gradient of buffer B (50 mM Tris-HCl, pH 8.0, 100 mM NaCl, and 500 mM imidazole) at a flow rate of 0.7 mL/min for 45 minutes. The production of RxHoIgG3 protein can be evidenced in the eluate after 11% SDS-PAGE electrophoresis (Table 5). The results are presented in FIG. 6, and show the expression of RxHoIgG3 as a recombinant protein. No bands were detected in the uninduced soluble bacterial extract (FIG. 6, column 1), and one each in the insoluble (column 2) and soluble (column 3) fractions.

### Example 9—Development of RxOro Protein

From a polyamino acid sequence mapping study of oropouche virus (strain Q71MJ4 and Q9J945, Uniprot) (Acrani et al, Journal of General Virology 96: 513-523, 2014 Tilston-Lunel et al, Journal of General Virology 96 (Pt 7): 1636-1650, 2015), we selected polyamino acid sequences from spot synthesis or peptide microarray techniques available in the state of the art, considering their diagnostic potential. Six polyamino acid sequences were selected for insertion into nine insertion points in the Rx protein, herein called OrEp 1 to OrEp 7, as shown in table 8 below:

TABLE 8

| Polyamino | Position in | Original | | |
|---|---|---|---|---|
| acid sequences | the protein | epitope protein | Sequence | SEQ ID no. |
| OrEp 1 | 1 | G1 | YIEKDDSDALKALF | SEQ ID no. 35 |
| OrEp 2 | 3 | G2 | GNFMVLSVDD | SEQ ID no. 36 |
| OrEp 2 | 4 | G2 | GNFMVLSVDD | SEQ ID no. 36 |
| OrEp 3 | 5 | N | KTSRPMVDLTFGGVQ | SEQ ID no. 37 |
| OrEp 3 | 6 | N | KTSRPMVDLTFGGVQ | SEQ ID no. 37 |
| OrEp 4 | 7 | N | IFNDVPQRTTSTFDP | SEQ ID no. 38 |
| OrEp 4 | 8 | N | IFNDVPQRTTSTFDP | SEQ ID no. 38 |
| OrEp 5 | 9 | G2 | LYSDLFSKNLVTEY | SEQ ID no. 39 |
| OrEp 6 | 10 | G1 | YIEKDDSDALKALF | SEQ ID no. 40 |

The combination of these polyamino acid sequences described above with the Rx protein gave rise to the RxOro protein. The amino acid sequence corresponding to the RxOro gene, containing the polyamino acid sequences OrEp1 to OrEp6, is described in SEQ ID no. 33. The gene corresponding to the RxOro protein, herein referred to as the RxOro gene, is described in SEQ ID nucleotide sequence no. 34.

A synthetic RxOro gene was synthesized and introduced into pET28a plasmids using the restriction sites for the enzymes NdeI and XhoI by state-of-the-art molecular biology methods. In order to identify whether the synthetic gene matched the sequence designed for RxOro, a DH5α strain of Escherichia coli was transformed and the plasmid material analyzed by restriction enzyme digestion and subsequently sequencing techniques as described in PlatCruzi.

The analyzed plasmid harboring the correct sequence for the RxOro protein was transferred to E. coli, strain BL21. The BL21 strain can express T7 RNA polymerase when induced by Isopropyl β-D-1-thiogalactopyranoside (IPTG). The BL21 strain was grown overnight in LB medium and then reseeded in the same medium with kanamycin (30 μg/ml) on a shaker at 200 rpm until it reached an optical turbidity density of 0.6-0.8 (600 nm). Then, IPTG (q.s.p. 1 mM) is added to the culture and the same culture conditions are maintained for another 3 h.

The culture was subjected to centrifugation and the pellet resuspended in urea buffer (100 mM NaH2PO4, 10 mM Tris-base, 8 M urea, pH 8.0). The solution was chromatographed using a nickel affinity column (HisTrap™, 1 mL, GE Healthcare Life Sciences) and flowed at 0.5 mL per minute, previously equilibrated in buffer A (50 mM Tris-HCl, pH 8.0, 100 mM NaCl and 5 mM imidazole). After binding, the resin was washed with 10 mL of buffer A. The protein was eluted in a 100% gradient of buffer B (50 mM Tris-HCl, pH 8.0, 100 mM NaCl, and 500 mM imidazole) at a flow rate of 0.7 mL/min for 45 minutes. RxOro production was examined in three different volumes of bacterial growth; 3, 25 and 50 ml. The expression level of RxOro was 203 μg/ml on average and the results are shown in Table 9.

TABLE 9

Amount of purified protein (μg/mL)

| | 3 mL | 25 | 50 | |
|---|---|---|---|---|
| Protein | culture | mL culture | mL culture | Average |
| RxOro | 407 | 164 | 40 | 204 |

### Example 10—Development of an ELISA from RxOro

The performance of the RxOro protein was evaluated against a panel of sera from individuals affected by Oropouche infections. RxOro protein, in solution (0.3 M Urea, pH 8.0), was added to a 96-well ELISA plate in the amount of 500 ng/orifice at 4° C. for 12-18 h. The wells were washed with saline-phosphate buffer (PBS) solution added Tween 20 (PBS-T, 10 mM sodium phosphate—Na3PO4, 150 mM sodium chloride—NaCl and 0.05% Tween-20, pH 7.4) and then incubated with 1×PBS buffer containing 5% (weight/volume) skim milk dehydrate for 2 h at 37° C.

The wells were then washed 3 times with PBS-T buffer and incubated with 98 sera samples from patients suspected of Oropouche virus infection and 51 sera samples from healthy patients diluted 1:100, for 1 h at 37° C. After the incubation period, the wells were washed three times with PBS-T and then incubated with alkaline phosphatase-labeled human IgG antibody at 1:5000 dilution for 1 h at 37° C.

The wells were washed again three times with PBS-T buffer and the substrate para-nitrophenylphosphate (PNPP, 1 mg/mL, ThermoFischer SCIENTIFIC) was added. After 30 minutes and under shelter from light, the absorbance was measured in an ELISA plate reader at 405 nm.

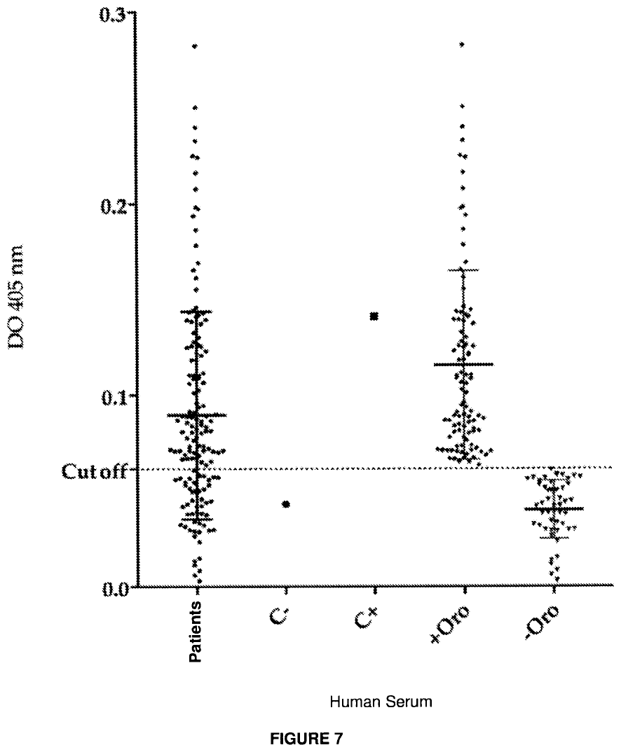

The results pointed to excellent sensitivity and specificity using RxOro (FIG. 7). The results strongly support the use of RxOro for detection of Oropouche virus infections.

The use of patient serum samples for experimental purposes was approved by the Ethics Committee of Fiocruz, as per authorization CEP/IOC—CAAE: 52892216.8.0000.5248.

### Example 11—Development of RxMayaro_IgG Protein

From a mapping study of polyamino acid sequences of Mayaro virus (strain Q8QZ73 and Q8QZ72, Uniprot) (Espósito et al., Genome Announcement 3: e01372-15, 2015), were selected polyamino acid sequences from the available state-of-the-art literature, considering their diagnostic potential. Four polyamino acid sequences were selected for insertion into nine insertion points in the Rx protein, here named MGEp1 to MGEp 4, as shown in Table 10 below:

TABLE 10

| Polyamino acid | Position in | Original epitope | | |
|---|---|---|---|---|
| sequences | the protein | protein | Sequence | SEQ ID no. |
| MGEp 1 | 1 | nsP2 | KLSATDWSAI | SEQ ID no. 41 |
| MGEp 2 | 3 | Capsid | KPKPQPEK | SEQ ID no. 42 |
| MGEp 3 | 4 | nsP1 | KKMTPSDQI | SEQ ID no. 43 |
| MGEp 4 | 5 | nsP3 | VELPWPLETI | SEQ ID no. 44 |
| MGEp 2 | 6 | Capsid | KPKPQPEK | SEQ ID no. 42 |
| MGEp 1 | 7 | nsP2 | KLSATDWSAI | SEQ ID no. 41 |
| MGEp 4 | 8 | nsP3 | VELPWPLETI | SEQ ID no. 44 |
| MGEp 3 | 9 | nsP1 | KKMTPSDQI | SEQ ID no. 43 |
| MGEp 1 | 10 | nsP2 | KLSATDWSAI | SEQ ID no. 41 |

Combining the sequence of these polyamino acid sequences described above with the Rx protein gave rise to the RxMayaro_IgG protein. The amino acid sequence corresponding to the RxMayaro_IgG gene, containing the polyamino acid sequences MGEp 1 a MGEp 4 is described in SEQ ID no. 45. The gene corresponding to the RxMayaro_IgG protein, herein referred to as the RxMayaro_IgG gene, is described in SEQ ID nucleotide sequence no. 46.

A synthetic RxMayaro_IgG gene was synthesized and introduced into pET28a plasmids using the restriction sites for the enzymes NdeI and XhoI via molecular biology methods known to the state of the art. In order to identify whether the synthetic gene matched the sequence designed for RxMayaro_IgG, a DH5α strain of Escherichia coli was transformed and the plasmid material analyzed by restriction enzyme digestion techniques and then sequenced, as cited in PlatCruzi.

The analyzed plasmid harboring the correct sequence for the RxMayaro_IgG protein was transferred into E. coli, strain BL21. The BL21 strain can express T7 RNA polymerase when induced by Isopropyl β-D-1-thiogalactopyranoside (IPTG). The BL21 strain was grown overnight in LB medium and then reseeded in the same medium with kanamycin (30 μg/ml) on a shaker at 200 rpm until it reached an optical turbidity density of 0.6-0.8 (600 nm). Then, IPTG (q.s.p. 1 mM) is added to the culture and the same culture conditions are maintained for another 3 h.

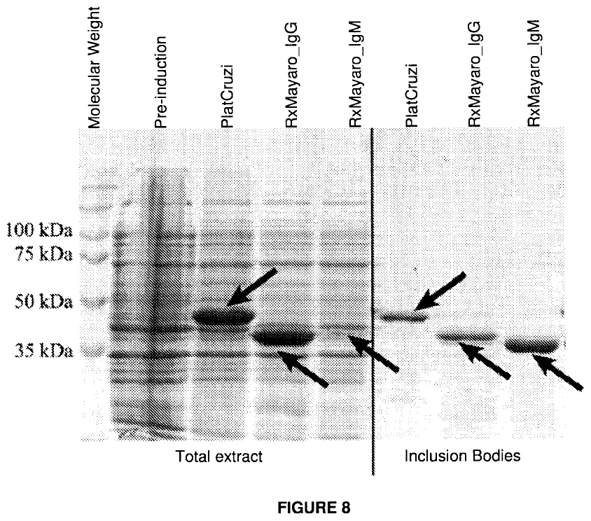

The culture was subjected to centrifugation and the pellet resuspended in urea buffer (100 mM NaH2PO4, 10 mM Tris-base, 8 M urea, pH 8.0). The solution was chromatographed on a nickel affinity column (HisTrap™, 1 mL, GE Healthcare Life Sciences) at a flow rate of 0.5 mL per minute, previously equilibrated in buffer A (50 mM Tris-HCl, pH 8.0, 100 mM NaCl and 5 mM imidazole). After binding, the resin was washed with 10 mL of buffer A. The protein was eluted in a 100% gradient of buffer B (50 mM Tris-HCl, pH 8.0, 100 mM NaCl, and 500 mM imidazole) at a flow rate of 0.7 mL/min for 45 minutes. Production of the RxMayaro_IgG protein can be evidenced in the eluate from performing 11% SDS-PAGE electrophoresis (Table 5). The RxMayaro_IgG protein was also examined by SDS-PAGE to confirm its production and determine its distribution between the soluble and insoluble fractions. As seen in FIG. 8, columns 4 and 7 (arrows) show that RxMayaro_IgG is produced as soluble and insoluble.

RxMayaro_IgG production was examined at three different growth volumes: 3, 25 and 50 ml. As seen in Table 11 (below), the expression level of RxMayaro_IgG was 130 μg/ml on average.

TABLE 11

| | Amount of purified protein (μg/mL) | | | |
|---|---|---|---|---|
| | 3 mL | 25 | 50 | |
| Protein | culture | mL culture | mL culture | Media |
| RxMayaro_IgG | 157 | 168 | 64 | 130 |

### Example 12—Development of ELISA from RxMayaro_IgG

The performance of the RxMayaro_IgG protein was evaluated against a panel of sera from individuals affected by Mayaro virus infections. RxMayaro_IgG protein, in solution (0.3 M Urea, pH 8.0), was added to a 96-hole ELISA plate in the amount of 500 ng/orifice at 4° C. for 12-18 h. The wells were washed with saline-phosphate buffer (PBS) solution added Tween 20 (PBS-T, 10 mM sodium phosphate—Na3PO4, 150 mM sodium chloride—NaCl and 0.05% Tween-20, pH 7.4) and then incubated with 1×PBS buffer containing 5% (weight/volume) dehydrated skim milk for 2 h at 37° C.

The wells were then washed three times with PBS-T buffer and incubated with 6 samples of sera from patients suspected of Mayaro virus infection and 29 samples of sera from healthy patients diluted 1:100, for 1 h at 37° C. After the incubation period, the wells were washed three times with PBS-T and then incubated with alkaline phosphatase-labeled human IgG antibody at a dilution of 1:5000, for 1 h at 37° C. The wells were washed again three times with PBS-T buffer and the substrate para-nitrophenylphosphate (PNPP, 1 mg/mL, ThermoFischer SCIENTIFIC) was added. After 30 minutes and under shelter from light, the absorbance was measured in an ELISA plate reader at 405 nm.

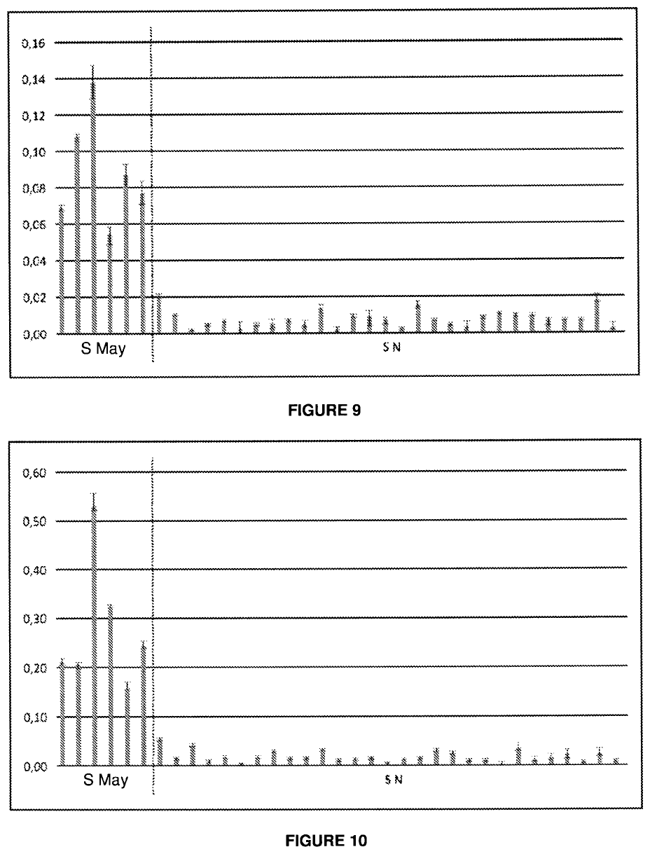

The results pointed to excellent sensitivity and specificity using RxMayaro_IgG (FIG. 9). The results strongly support the use of RxMayaro_IgG for detection of Mayaro virus infections.

The use of patient serum samples for experimental purposes was approved by the Ethics Committee of Fiocruz, as per authorization CEP/IOC—CAAE: 52892216.8.0000.5248.

### Example 13—Development of RxMayaro_IgM Protein

From a mapping study of polyamino acid sequences of Mayaro virus (strain Q8QZ73 and Q8QZ72, Uniprot) (Espósito et al., Genome Announcement 3: e01372-15, 2015), were selected polyamino acid sequences from the available state-of-the-art literature, considering their diagnostic potential. Four polyamino acid sequences were selected for insertion into nine insertion points in the Rx protein, herein named MMEp1 to MMEp 4, as shown in table 12 (below):

TABLE 12

| Polyamino acid | Position in | Original epitope | | |
|---|---|---|---|---|
| sequences | the protein | protein | Sequence | SEQ ID no. |
| MMEp1 | 1 | nsP1 | HRIRLLLQS | SEQ ID no. 47 |
| MMEp2 | 3 | E2 | SYRTGAERV | SEQ ID no. 48 |
| MMEp3 | 4 | nsP2 | NGVKQTVDV | SEQ ID no. 49 |
| MmEp4 | 5 | E1 | QSRTLDSRD | SEQ ID no. 50 |
| MMEp 4 | 6 | E1 | QSRTLDSRD | SEQ ID no. 50 |
| MMEp1 | 7 | nsP1 | HRIRLLLQS | SEQ ID no. 47 |
| MMEp 2 | 8 | E2 | SYRTGAERV | SEQ ID no. 48 |
| MMEp3 | 9 | nsP2 | NGVKQTVDV | SEQ ID no. 49 |
| MMEp1 | 10 | nsP1 | HRIRLLLQS | SEQ ID no. 47 |

Combining the sequence of these polyamino acid sequences described above with the Rx protein gave rise to the RxMayaro_IgM protein. The amino acid sequence corresponding to the RxMayaro_IgM gene, containing the polyamino acid sequences MMEp 1 a MMEp 4 is described in SEQ ID no. 51. The gene corresponding to the RxMayaro_IgM protein, herein referred to as the RxMayaro_IgM gene, is described in SEQ ID nucleotide sequence no. 52.

A synthetic RxMayaro_IgM gene was synthesized and introduced into pET28a plasmids using the restriction sites for the enzymes NdeI and XhoI via molecular biology methods known to the state of the art. In order to identify whether the synthetic gene matched the sequence designed for RxMayaro_IgM, a DH5α strain of Escherichia coli was transformed and the plasmid material analyzed by restriction enzyme digestion techniques and then sequenced, as previously described in PlatCruzi.

The analyzed plasmid harboring the correct sequence for the RxMayaro_IgM protein was transferred into E. coli, strain BL21. The BL21 strain can express T7 RNA polymerase when induced by Isopropyl β-D-1-thiogalactopyranoside (IPTG). The BL21 strain was grown overnight in LB medium and subsequently reseeded in the same medium, with kanamycin (30 μg/ml) added, on a shaker at 200 rpm until it reached an optical turbidity density of 0.6-0.8 (600 nm). Then, IPTG (q.s.p. 1 mM) is added to the culture and the same culture conditions are maintained for another 3 h.

The culture was subjected to centrifugation and the pellet resuspended in urea buffer (100 mM NaH2PO4, 10 mM Tris-base, 8 M urea, pH 8.0). The solution was chromatographed on a nickel affinity column (HisTrap™, 1 mL, GE Healthcare Life Sciences) at a flow rate of 0.5 mL per minute, previously equilibrated in buffer A (50 mM Tris-HCl, pH 8.0, 100 mM NaCl and 5 mM imidazole). After binding, the resin was washed with 10 mL of buffer A. The protein was eluted in a 100% gradient of buffer B (50 mM Tris-HCl, pH 8.0, 100 mM NaCl, and 500 mM imidazole) at a flow rate of 0.7 mL/min for 45 minutes.

The production of RxMayaro_IgM protein can be evidenced in the eluate by performing SDS-PAGE electrophoresis and its distribution can be observed between the soluble and insoluble fractions. As can be seen in FIG. 8, columns 5 and 8 (arrows) show that RxMayaro_IgM is produced as soluble and insoluble.

RxMayaro_IgM production was also examined at three different growth volumes: 3, 25 and 50 ml. As seen in Table 13 (below), the expression level of RxMayaro_IgM was 205 μg/ml on average.

TABLE 13

| | Amount of purified protein (μg/mL) | | | |
|---|---|---|---|---|
| | 3 mL | 25 | 50 | |
| Protein | culture | mL culture | mL culture | Average |
| RxMayaro_IgM | 200 | 172 | 242 | 205 |

### Example 14—Development of an ELISA Based on RxMayaro_IgM

The performance of the RxMayaro_IgM protein was evaluated against a panel of sera from individuals affected by Mayaro virus infections. RxMayaro_IgM protein, in solution (0.3 M Urea, pH 8.0), was added to a 96-hole ELISA plate in the amount of 500 ng/orifice at 4° C. for 12-18 h. The wells were washed with saline-phosphate buffer (PBS) solution added Tween 20 (PBS-T, 10 mM sodium phosphate—Na3PO4, 150 mM sodium chloride—NaCl and 0.05% Tween-20, pH 7.4) and then incubated with 1×PBS buffer containing 5% (weight/volume) dehydrated skim milk for 2 h at 37° C.

The wells were then washed three times with PBS-T buffer and incubated with 6 sera samples from patients suspected of Mayaro virus infection and 29 sera samples from healthy patients diluted 1:100, for 1 h at 37° C. After the incubation period, the wells were washed three times with PBS-T and then incubated with alkaline phosphatase-labeled human IgM antibody at a dilution of 1:5000, for 1 h at 37° C. The wells were washed again three times with PBS-T buffer and the substrate para-nitrophenylphosphate (PNPP, 1 mg/mL, ThermoFischer SCIENTIFIC) was added. After 30 minutes and under shelter from light, the absorbance was measured in an ELISA plate reader at 405 nm.

The results pointed to excellent sensitivity and specificity using RxMayaro_IgM (FIG. 10). The results strongly support the use of RxMayaro_IgM for detection of Mayaro virus infections.

The use of patient serum samples for experimental purposes was approved by the Ethics Committee of Fiocruz, as per authorization CEP/IOC—CAAE: 52892216.8.0000.5248.

### Example 15—Protein RxPtx Development

From a mapping study of polyamino acid sequences of the bacterial toxin protein Bordetella pertussis (P04977; P04978; P04979; P0A3R5 and P04981: Uniprot), causing pertussis, polyamino acid sequences were selected from the available state-of-the-art literature, considering their diagnostic potential. Ten polyamino acid sequences were selected for insertion into nine insertion sites in the Rx protein, here called PtxEp1 to PtxEp 10, as shown in Table 14 below. In this example, two epitopes were located at position 1 using a spacer (SEQ ID NO: 95: SYWKGS) among them. Two epitopes were also inserted at position 10 using another spacer (SEQ ID NO: 96: EAAKEAAK). The purpose of introducing these spacers is to generate an inert physical space between consecutive polyaminoacids, thus helping to prevent binding competition between adjacent antibodies.

TABLE 14

| Polyamino | Position in | Original | | |
|---|---|---|---|---|
| acid sequences | the protein | epitope protein | Sequence | SEQ ID no. |
| PtxEp 1 | 1 | Ptx-S1 | PYTSRRSVASIVGT | SEQ ID no. 53 |
| Spacer | Between PtxEp1 | AT | SYWKGS | SEQ ID no. 95 |
| | and 2 | | | |
| PtxEp 2 | 1 | Ptx-S3 | QYYDYEDATF | SEQ ID no. 54 |
| PtxEp 3 | 2 | Ptx-S4 | GPKQLTFEGK | SEQ ID no. 55 |
| PtxEp 4 | 3 | Ptx-S2 | DATFETYALT | SEQ ID no. 56 |
| PtxEp 5 | 5 | Ptx-S5 | LTVEDSPYP | SEQ ID no. 57 |
| PtxEp 6 | 6 | Ptx-S1 | ALATYQSEY | SEQ ID no. 58 |
| PtxEp 7 | 8 | Ptx-S3 | PGIVIPPKALFTQQQ | SEQ ID no. 59 |
| PtxEp 8 | 9 | Ptx-S1 | AVEAERAGR | SEQ ID no. 60 |
| PtxEp 9 | 10 | Ptx-S1 | TTTEYSNAR | SEQ ID no. 61 |
| Spacer | Between PtxEp9 | AT | EAAKEAAK | SEQ ID no. 96 |
| | and 10 | | | |
| PtxEp10 | 10 | Ptx-S1 | ERAGEAMVLVYYES | SEQ ID no. 62 |

Combining the sequence of these polyamino acid sequences described above with the Rx protein gave rise to the protein RxPtx. The amino acid sequence corresponding to the gene RxPtx gene, containing the epitopes PtxEp 1 a PtxEp 10 is described in SEQ ID NO: 64. The gene corresponding to the RxPtx gene, herein referred to as the RxPtx gene is described in the SEQ ID NO nucleotide sequence: 63.

A synthetic gene RxPtx was synthesized and introduced into pET28a plasmids using the restriction sites for the enzymes NdeI and XhoI by state-of-the-art molecular biology methods. In order to identify whether the synthetic gene matched the sequence designed for RxPtx a DH5α strain of Escherichia coli was transformed and the plasmid material analyzed by restriction enzyme digestion and subsequently sequencing techniques, as previously described in PlatCruzi.

The analyzed plasmid harboring the correct sequence for the RxPtx protein was transferred to E. coli, strain BL21. The BL21 strain can express T7 RNA polymerase when induced by Isopropyl β-D-1-thiogalactopyranoside (IPTG). The BL21 strain was grown overnight in LB medium and subsequently reseeded in the same medium, with kanamycin (30 μg/ml) added, on a shaker at 200 rpm until it reached an optical turbidity density of 0.6-0.8 (600 nm). Then, IPTG (q.s.p. 1 mM) is added to the culture and the same culture conditions are maintained for another 3 h.

The culture was subjected to centrifugation and the pellet resuspended in 2 mL of PBS with CelLytic (0.5×) for one hour at 4° C. After another centrifugation, the supernatant was collected and the pellet was resuspended in 8 M urea solution (pH 8.0) in the same volume as the supernatant. Equal volumes were loaded on the SDS-PAGE gel (11%, Table 5).

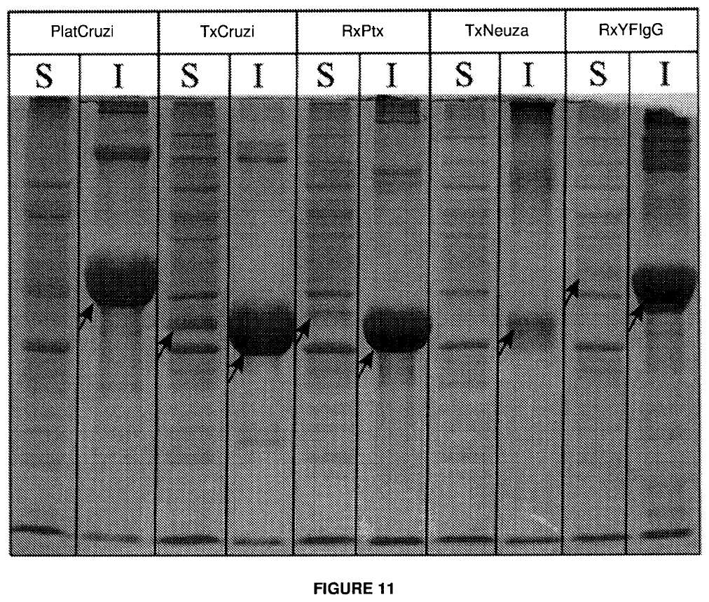

The RxPtx protein was examined by SDS-PAGE electrophoresis to confirm its production and determine its distribution between the soluble and insoluble fractions. As seen in FIG. 11, columns 5 and 6 (arrows) show that RxPtx is produced as soluble and insoluble with a higher proportion in the insoluble fraction.

### Example 16—Development of RxYFIgG Protein

From a mapping study of polyamino acid sequences of the yellow fever virus (strain 17DD and the sequences in the p03314-Uniprot archive)polyamino acid sequences were selected from the available state-of-the-art literature, considering their diagnostic potential. Ten polyamino acid sequences were selected for insertion into nine insertion sites in the Rx protein, here called YFIgGEp1 to YFIgGEp10, as shown in Table 15 below. Two epitopes were located at position 10 using a spacer (SEQ ID NO: 97: TSYWKGS) between them. The spacer has the function of creating a physical space between consecutive epitopes, helping to preserve the interaction with the antibodies.

TABLE 15

| Polyamino | Position in | Original | | |
|---|---|---|---|---|
| acid sequences | the protein | epitope protein | Sequence | SEQ ID no. |
| YFIgGEp 1 | 1 | NS4B | SPWSWPDLDLKPGA | SEQ ID no. 65 |
| YFIgGEp 2 | 3 | NS2A | DGNCDGRGKSTRST | SEQ ID no. 66 |
| YFIgGEp 3 | 4 | NS1 | VFSPGRKNGSFIID | SEQ ID no. 67 |
| YFIgGEp 4 | 5 | NS4B | HVQDCDESVLTRLE | SEQ ID no. 68 |
| YFIgGEp 5 | 6 | NS1 | DCDGSILGAAVNGK | SEQ ID no. 69 |
| YFIgGEp 6 | 7 | NS1 | FTTRVYMDA | SEQ ID no. 70 |
| YFIgGEp 7 | 8 | NS1 | RDSDDDWLNKYSYYP | SEQ ID no. 71 |
| YFIgGEp 8 | 9 | NS1 | ESEMFMPRSIGGPV | SEQ ID no. 72 |
| YFIgGEp 9 | 10 | NS4B | AEAEMVIHHQHVQD | SEQ ID no. 73 |
| Spacer | Between | | TSYWKGS | SEQ ID no. 97 |
| | YFIgGEp9 | | | |
| | and 10 | | | |
| YFIgGEp 10 | 10 | NS1 | LEHEMWRSRADEINAIFEE | SEQ ID no. 74 |

Combining the sequence of these polyamino acids described above with the Rx protein gave rise to the protein RxYFIgG protein. The amino acid sequence corresponding to the gene RxYFIgG containing the epitopes YFIgGEp 1 a YFIgGEp 10 is described in SEQ ID NO: 75. The gene corresponding to the RxYFIgG protein gene, herein called the RxYFIgG gene is described in SEQ ID nucleotide sequence no. 76.

A synthetic gene RxYFIgG was synthesized and introduced into pET28a plasmids using the restriction sites for the enzymes NdeI and XhoI by state-of-the-art molecular biology methods. In order to identify whether the synthetic gene matched the sequence designed for RxYFIgG, a DH5α strain of Escherichia coli was transformed and the plasmid material analyzed by restriction enzyme digestion and subsequently sequencing techniques, as previously described in PlatCruzi.

The analyzed plasmid harboring the correct sequence for RxYFIgG was transferred to E. coli, strain BL21, in order to produce the RxYFIgG protein. The BL21 strain can express T7 RNA polymerase when induced by Isopropyl β-D-1-thiogalactopyranoside (IPTG). The BL21 strain was grown overnight in LB medium at 37° C. and subsequently reseeded in the same medium with kanamycin (30 μg/ml) on a shaker at 200 rpm until an optical turbidity density of 0.6-0.8 (600 nm) was reached. Then IPTG (q.s.p. 1 mM) is added to the culture and the same culture conditions are maintained for another 3 h at 37° C.

The culture was centrifuged and the pellet resuspended in 2 mL PBS with CelLytic in 2 mL of PBS with CelLytic (0.5×) for one hour at 4° C. After another centrifugation, the supernatant was collected and the pellet was resuspended in 8 M urea solution (pH 8.0) in the same volume as the supernatant. Equal volumes were loaded on the SDS-PAGE gel (11%, Table 5). The protein RxYFIgG was examined by SDS-PAGE electrophoresis to confirm its production and to determine its distribution between the soluble and insoluble fractions. As seen in FIG. 11, columns 9 and 10 (arrows) show that RxYFIgG is produced as both soluble and insoluble, with a higher proportion in the insoluble fraction.

### Example 17—Development of TxNeuza Protein

The receptacle protein “Tx” has been genetically manipulated to harbor epitopes of t-cell epitopes from Dermatophogoides pteronyssinus a leading cause of respiratory allergy in humans, which we refer to here as the TxNeuza platform. The gene corresponding to the TxNeuza protein, here called the TxNeuza gene, is described in the SEQ ID NO nucleotide sequence: 89.

From a mapping study of T-cell polyamino acid sequences from D. pteronyssinus, polyamino acid sequences were selected from the available state-of-the-art literature considering their diagnostic potential for allergies caused by D. pteronyssinus (Hinz et al., Clin Exp Allergy 45: 1601-1612, 2015; Oseroff et al, Clin Exp Allergy 47:577-592, 2017). Nine polyamino acid sequences were selected for insertion into nine insertion sites in the Tx protein, here named NeuzaEp1 to NeuzaEp9, as shown in Table 16 below. In this example, two epitopes were located at position 12 using a spacer (SEQ ID NO: 98: GGSG) among them.

TABLE 16

| Polyamino | Position in | Original | | |
|---|---|---|---|---|
| acid sequences | the protein | epitope protein | Sequence | SEQ ID no. |
| NeuzaEp1 | 12 | Derp1 | DLRQMRTVTPIRMQGGCGSC | SEQ ID no. 79 |
| NeuzaEp2 | 10 | Derp1 | GCGSCWAFSGVAATESAYLA | SEQ ID no. 80 |
| NeuzaEp3 | 7 | Derp1 | QESYYRYVAREQSCR | SEQ ID no. 81 |
| NeuzaEp4 | 2 | Derp1 | HAVNIVGYSNAQGVD | SEQ ID no. 82 |
| NeuzaEp5 | 9 | Derp2 | CHGSEPCIIHRGKPFQLEAV | SEQ ID no. 83 |
| NeuzaEp6 | 6 | Derp2 | YDIKYTWNVPKIAPKSENVVV | SEQ ID no. 84 |
| NeuzaEp7 | 12 | Derp2 | NTKTAKIEIKASIDG | SEQ ID no. 85 |
| NeuzaEp8 | 5 | Derp2 | GVLACAIATHAKIRD | SEQ ID no. 86 |
| NeuzaEp9 | 1 | Derp23 | PKDPHKFYICSNWEAVHKDC | SEQ ID no. 87 |
| Spacer | Between | | GGSG | SEQ ID no. 98 |
| | NeuzaEp1 | | | |
| | and 7 | | | |

Combining the sequence of these epitopes described above with the Tx protein gave rise to the TxNeuza protein. The amino acid sequence corresponding to the TxNeuza gene, containing the epitopes NeuzaEp 1 a NeuzaEp9 is described in SEQ ID NO: 88.

A synthetic gene TxNeuza was synthesized and introduced into pET28a plasmids using the restriction sites for the enzymes NdeI and XhoI by state-of-the-art molecular biology methods. In order to identify whether the synthetic gene matched the sequence designed for TxNeuza a DH5α strain of Escherichia coli was transformed and the plasmid material analyzed by restriction enzyme digestion and subsequently sequencing techniques, as previously described in PlatCruzi. [00210] The analyzed plasmid harboring the correct sequence for TxNeuza was transferred to E. coli, strain BL21, in order to produce the TxNeuza protein. The BL21 strain can express T7 RNA polymerase when induced by Isopropyl β-D-1-thiogalactopyranoside (IPTG). The BL21 strain was grown overnight in LB medium at 37° C. and subsequently reseeded in the same medium with kanamycin (30 μg/ml) on a shaker at 200 rpm until an optical turbidity density of 0.6-0.8 (600 nm) was reached. Then IPTG (q.s.p. 1 mM) is added to the culture and the same culture conditions are maintained for another 3 h at 37° C.

The culture was centrifuged and the pellet resuspended in 2 mL PBS with CelLytic in 2 mL of PBS with CelLytic (0.5×) for one hour at 4° C. After another centrifugation, the supernatant was collected and the pellet was resuspended in 8 M urea solution (pH 8.0) in the same volume as the supernatant. Equal volumes were loaded on the SDS-PAGE gel (11%, Table 5). The TxNeuza protein was examined by SDS-PAGE to confirm its production and determine its distribution between the soluble and insoluble fractions. As seen in FIG. 11, columns 7 and 8 (arrows) show that the TxNeuza protein is produced as insoluble.

### Example 18—Development of TxCruzi Protein

T. cruzi polyamino acid sequences were selected from the available state-of-the-art literature, considering experimental specificity and sensitivity data for diagnostic tests for Chagas disease (Balouz, et al., Clin Vaccine Immunol 22, 304-312, 2015; Alvarez, et al., Infect Immun 69, 7946-7949, 2001; Fernandez-Villegas, et al., J Antimicrob Chemother 71, 2005-2009, 2016; Thomas, et al., Clin Vaccine Immunol 19, 167-173, 2012). Ten polyamino acid sequences were selected for insertion into the ten insertion sites in the “Tx” protein, here called TcEp 1, TcEp 3, TcEp 4, TcEp 6, TcEp 8, TcEp 9, TcEp 10, TcEp 11, TcEp 12, and TcEp 13, as shown in Table 17 below. Two epitopes were located at position 12 using a spacer (SEQ ID NO: 99: GGASG) among them.

TABLE 17

| Polyamino | Position in | Original | | |
|---|---|---|---|---|
| acid sequences | the protein | epitope protein | Sequence | SEQ ID no. |
| TcEp1 | 1 | KMP11 | KFAELLEQQKNAQFPGK | SEQ ID no. 7 |
| TcEp11 | 4 | SAPA | DSSAHSTPSTPA | SEQ ID no. 92 |
| TcEp4 | 6 | PEP-2 | GDKPSPFGQAAAADK | SEQ ID no. 10 |
| TcEp12 | 7 | TcCA-2 | FGQAAAGDKPS | SEQ ID no. 93 |
| TcEp6 | 8 | TcD-1 | AEPKPAEPKS | SEQ ID no. 12 |
| TcEp13 | 9 | TSSA | TSSTPPSGTENKPATG | SEQ ID no. 94 |
| TcEp8 | 10 | TcLo 1.2 | GTSEEGSRGGSSMPS | SEQ ID no. 14 |
| TcEp9 | 11 | B13 | SPFGQAAAGDK | SEQ ID no. 15 |
| TcEp3 | 12 | TcE | KAAIAPA | SEQ ID no. 9 |
| TcEp10 | | CRA | KQRAAEATK | SEQ ID no. 16 |
| Spacer | Between | | GGASG | SEQ ID no. 99 |
| | TcEp3 and | | | |
| | 10 | | | |

After the selection of the T. cruzi polyamino acid sequences, the synthetic gene corresponding to the TxCruzi protein was produced by chemical synthesis, by the ligation gene synthesis methodology and inserted into plasmids for experimentation. The nucleotide sequence corresponding to the TxCruzi gene, containing the epitopes TcEp 1, TcEp 3, TcEp 4, TcEp 6, TcEp 8, TcEp 9, TcEp 10, TcEp 11, TcEp 12 and TcEp 13, is described in SEQ ID no. 91.

Combining the sequence of these epitopes described above with the Tx protein gave rise to the TxCruzi protein. The amino acid sequence corresponding to the TxCruzi gene, containing the epitopes TcEp 1, TcEp 3, TcEp 4, TcEp 6, TcEp 8, TcEp 9, TcEp 10, TcEp 11, TcEp 12, and TcEp 13, is described in SEQ ID NO: 90.

The synthesized gene was introduced into pET28a plasmids using the restriction sites for the enzymes Xbal and XhoI by state-of-the-art molecular biology methods. In order to identify whether the synthetic gene matched the sequence designed for the TxCruzi protein, a DH5α strain of Escherichia coli was transformed and the plasmid material analyzed by restriction enzyme digestion techniques and then sequenced, as already specified in PlatCruzi.

The analyzed plasmid harboring the correct sequence for TxCruzi was transferred to E. coli, strain BL21, in order to produce the TxCruzi protein. The BL21 strain can express T7 RNA polymerase when induced by Isopropyl β-D-1-thiogalactopyranoside (IPTG). The BL21 strain was grown overnight in LB medium at 37° C. and subsequently reseeded in the same medium with kanamycin (30 μg/ml) on a shaker at 200 rpm until an optical turbidity density of 0.6-0.8 (600 nm) was reached. Then IPTG (q.s.p. 1 mM) is added to the culture and the same culture conditions are maintained for another 3 h at 37° C.

The culture was subjected to centrifugation and the pellet resuspended in 2 mL of PBS with CelLytic (0.5×) for one hour at 4° C. After another centrifugation, the supernatant was collected and the pellet was resuspended in 8 M urea solution (pH 8.0) in the same volume as the supernatant. Equal volumes were loaded on the SDS-PAGE gel (11%, Table 5).

The TxCruzi protein was examined by SDS-PAGE to confirm its production and to determine its distribution between the soluble and insoluble fractions. As seen in FIG. 11, columns 3 and 4 (arrows) show that TxCruzi is produced as soluble and insoluble. Compared to PlatCruzi (columns 1 and 2), TxCruzi showed an improvement in the proportion of soluble protein produced, which can be attributed to the use of Tx as a receptacle protein.

### Example 19—Synthesis of SARS-CoV-2 Peptide Libraries on Cellulose Membranes and Reactivity with Sera from SARS-CoV-2 Positive and Negative Individuals

Polypeptide libraries covering all protein-coding regions of ORF3a, ORF6, ORF7, ORFS, ORF10, N, M, S, E of SARS-CoV-2 virus were synthesized based on the genomic sequence of SARS-CoV-2 isolated in Wuhan city in China and published in the GenBank database (https://www.ncbi.nlm.nih.gov/nuccore/MN908947.3?report=genbank) and were annotated as follows:

Four polypeptides not encoded by SARS-CoV-2 virus were included in the peptide library relationships to represent positive controls for the reactivity of human sera. In Table 18, A1, V5 (IHLVNNESSEVIVHK, Clostridium tetani peptide precursor), A2, V6 (GYPKDGNAFNNLDR, Clostridium tetani), A3, V7 (KEVPALTAVETGATG, human polyvirus), A4, V8 (YPYDVPDYAGYPYD, triple hemagglutinin peptide) were used as such controls. In Tables 19 and 20 A1, V4 (IHLVNNESSEVIVHK, Clostridium tetani peptide precursor), A2, V5 (GYPKDGNAFNNLDR, Clostridium tetani), A3, V6 (KEVPALTAVETGATG, human polyvirus), A4, V8 (YPYDVPDYAGYPYD, triple hemagglutinin peptide) were used as such controls.

As negative controls, the peptide-free spot reactant was used in A5, A6, K20, K21, N3, N4, O24, P1, P13, P14, Q14, Q15, R15, R16, V3, V4, V9-V24, W14 in Table 18, and A5, K19, K20, N2, N3, O23, O24, P12, P13, Q13, Q14, R15, R15, V2, V3, V17, V18 in Table 19 and Table 20.

The relationship of the synthetic linear polyamino acids is shown in Table 18, Table 19, and Table 20.

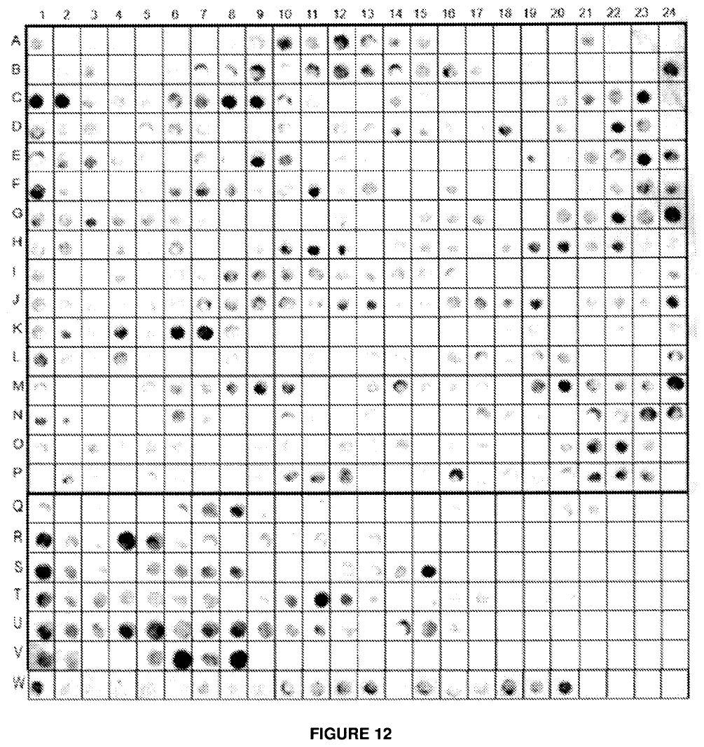

TABLE 18
List of SARS-COV-2 polyamino acid
ssynthesized for mapping IgM-reactive
epitopes from patient sera in FIG. 12.

| Spot | Polypeptides |
|---|---|
| A1 | IHLVNNESSEVIVHK |
| A2 | GYPKDGNAFNNLDRI |
| A3 | KEVPALTAVETGATN |
| A4 | [symbol] |
| A5 | |
| A6 | |
| A7 | MFVFLVLLPLVSSQC |
| A8 | VLLPLVSSQCVNLTT |
| A9 | VSSQCVNLTTRTQLP |
| A10 | VNLTTRTQLPFAVTN |
| A11 | TRQLPPAYTNSFTRG |
| A12 | FAYTNSFTRGVYYPD |
| A13 | SFTRGVYYPDKVFRS |
| A14 | VYYPDKVFRSSVLHS |
| A15 | KVFRSSVLHSTQDLF |
| A16 | SVLHSTQDLFLPFFS |
| A17 | TQDLFLFFFSNVTWF |
| A18 | LPFFSNVTWFHAIHV |
| A19 | NVTWFHAIHVSGTNG |
| A20 | HAIHVSGTNGTKRFD |
| A21 | SGTNGTKRFDNPVLP |
| A22 | TKRFDNPVLPFNDGV |
| A23 | NPVLPFNDGVYFAST |
| A24 | FNDGVYFASTEKSNI |
| B1 | YFASTEKSNIIRGWI |
| B2 | EKSNIIRGWIFGTTL |
| B3 | IRGWIFGTTLDSKTQ |
| B4 | FGTTLDSKTQSLLIV |
| B5 | DSKTQSLLIVNNATN |
| B6 | SLLIVNNATNVVIKV |
| B7 | NNATNVVIKVCEFQF |
| B8 | VVIKVCEFQFCNDPF |
| B9 | CEFQFCNDPFLGVYY |
| B10 | CNDPFLGVYYHKNNK |
| B11 | LGVVVHKNNKSWMES |
| B12 | [symbol] |
| B13 | SWMESEFRVYSSANN |
| B14 | EFRVVSSANNCTFEV |
| B15 | SSANNCTFEYVSQPF |
| B16 | CTFEYVSQPFLMDLE |
| B17 | VSQPFLMDLEGKQGN |
| B18 | LNDLEGKQGNFKNLR |
| B19 | GKQGNFKNLREFVFK |
| B20 | FKNLREFVFKNIDGY |
| B21 | EFVFKNIDGYFKIYS |
| B22 | NIDGYFKIYDKHTPI |
| B23 | FKIYSKWTPINLVRD |
| B24 | KHTPINLVRDLPQGF |
| C1 | NLVRDLPQGFSALEP |
| C2 | LPQFGSALEPLVDLP |
| C3 | SALEPLVDLPIGINI |
| C4 | LVDLPIGINITRFQT |
| C5 | IGINITRGQTLLALH |
| C6 | TRFQTLLALHRSYLT |
| C7 | LLALHRSYLTPGDSS |
| C8 | RSYLTPGDSSSGWTA |
| C9 | PGDSSSGWTAGAAAY |
| C10 | SGWTAGAAAYYVGYL |
| C11 | GAAAYYVGYLQPRTF |
| C12 | YVGYLQPRTFLLKVN |
| C13 | QPRTFLLKYNENGTI |
| C14 | LLKYNENGTITDAVD |
| C15 | ENGTITDAVDCALDP |
| C16 | TDAVDCALDPLSETK |
| C17 | CALDPLSETKCTLKS |
| C18 | LSETKCTLKSFTVEK |
| C19 | CTLKSFTVEKGIYQT |
| C20 | FTVEKGIYQTSNFRV |
| C21 | GIYQTSNFRVQPTES |
| C22 | SNFRVQPTESIVRFP |
| C23 | QPTESIVRFPNITNL |
| C24 | IVRFPNITNLCPFGE |
| D1 | NITNLCPFGEVFNAT |
| D2 | VPFGEVFNATRFASV |
| D3 | VFNATRFASVYAWNR |
| D4 | RFASVYAWNRKRISN |
| D5 | YAWNRKRISNCVADY |
| D6 | KRISNCVADYSVLYN |
| D7 | CVADYSVLYNSASFS |
| D8 | SVLVNSASFSTFKCV |
| D9 | SASFSTFKCYGVSPT |
| D10 | TFKCYGVSPTKLNDL |
| D11 | GVSPTKLNDLVFTNV |
| D12 | KLNDLCFTNVYADSF |
| D13 | CFTNVYADSFVIRGD |
| D14 | YASSFVIRGDEVRQI |
| D15 | VIRGDEVRQIAPGQT |
| D16 | EVRQIAPGQTGKIAD |
| D17 | APGQTGKIADYNYKL |
| D18 | GKIADVNYKLPDDFT |
| D19 | VNYELPDDFTGCVIA |
| D20 | PDDFTGCVIAWNSNN |
| D21 | GCVIAWNSNNLDKKV |
| D22 | WNSNNLDSKVGGNYN |
| D23 | LDSKVGGNYNYLYRL |
| D24 | GGNVNYLYRLFRKSN |
| E1 | YLYRLFRKSNLKPFE |
| E2 | FRKSNLKPFERDIST |
| E3 | LKPFERDISTEIVQA |
| E4 | RDISTEIVQAGSTPC |
| E5 | EIYQAGSTPCNGVEG |
| E6 | GSTPCNGVEGFNCVF |
| E7 | NGVEGFNCYFPLQSY |
| E8 | FNCYFPLQSYGFQPT |
| E9 | PLQSYGFQPTNGVGY |
| E10 | GFQPTNGVGYQPYRV |
| E11 | NGVGYQPYRVVVLSF |
| E12 | QPVRVVVLSFELLHA |
| E13 | VVLSFELLRAPATVC |
| E14 | ELLHAPATYCGPKKS |
| E15 | PATVCGPKKSTNLVK |
| E16 | GPKKSTNLVKNKCVN |
| E17 | TNLVKNKCVNFNFNG |
| E18 | NKCVNFNFNGLTGTG |
| E19 | FNFNGLTGTGVLTES |
| E20 | LTGTGVLTESNKKFL |
| E21 | VLTESNKKFLPFQQF |
| E22 | NKKFLPFQQFGRDIA |
| E23 | PFQQFGRDIADTTDA |
| E24 | GRDIADTTDAVRDPQ |
| F1 | DTTDAVRDPQTLEIL |
| F2 | VRDPQTLEILDITPC |
| F3 | TLEILDTTPCSFGGV |
| F4 | DITPCSFGGVSVITP |
| F5 | SFGGVSVITPGTNTS |
| F6 | SVITPGTNTSNQVAV |
| F7 | GTNTSNQVAVLYQDV |
| F8 | NQVAVLYQDVNCTEV |
| F9 | LYQDVNCTEVPVAIH |
| F10 | NCTEVPVAIHADQLT |
| F11 | PVAIHADQLTPTWRV |
| F12 | ADQLTPTWRVYSTGS |
| F13 | PTWRVYSTGSNVFQT |
| F14 | YSTGSNVFQTRAGCL |
| F15 | NVFQTRAGCLIGAEH |
| F16 | RAGCLIGAEHVNNSY |
| F17 | IGAEHVNNSYECDIP |
| F18 | VNNSYECDIPIGAGI |
| F19 | ECDIPIGAGICASYQ |
| F20 | IGAGICASYQTQTNS |
| F21 | CASYQTQTNSPRRAR |
| F22 | TQTNSPRRARSVASQ |
| F23 | PRRARSVASQSIIAY |
| F24 | SVASQSIIAYTMSLG |
| G1 | SIIAYTMSLGAENSV |
| G2 | TMSLGAENSVAYSNN |
| G3 | AENSVAYSNNSIAIP |
| G4 | AVSNNSIAIPTNFTI |
| G5 | SIAIPTNFTISVTTE |
| G6 | TNFTISVTTEILPVS |
| G7 | SVTTEILPVSMTNTS |
| G8 | ILPVSMTKTSVDCTM |
| G9 | MTKTSVDCTMYICGD |
| G10 | VDCTMYICGDSTECS |
| G11 | YICGDSTECSNLLLQ |
| G12 | STECSNLLLQYGSFC |
| G13 | NLLLQYGSFCTQLNR |
| G14 | YGSFCTQLNRALTGI |
| G15 | TQLNRALTGIAVEQD |
| G16 | ALTGIAVEQDKNTQE |
| G17 | AVEQDKNTQEVFAQV |
| G18 | KNTQEVFAQVKQIYK |
| G19 | VFAQVKQIYKTPPIN |
| G20 | KQIVKTPPIKDFGGF |
| G21 | TPPIKDFGGFNFSQI |
| G22 | DFGGFNFSQILPDPS |
| G23 | NFSQILPDPSKPSKR |
| G24 | LPDPSKPSKRSFIED |
| H1 | KPSKRSFIEDLLFNK |
| H2 | SFIEDLLFNKVTLAD |
| H3 | LLFNKVTLADAGFIK |
| H4 | VTLADAGFIKQYGDC |
| H5 | AGFIKQYGDCLGDIA |
| H6 | QYGDCLGDIAARDLI |
| H7 | LGDIAARDLICAQKF |
| H8 | ARDLICAQKFNGLTV |
| H9 | CAQKFNGLTVLPPLL |
| H10 | NGLTVLPPLLTDEMI |
| H11 | LPPLLTDEMIAQYTS |
| H12 | TDEMIAQYTSALLAG |
| H13 | AQYTSALLAGTITSG |
| H14 | ALLAGTITSGWTFGA |
| H15 | TITSGWTFGAGAALQ |
| H16 | WTFGAGAALQIPFAM |
| H17 | GAALQIPFAMQMAYR |
| H18 | IPFAMGMAYRFNGIG |
| H19 | QMAYRFNGIGVTQNV |
| H20 | FNGIGVFQNVLYENQ |
| H21 | VTQNVLYENQKLIAN |
| H22 | LYENQKLIANQFNSA |
| H23 | KLIANQFNSAIGKIQ |
| H24 | QGNSAIGMIQDSLSS |
| I1 | IGKIQDSLSSTASAL |
| I2 | DSLSSTASALGKLQD |
| I3 | TASALGKLQDVVNQN |
| I4 | GKLQDVVNQNAQALN |
| I5 | VVNQNAQALNTLVKQ |
| I6 | AQALNTLVKQLSSNF |
| I7 | TLVKQLSSNFGAISS |
| I8 | LSSNFGAISSVLNDI |
| I9 | GAISSVLNDILSRLD |
| I10 | VLNDILSRLDKVEAE |
| I11 | LSRLDKVEAEVQIDR |
| I12 | KVEAEVQIDRLITGR |
| I13 | VQIDRLITGRLQSLQ |
| I14 | LITGRLQSLQTYVTQ |
| I15 | LQSLQTYVTQQLIRA |
| I16 | TYVTQQLIRAAEIRA |
| I17 | QLIRAAEIRASANLA |
| I18 | AEIRASANLAATKMS |
| I19 | SANLAATKMSECVLG |
| I20 | ATKMSECVLGQSKRV |
| I21 | ECVLGQSKRVDFCGK |
| I22 | QSKRVDFCGKGYHLM |
| I23 | DFCGKGYHLMSFPQS |
| I24 | GYHLMSFPQSAPHGV |
| J1 | SFPQSAPHGVVFLHV |
| J2 | APHGVVFLHVTVVPA |
| J3 | VFLHVTYVPAQEKNF |
| J4 | TYVPAQEKNFTTAPA |
| J5 | QEKNFTTAPAICHDG |
| J6 | TTAPAICHDGKAHFP |
| J7 | ICHDGKAHFPREGVF |
| J8 | KAHFPREGVFVSNGT |
| J9 | REGVFVSNGTHWFVT |
| J10 | VSNGTHWFVTQRNFY |
| J11 | HWFVTQRNFVEPQII |
| J12 | QRNFYEPQIITTDNT |
| J13 | EPQIITTDNTFVSGN |
| J14 | TTDNTFVSGNCDVVI |
| J15 | FVSGNCDVVIGIVNN |
| J16 | CDVVIGIVNNTVYDP |
| J17 | GIVNNTVYDPLQPEL |
| J18 | TVYDPLQPELDSFKE |
| J19 | LQPELDSFKEELDKY |
| J20 | DSFKEELDKYFKNHT |
| J21 | ELDKYFKNHTSPDVD |
| J22 | FKNHTSPVDVLGDIS |
| J23 | SPDVDLGDISGINAS |
| J24 | LGDISGINASVVNIQ |
| K1 | GINASVVNIQKEIDR |
| K2 | VVNIQKEIDRLNEVA |
| K3 | KEIDRLNEVAKNLNE |
| K4 | LNEVAKNLNESLIDL |
| K5 | KNLNESLTDLQELGK |
| K6 | SLIDLQELGKYEQVI |
| K7 | QELGKYEQYIKWPWY |
| K8 | [symbol] |
| K9 | KWPWYIWLGFIAGLI |
| K10 | IWLGFIAGLIAIVMV |
| K11 | IAGLIAIVMVTIMLC |
| K12 | AIVMVTIMLCCMTSC |
| K13 | TIMLCCMTSCCSLCK |
| K14 | CMTSCCSCLKGCCSC |
| K15 | CSCLKGCCSCGSCCK |
| K16 | GCCSCGSCCKFDEDD |
| K17 | GSCCKFDEDDSEPVL |
| K18 | FDEDDSEPVLKGVKL |
| K19 | DDSEPVLKGVKLHYT |
| K20 | |
| K21 | |
| K22 | MDLFMRIFTIGTVTL |
| K23 | RIFTIGTVTLKQGEI |
| K24 | GTVTLKQGEIKDATP |
| L1 | KQGEIKDATPSDFVR |
| L2 | KDATPSDFVRATATI |
| L3 | SDFVRATATIPIQAS |
| L4 | ATATIPIQASLPFGW |
| L5 | PIQASLPFGWLIVGV |
| L6 | LPFGWLIVGVALLAV |
| L7 | LIVGVALLAVFQSAS |
| L8 | ALLAVFQSASKIITL |
| L9 | FQSASKIITLKKRWQ |
| L10 | KIITLKKRWQLALSK |
| L11 | KKRWQLALSKGVHFV |
| L12 | LALSKGVHFVCNLLL |
| L13 | GVHFVCNLLLLFVTV |
| L14 | CNLLLLFVTVYSHLL |
| L15 | LFVTVYSHLLLVAAG |
| L16 | YSHLLLVAAGLEAPF |
| L17 | LVAAGLEAPFLYLYA |
| L18 | LEAPFLYLYALVYFL |
| L19 | LYLYALVYFLQSINF |
| L20 | LVYFLQSINFVRIIM |
| L21 | QSINFVRIIMRLWLC |
| L22 | VRIIMRLWLCWKCRS |
| L23 | RLSLCWKCRSKNPLL |
| L24 | WKCRSKNPLLYDANY |
| M1 | KNPLLYDANYFCLWH |
| M2 | YDANYFLCWHTNCVD |
| M3 | FLCWHTNCYDYCIPY |
| M4 | TNCYDYCIPYNSVTS |
| M5 | VCIPYNSVTSSIVIT |
| M6 | NSVTSSIVITSGDGT |
| M7 | SIVITSGDGTTSPIS |
| M8 | SGDGTTSPISEHDVQ |
| M9 | TSPISEHDYQIGGVT |
| M10 | EHDYQIGGYTEKWES |
| M11 | IGGYTEKWESGVKDS |
| M12 | EKWESGVKDCVVLHS |
| M13 | GVKDCVVLHSYFTSD |
| M14 | VVLHSYFTSDYYQLY |
| M15 | YFTSDYYQLYSTQLS |
| M16 | YYQLYSTQLSTDTGV |
| M17 | STQLSTDTGVEHVTF |
| M18 | TDTGVEHVTFFIYNK |
| M19 | EHVTFFIYNKIVDEP |
| M20 | FIYNKIVDEPEEHYQ |
| M21 | EEHVQIHTIDGSSGV |
| M22 | IHTIDGSSGVVNPVM |
| M23 | GSSGVVNPVMEPIYD |
| M24 | VNPVMEPIYDEPTTT |
| N1 | EPIYDEPTTTTSVPL |
| N2 | |
| N3 | |
| N4 | |
| N5 | MADSNGTITVEELKK |
| N6 | GTITVEELKKLLEQW |
| N7 | EELKKLLEQWNLVIG |
| N8 | LLEQWNLVIGFLFLT |
| N9 | NLVIGFLFLTWICLL |
| N10 | FLFLTWICLLQFAYA |
| N11 | WICLLQFAYANRNRF |
| N12 | QFAYANRNRFLVIIK |
| N13 | NRNRFLVIIKLIFLW |
| N14 | LYIIKLTFLWLLWPV |
| N15 | LIFLWLLWPVTLACF |
| N16 | LLWPVTLACFVLAAV |
| N17 | TLACFVLAAVYRINW |
| N18 | VLAAVYRINWITGGI |
| N19 | YRINWITGGIAIAMA |
| N20 | ITGGIAIAMACLVGL |
| N21 | AIAMACLVGLMWLSY |
| N22 | CLVGLMWLSYFIASF |
| N23 | MWLSYFIASFRLFAR |
| N24 | FIASFRLFARTRWMW |
| O1 | RLFARTRSMWSFNPE |
| O2 | TRSMWSFNPETNILL |
| O3 | [symbol] |
| O4 | TNILLNVPLHGTILT |
| O5 | NVPLHGTILTRPLLE |
| O6 | GTILTRPLLESELVI |
| O7 | RPLLESELVIGAVIL |
| O8 | SELVIGAVILRGHLR |
| O9 | GAVILRGHLRIAGHH |
| O10 | RGHLRIAGHHLGRCD |
| O11 | IAGHHLGRCDIKDLP |
| O12 | LGRCDIKDLPKEITV |
| O13 | IKDLPKEITVATSRT |
| O14 | KEITVATSRTLSYYK |
| O15 | ATSRTLSYYKLGASQ |
| O16 | LSYVKLGASQRVAGD |
| O17 | LGASQRVAGDSGFAA |
| O18 | RVAGDSGFAAYSRYR |
| O19 | SGFAAYSRYRIGNYK |
| O20 | YSRYRIGNYKLNTDH |
| O21 | IGNYKLNTDHSSSSD |
| O22 | LNTDHSSSSDNIALL |
| O23 | TDHSSSSDNIALLVQ |
| O24 | |
| P1 | |
| P2 | MFHLVDFQVTIAEIL |
| P3 | DFQVTIAEILLIIMR |
| P4 | IAEILLIIMRTFKVS |
| P5 | LIIMRTFKVSIWNLD |
| P6 | TFKVSIWNLDYIINL |
| P7 | IWNLDYIINLIIKNL |
| P8 | YIINLIIKNLSKSLT |
| P9 | IIKNLSKSLTENKYS |
| P10 | SKSLTENKYSQLDEE |
| P11 | ENKYSQLDEEQPMEI |
| P12 | NKYSQLDEEQPMEID |
| P13 | |
| P14 | |
| P15 | MKIILFLALITLATC |
| P16 | FLALITLATCELVHY |
| P17 | TLATCELYHYQECVR |
| P18 | ELYHVDECVRGTTVL |
| P19 | QECVRGTTVLLKEPC |
| P20 | GTTVLLKEPCSSGTY |
| P21 | LKEPCSSGTYEGNSP |
| P22 | SSGTVEGNSPFHPLA |
| P23 | EGNSPFHLPADNKFA |
| P24 | FHPLADNKFALTCFS |
| Q 1 | DNKFALTCFSTQFAF |
| Q 2 | LTCFSTQFAFACPDG |
| Q 3 | TQFAFACPDGVKHVY |
| Q 4 | ACPDGVKHVYQLRAR |
| Q 5 | VKHVYQLRARSVSPK |
| Q 6 | QLRARSVSPKLFIRQ |
| Q 7 | SVSPKLFIRQEEVQE |
| Q 8 | LFIRQEEVQELYSPI |
| Q 9 | EEVQELYSPIFLIVA |
| Q10 | LYSPIFLIVAAIVFI |
| Q11 | FLIVAAIVFITLCFT |
| Q12 | AIVFITLCFTLKRKT |
| Q13 | IVFITLCFTLKRKTE |
| Q14 | |
| Q15 | |
| Q16 | MKFLVFLGIITTVAA |
| Q17 | FLGIITTVAAFHQEC |
| Q18 | TTVAAFHQECSLQSC |
| Q19 | FHQECSLQSCTQHQP |
| Q20 | SLQSCTQHQPYVVDD |
| Q21 | TQHQPYVVDDPCPIH |
| Q22 | YVVDDPCPIHFYSKW |
| Q23 | PCPIHFYSKWYIRVG |
| Q24 | FYSKWYIRVGARKSA |
| R 1 | YIRVGARKSAPLIEL |
| R 2 | ARKSAPLEILCVDEA |
| R 3 | PLIELCVDEAGSKSP |
| R 4 | CVDEAGSKSPIQVID |
| R 5 | GSKSPIQYIDIGNYT |
| R 6 | IQYIDIGNYTVSCLP |
| R 7 | IGNYTVSCLPFTINC |
| R 8 | VSCLPFTINCQEPKL |
| R 9 | FTINCQEPKLGSLVV |
| R10 | QEPKLGSLVVRCSFY |
| R11 | GSLVVRCSFYEDFLE |
| R12 | RCSFYEDFLEYHDVR |
| R13 | EDFLEYHDVRVVLDF |
| R14 | DFLEYHDVRVVLDFI |
| R15 | |
| R16 | |
| R17 | MSDNGPQNQRNAPRI |
| R18 | PQNQRNAPRITFGGP |
| R19 | NAPRITFGGPSDSTG |
| R20 | TFGGPSDSTGSNQNG |
| R21 | SDSTGSNQNGERSGA |
| R22 | SNQNGERSGARSKQR |
| R23 | ERSGARSKQRRPQGL |
| R24 | RSKQRRPQGLPNNTA |
| S 1 | RPQGLPNNTASWFTA |
| S 2 | PNNTASWFTALTQHG |
| S 3 | SWFTALTQHGKEDLK |
| S 4 | LTQHGKEDLKFPRGQ |
| S 5 | KEDLKFPRGQGVPIN |
| S 6 | FPRGQGVPINTNSSP |
| S 7 | GYPINTNSSPDDQIG |
| S 8 | TNSSPDDQIGYYRRA |
| S 9 | DDQIGVYRRATRRIR |
| S10 | YYRRATRRIRGGDGK |
| S11 | TRRIRGGDGKMKDLS |
| S12 | GGDGKMKDLSPRWYF |
| S13 | MKDLSPRWYFYYLGT |
| S14 | PRWYFYYLGTGPEAG |
| S15 | YYLGTGPEAGLPYGA |
| S16 | GPEAGLPYGANKDGI |
| S17 | LPYGANKDGIIWVAT |
| S18 | NKDGIIWVATEGALN |
| S19 | IWVATEGALNTPKDH |
| S20 | EGALNTPKDHIGTRN |
| S21 | TPKDHIGTRNPANNA |
| S22 | IGTRNPANNAAIVLQ |
| S23 | PANNAAIVLQLPQGT |
| S24 | AIVLQLPQGTTLPKG |
| T 1 | LPQGTTLPKGFYAEG |
| T 2 | TLPKGFYAEGSRGGS |
| T 3 | FYAEGSRGGSQASSR |
| T 4 | SRGGSQASSRSSSRS |
| T 5 | QASSRSSSRSRNSSR |
| T 6 | SSSRSRNSSRNSTPG |
| T 7 | RNSSRNSTPGSSRGT |
| T 8 | NSTPGSSRGTSPARM |
| T 9 | SSRGTSPARMAGNGG |
| T10 | SPARMAGNGGDAALA |
| T11 | AGNGGDAALALLLLD |
| T12 | DAALALLLLDRLNQL |
| T13 | LLLLDRLNQLESKMS |
| T14 | RLNQLESKMSGKGQQ |
| T15 | ESKMSGKGQQQQGQT |
| T16 | GKGQQQQGQTVTKKS |
| T17 | QQGQTVTKKSAAEAS |
| T18 | VTKKSAAEASKKPRQ |
| T19 | AAEASKKPRQKRTAT |
| T20 | KKPRQKRTATKAYVN |
| T21 | KRTATKAVNVTQAFG |
| T22 | KAYNVTQAFGRRGPE |
| T23 | TQAFGRRGPEQTQGN |
| T24 | RRGPEQTQGNFGDQE |
| U 1 | QTQGNFGDQELIRQG |
| U 2 | FGDQELIRQGTDYKH |
| U 3 | LIRQGTDYKHWPQIA |
| U 4 | TDYKHWPQIAQFAPS |
| U 5 | WPQIADFAFSASAFF |
| U 6 | QFAPSASAFFGMSRI |
| U 7 | ASAFFGMSRIGMEVT |
| U 8 | GMSRIGMEVTPSGTW |
| U 9 | GMEVTPSGTWLTYTG |
| U10 | PSGTWLTYTGAIKLD |
| U11 | LTYTGAIKLDDKDPN |
| U12 | AIKLDDKDPNFKDQV |
| U13 | DKDPNFKDQVILLNK |
| U14 | FKDQVILLNKHIDAY |
| U15 | ILLNKHIDAYKTFPP |
| U16 | HIDAYKTFPPTEPKK |
| U17 | KTFPPTEPKKDKKKK |
| U18 | TEPKKDKKKKADETQ |
| U19 | DKKKKADETQALPQR |
| U20 | ADETQALPQRQKKQQ |
| U21 | ALPQRQKKQQTYTLL |
| U22 | QKKQQTVTLLPAADL |
| U23 | TVTLLPAADLDDFSK |
| U24 | PAADLDDFSKQLQQS |
| V 1 | DDFSKQLQQSMSSAD |
| V 2 | KQLQQSMSSADSTQA |
| V 3 | |
| V 4 | |
| V 5 | IHLVNNESSEVIVHK |
| V 6 | GYPKDGNAFNNLDRI |
| V 7 | KEVPALTAVETGATN |
| V 8 | YPVDVPSYAGYPYDV |
| V 9 | |
| V 10 | |
| V 11 | |
| V 12 | |
| V 13 | |
| V 14 | |
| V 15 | |
| V 16 | |
| V 17 | |
| V 18 | |
| V 19 | |
| V 20 | |
| V 21 | |
| V 22 | |
| V 23 | |
| V 24 | |
| W1 | MVSFVSEETGTLIVN |
| W2 | SEETGTLIVNSVLLF |
| W3 | TLIVNSVLLFLAFVV |
| W4 | SVLLFLAFVVFLLVT |
| W5 | LAFVVFLLVTLAILT |
| W6 | FLLVTLATLTALRLC |
| W7 | LAILTALRLCAYCCN |
| W8 | ALRLCAYCCNIVNVS |
| W9 | AYCCNIVNVSLVKPS |
| W10 | IVNVSLVKPSFYVYS |
| W11 | LVKPSFYVYSRVKNL |
| W12 | FYVYSRVKNLNSSRV |
| W13 | RVKNLNSSRVPDLLV |
| W14 | |
| W15 | MGYINVFAFPFTIYS |
| W16 | VFAFPFTIYSLLLCR |
| W17 | FTIYSLLLCRMNSRN |
| W18 | LLLCRMNSRNYIAQV |
| W19 | MNSRNVIAQVDVVNF |
| W20 | RNYIAQVDVVNFNLT |

[symbol]

indicates data missing or illegible when filed

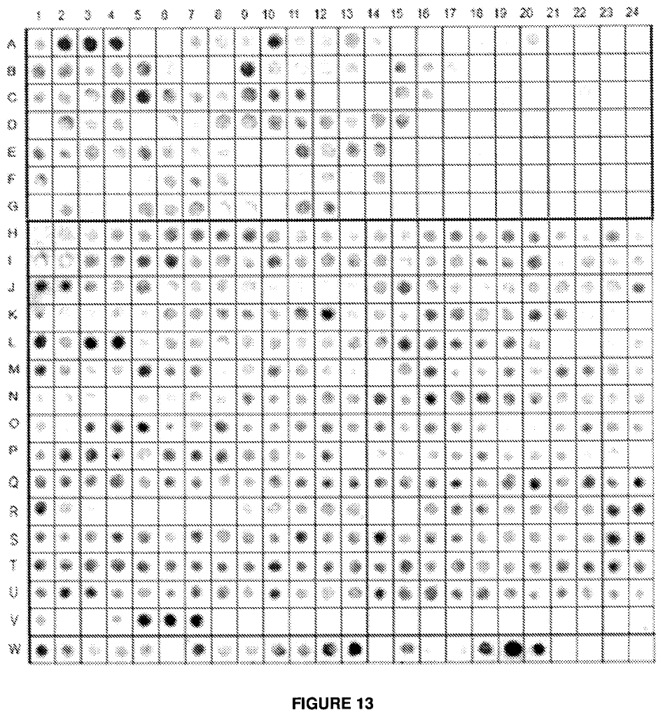

TABLE 19
List of SARS-COV-2 polyamino acids
synthesized for mapping IgG-reactive
epitopes from patient sera in FIG. 13.

| Spot | Polypeptide |
|---|---|
| A1 | IHLVNNESSEVIVHK |
| A2 | GVPKDGNAFNNLDKI |
| A3 | KEVPALTAVETGATN |
| A4 | VPVDVPDVAGVPVDV |
| A5 | |
| A6 | MFVFLVLLPLVSSQC |
| A7 | VLLPLVSSQCVNLTT |
| A8 | VSSQCVNLTTKTQLP |
| A9 | VNLTTRTQLPPAYTN |
| A10 | KTQLPPAVTNSFTNG |
| A11 | SFTNGVYVPDKVFKS |
| A12 | VYYPDKVFRSSVLHS |
| A13 | KVFRSSVLHSTQDLF |
| A14 | KVFRSSVLHSTQDLF |
| A15 | SVLHSTQDLFLPFFS |
| A16 | TQDLFLPFFSNVTWF |
| A17 | LPFFSNVTWFHAIHV |
| A18 | NVTWFHAIHVSGTNG |
| A19 | HAIHVSGTNGTKRFD |
| A20 | SGTNGTKRFDNPVLP |
| A21 | TKRFDNPVLPFNDGV |
| A22 | NPVLPFNDGVYFAST |
| A23 | FNDGVVFASTEKSNI |
| A24 | YFASTEKSNIIRGWI |
| B1 | EKSNIIRGWIFGTTL |
| B2 | IRGWIFGTTLDSKTQ |
| B3 | FGTTLDSKTQSLLIV |
| B4 | DSKTQSLLTVNNATN |
| B5 | SLLIVNNATNVVIKV |
| B6 | NNATNVVIKVDEFQF |
| B7 | VVIKVCEFQFCNDPF |
| B8 | CEFQFCNDPFLGVYY |
| B9 | CNDPFLGVYVHKNNK |
| B10 | [symbol] |
| B11 | [symbol] |
| B12 | SWMESEFRVYSSANN |
| B13 | EFRVYSSANNCTFEV |
| B14 | SSANNCTFEYVSQPF |
| B15 | [symbol] |
| B16 | VSQPFLMDLEGKQGN |
| B17 | LMDLEGKQGNFKNLK |
| B18 | GKQGNFKKLREFVFK |
| B19 | FKNLREFVFKNIDGY |
| B20 | EFVFKNIDGYFKIYS |
| B21 | NIDGYFKIYSKHTPI |
| B22 | FKIVSKHTPINLVRD |
| B23 | KHTPINLVRDLPQGF |
| B24 | NLVRDLPQGFSALEP |
| C1 | LPQGFSALEPLVDLP |
| C2 | SALEPLVDLPIGINI |
| C3 | LVDLPIGINITRGQT |
| C4 | IGINITHFQTLLALH |
| C5 | TRFQTLLALHRSYLT |
| C6 | LLALHRSYLTPGDSS |
| C7 | RSYLTPGDSSSGWTA |
| C8 | PGDSSSGWTAGAAAY |
| C9 | SGWTAGAAAVVVGVL |
| C10 | GAAAYYCGYLQPRTF |
| C11 | YVGVLQPRTFLLKVN |
| C12 | QPRTFLLKYNENGTI |
| C13 | LLKYNENGTITDAVD |
| C14 | ENGTITDAVDCALDP |
| C15 | TDAVDCALDPLSETK |
| C16 | CALDPLSETKCTLKS |
| C17 | LSETKCTLKSFTVEK |
| C18 | CTLKSFTVEKGIYQT |
| C19 | FTVEKGIVQTSNFRV |
| C20 | GIYQTSNFRVQPTES |
| C21 | SNFRVQPTESIVRFP |
| C22 | QPTESIVRFPNTTNL |
| C23 | IVRFPNITNLCPFGE |
| C24 | NITNLCPFGEVFNAT |
| D1 | CPFGEVFNATRFASV |
| D2 | VFNATRFASVYAWNR |
| D3 | FRASVYAWNKKKISN |
| D4 | YAWNRKRESNCVADY |
| D5 | KRISNCVADYSVLYN |
| D6 | CVADYSVLYNSASFS |
| D7 | SVLYNSASFSTFKCV |
| D8 | SASFSTFNCYGVSPT |
| D9 | YFKCYGVSPTKLNDL |
| D10 | GVSPTKLNDLCFTNV |
| D11 | KLNDLVFTNVYADSF |
| D12 | CFTNVYADSFVIRGD |
| D13 | YADSFVIRGDEVRQI |
| D14 | VIRGDEVRQIAPGQT |
| D15 | EVRQIAPGQTGKIAD |
| D16 | APGQTGKIADYNYKL |
| D17 | GKIAKVNVKLPDDFT |
| D18 | YNYKLPDDFTGCVIA |
| D19 | PDDFTGCVIAWNSNN |
| D20 | GCVIAWNSNNLDSKV |
| D21 | WNSNNLDSKVGGNYN |
| D22 | LDSKVGGNYNYLTRL |
| D23 | GGNYNYLYNLFRKSN |
| D24 | YLYRLFRKSNLKPFE |
| E1 | FRKSNLKPFERDIST |
| E2 | LKPFERDISTEIYQA |
| E3 | RDISTEIYQAGSTPC |
| E4 | EIYQAGSTPCNGVEG |
| E5 | GSTPCNGVEGFNCYF |
| E6 | NGVEGFNCYFPLQSY |
| E7 | FNCYFPLQSYGFQPT |
| E8 | PLQSYGFQPTNGVGY |
| E9 | GFQPTNGVGYQPYRV |
| E10 | NGVGYQPYRVVVLSF |
| E11 | QPYRVVVLSFELLHA |
| E12 | VVLSFELLHAPATVC |
| E13 | ELLRAPATVCGPKKS |
| E14 | PATVCGPKKSTNLVK |
| E15 | GPKKSTNLVKKKCVN |
| E16 | TNLVKKKCVNFNFNG |
| E17 | NKCVNFNFNGLTGTG |
| E18 | FNFNGLTGTGVLTES |
| E19 | LTGTGVLTESNKKFL |
| E20 | VLTESNKKFLPFQQF |
| E21 | NKKFLPFQQFGRDIA |
| E22 | PFQQFGRDIADTTDA |
| E23 | GRDIADTTDAVRDPQ |
| E24 | DTTDAVRDPQTLEKL |
| F1 | VRDPQTLEILDITPC |
| F2 | TLEILDITPCSFGGV |
| F3 | DITPCSFGGVSVITP |
| F4 | SFGGVSVITPGTNTS |
| F5 | SVITPGTNTSNQVAV |
| F6 | GTNTSNQVAVLVQDV |
| F7 | NQVAVLYQDVNCTEV |
| F8 | LYQDVNCTEVPVAIH |
| F9 | NCTEVPVAIHADQLT |
| F10 | PVAIHADQLTPTWRV |
| F11 | ADQLTPTWRVYSTGS |
| F12 | PTWRVYSTGSNVFQT |
| F13 | YSTGSNVFQTRAGCL |
| F14 | NVFQTRAGCLIGAEH |
| F15 | RAGCLIGAEHVNNSY |
| F16 | IGAEHVNNSYECDIP |
| F17 | VNNSYECDIPIGAGI |
| F18 | ECDIPIGAGICASVQ |
| F19 | IGAGICASVQTQTNS |
| F20 | CASVQTQTNSPRRAR |
| F21 | TQTNSPRRARSVASQ |
| F22 | PRRARSVASQSIIAY |
| F23 | SVASQSIIAYTMSLG |
| F24 | SIIAYTMSLGAENSV |
| G1 | TNSLGAENSVAYSNN |
| G2 | AENSVAYSNNSIAIP |
| G3 | AYSNNSIAIPTNFTI |
| G4 | STAIFTNFTISVTTE |
| G5 | TNFTTSVTTEILPVS |
| G6 | SVTTEILPVSMTNTS |
| G7 | ILPVSNTKTSVDCTM |
| G8 | MTKTSVDCTMYICGD |
| G9 | VDCTMYICGDSTECS |
| G10 | VICGDSTECSNLLLQ |
| G11 | STECSNLLLQYGSFC |
| G12 | NLLLQYGSFCTQLNR |
| G13 | YGSFCTQLNRALTGI |
| G14 | TQLNRALTGIAVEQD |
| G15 | [symbol] |
| G16 | AVEQDKNTQEVFAQV |
| G17 | KNTQEVFAQVKQIVK |
| G18 | VFAQVKQIYETPPIK |
| G19 | KQIYKTPPIKDFGGF |
| G20 | TPPIKDFGGFNFSQI |
| G21 | DFGGFNFSQILPDPS |
| G22 | NFSQILPDPSKPSKR |
| G23 | LPDFSKPSKRSFIED |
| G24 | KPSKRSFIEDLLFNK |
| H1 | SFIEDLLFNKVTLAD |
| H2 | LLFNKVTLADAGFIK |
| H3 | VTLADAGFIKQYGDS |
| H4 | AGFIKQYGDCLGDIA |
| H5 | QYGDCLGDIAARDLI |
| H6 | LGDIAARDLICAQKF |
| H7 | ARDLICAQKFNGLTV |
| H8 | CAQKFNGLTVLPPLL |
| H9 | NGLTVLPPLLTDEMI |
| H10 | LPPLLTDEMIAQYTS |
| H11 | TDEMIAQVTSALLAG |
| H12 | AQYTSALLAGTITSG |
| H13 | ALLAGTITSGWTFGA |
| H14 | YITSGWTFGAGAALQ |
| H15 | WTFGAGAALQIPFAM |
| H16 | GAALQIPFAMQMAYR |
| H17 | IPFAMQMAYRFNGIG |
| H18 | QMAYRNFIGIVTQNV |
| H19 | FNGIGVTQNVLYENQ |
| H20 | YTQNVLYENQKLIAN |
| H21 | LYENQKLIANQFNSA |
| H22 | KLIANQFNSAIGKIQ |
| H23 | QFNSIAGKIQDSLSS |
| H24 | IGKIQDSLSSTASAL |
| I1 | DSLSSTASALGKLQD |
| I2 | TASALGKLQDVVNQN |
| I3 | GKLQDVVNQNAQALN |
| I4 | VVNQMAQALNTLVKQ |
| I5 | AQALNTLVKQLSSNF |
| I6 | TLVKQLSSNFGAISS |
| I7 | LSSNFGAISSVLNDI |
| I8 | GAISSVLNDILSRLD |
| I9 | VLNDILSRLDNVEAE |
| I10 | LSRLDRVEAEVQIDR |
| I11 | KVEAEVQIDRLITGS |
| I12 | VQIDRLITGRLQSLQ |
| I13 | LITGRLQSLQTYVTQ |
| I14 | LQSLQTYVTQQLIRA |
| I15 | TYVTQQLIRAAEIRA |
| I16 | QLTRAAEIRASANLA |
| I17 | AETRASANLAATKMS |
| I18 | SANLAATKMSECVLG |
| I19 | ATKMSECVLGQSKKV |
| I20 | ECVLGQSKRVDFCGK |
| I21 | QSKRVDFCGKGYHLM |
| I22 | DFCGKGYHLMSFPQS |
| I23 | GYHLMSFPQSAPHGV |
| I24 | SFPQSAPHGVVFLHV |
| J1 | APHGVVFLHVTVVPA |
| J2 | VFLHVTVVPAQEKNF |
| J3 | TYVPAQEKNFTTAPA |
| J4 | [symbol] |
| J5 | TTAPAICMDGKAMFP |
| J6 | ICHDGKAMFPREGVF |
| J7 | KAHFPREGVFVSNGT |
| J8 | REGVFVSNGTHWFVT |
| J9 | VSNGTHWFVTQRNFY |
| J10 | HWFVTQRNFYEPQII |
| J11 | QRNFYEPQIITTDNT |
| J12 | EPQIITTDNTFVSGN |
| J13 | TTDNTFYSGNCDVVI |
| J14 | FVSGNCDVVIGIVNN |
| J15 | CDVVIGIVNNTVYDP |
| J16 | GIVNNTVYDPLQPEL |
| J17 | TVYDPLQPELDSFKE |
| J18 | LQPELDSFKEELDKY |
| J19 | DSFKEELDKYFKNHT |
| J20 | ELDKYFKNHTSPDVD |
| J21 | FKNHTSPDVDLGDIS |
| J22 | SPDVDLGDISGINAS |
| J23 | LGDISGINASVVNIQ |
| J24 | GINASVVNIQKEIDR |
| K1 | VVNIQKEIDRLNEVA |
| K2 | KEIDRLNEVAKNLNE |
| K3 | LNEVAKNLNESLIDL |
| K4 | KNLNESLIDLQELGK |
| K5 | SLIDLQELGKYEQYI |
| K6 | QELGKYEQYIKWPWY |
| K7 | YEQVIKWPWYDWLGF |
| K8 | KWPWYIWLGFIAGLI |
| K9 | IWLGFIAGLIAIVMV |
| K10 | IAGLIAIVMVTIMLC |
| K11 | ATVMVTIMLCCMTSC |
| K12 | TIMLCCMTSCCSCLM |
| K13 | CMTSCCSCLKGCCSC |
| K14 | CSCLKGCCSCGSCCK |
| K15 | GCCSCGSCCKFDEDD |
| K16 | GSCCKFDEDDSEPVL |
| K17 | FDEDDSEPVLKGVKL |
| K18 | DDSEPVLKGVKLHVT |
| K19 | |
| K20 | |
| K21 | MDLFMRIFTIGTVTL |
| K22 | RIFTIGTVTLKQGEI |
| K23 | GTVTLKQGEIKDATP |
| K24 | KQGEIKDATPSDFVR |
| L1 | KDATPSDFVRATATI |
| L2 | SDFVRATATIPTQAS |
| L3 | ATATIPIQASLPFGW |
| L4 | PIQASLPFGWLIVGV |
| L5 | LPFGWLIVGVALLAV |
| L6 | LIVGVALLAVFQSAS |
| L7 | ALLAVFQSASKITTL |
| L8 | FQSASKIITLKKRWQ |
| L9 | KIITLKKRWQLALSK |
| L10 | KKRWQLALSKGYHFY |
| L11 | LALSKGVHFVCNLLL |
| L12 | GVHFVCNLLLLFVTV |
| L13 | CNLLLLFVTVYSHLL |
| L14 | LFVTVYSHLLLVAAG |
| L15 | VSHLLLVAAGLEAPF |
| L16 | LVAAGLEAPFLYLYA |
| L17 | LEAPFLYLYALVYFL |
| L18 | LVLYALVYFLQSINF |
| L19 | LVYFLQSINFVRIIM |
| L20 | QSINFVRIIMRLWLC |
| L21 | VRIIMRLWLCWKCRS |
| L22 | RLWLCWKCRSKNPLL |
| L23 | WKCRSKNPLLYDANY |
| L24 | KNPLLYDANYFLCWH |
| M1 | YDANYFLCWHTNCYD |
| M2 | FLQWHTNCYDYCIPY |
| M3 | TNCYDYDIPYNSVTS |
| M4 | YCIPYNSVTSSIVTT |
| M5 | NSVTSSIVTTSGDGT |
| M6 | SIVITSGDGTTSPIS |
| M7 | SGDGTTSPISEHDYQ |
| M8 | TSPISEHDYQIGGYT |
| M9 | [symbol] |
| M10 | IGGYTEKWESGVKDC |
| M11 | EKWESGVKDCVVLHS |
| M12 | GVKDCVVLHSYFTSD |
| M13 | VVLHSYFTSDYYQLY |
| M14 | VFTSDYYQLYSTQLS |
| M15 | YYQLYSTQLSTDTGV |
| M16 | STQLSTDTGVEHVTF |
| M17 | TDTGVEHVTFFIYNK |
| M18 | EHVTFFIYNKTVDEP |
| M19 | FIYNKIVDEPEEHVQ |
| M20 | IVDEPEEHVQIHTID |
| M21 | EEHVQIHTIDGSSGV |
| M22 | IHTIDGSSGVVNPVM |
| M23 | GSSGVVNPVMEPIVD |
| M24 | NVPVMEPIVDEPTTT |
| N1 | EPIYDEPTTTTSVPL |
| N2 | |
| N3 | |
| N4 | NADSNGTITVEELKK |
| N5 | GTITVEELKKLLEQW |
| N6 | EELKRLLEQWNLVIG |
| N7 | LLEQWNLVIGFLFLT |
| N8 | [symbol] |
| N9 | FLFLTWICLLQFAYA |
| N10 | WICLLQFAYANRNRF |
| N11 | QFAYANRNRFLYIIK |
| N12 | NRNRFLYIIKLIFLW |
| N13 | LYIIKLIFLWLLWPV |
| N14 | LIFLWLLWPVTLACF |
| N15 | LLWPVTLACFVLAAV |
| N16 | TLACFVLAAVYTINW |
| N17 | VLAAVYRINWTTGGT |
| N18 | YRINWTTGGTATANA |
| N19 | ITGGIAIANACLVGL |
| N20 | AIAMACLVGLMWLSY |
| N21 | CLVGLMWLSYFIASF |
| N22 | MWLSYFIASFHLFAR |
| N23 | FIASFRLFARTRSMW |
| N24 | RLFARTRSMWSFNPE |
| O1 | TRSMWSFNPETNILL |
| O2 | SFNPETNILLNVPLH |
| O3 | TNILLNVPLHGTILT |
| O4 | NVPLHGTILTRPLLE |
| O5 | GTILTRPLLESELVI |
| O6 | RPLLESELVIGAVIL |
| O7 | SELVIGAVILRGHLR |
| O8 | GAVILRGHLRIAGHM |
| O9 | RGHLRIAGHHLGRCD |
| O10 | IAGHHLGRCDIKDLP |
| O11 | LGRCDIKDLPKEITV |
| O12 | IKDLPKEITVATSRT |
| O13 | KETTVATSRTLSYYK |
| O14 | ATSRTLSYYKLGASQ |
| O15 | LSYYKLGASQRVAGD |
| O16 | LGASQHVAGDSGFAA |
| O17 | RVAGDSGFAAVSNVR |
| O18 | SGFAAYSHYRIGNYK |
| O19 | YSRYRIGNYKLNTDH |
| O20 | IGNVKLNTDHSSSSD |
| O21 | LNTDHSSSSDNIALL |
| O22 | TDHSSSSDNIALLVQ |
| O23 | |
| O24 | |
| P1 | NFHLVDFQVTIAEIL |
| P2 | DFQVTIAEILLIIMR |
| P3 | IAEILLIIMRTFKVS |
| P4 | LIIMRTFKVSIWNLD |
| P5 | TFKVSIWDLDYIINL |
| P6 | IWNLDYIINLIIKNL |
| P7 | VIINLIIKNLSKSLT |
| P8 | IIKNLSKSLTENKYS |
| P9 | SKSLTENKYSQLDEE |
| P10 | ENKYSQLDEEQPMIE |
| P11 | NKVSQLDEEQPMEID |
| P12 | |
| P13 | |
| P14 | MKIILFLALITLATC |
| P15 | FLALITLATCELYHY |
| P16 | TLATCELYHVQECVR |
| P17 | ELYHYQECVRGTTVL |
| P18 | QECVRGTTVLLKEPC |
| P19 | GTTVLLKEPCSSGTY |
| P20 | LKEPCSSGTYEGNSP |
| P21 | SSGTYEGNSPFHPLA |
| P22 | EGNSPFHPLADNKFA |
| P23 | FHPLADNKFALTCFS |
| P24 | DNKFALTCFSTQFAF |
| Q 1 | LTCFSTQFAFACPDG |
| Q 2 | TQFAFACPDGVKHVY |
| Q 3 | ACPDGVKHVYQLRAR |
| Q 4 | VKHVYQLRARSVSPK |
| Q 5 | QLRARSVSPKLFIRQ |
| Q 6 | SVSPKLFIRQEEVQE |
| Q 7 | LFIRQEEVQELYSPI |
| Q 8 | EEVQELYSPIFLIVA |
| Q 9 | LYSPIFLIVAAIYFI |
| Q10 | FLIVAAIVFITLCFT |
| Q11 | AIVFTTLCFTLKRKT |
| Q12 | IVFITLCFTLKRKTE |
| Q13 | |
| Q14 | |
| Q15 | MKFLVFLGIITTVAA |
| Q16 | FLGIITTVAAFHQEC |
| Q17 | TTVAAFHQECSLQSC |
| Q18 | FHQECSLQSCTQHQP |
| Q19 | SLQSCTQHQPYVVDD |
| Q20 | TQHQPYVVDDPCPIH |
| Q21 | YVVDDPCPIHFYSKW |
| Q22 | PCPIHFVSKWYIRVG |
| Q23 | FYSKWYIRVGARKSA |
| Q24 | YIRVGARKSAPLIEL |
| R 1 | ARKSAPLIELCFDEA |
| R 2 | PLIELCVDEAGSKSP |
| R 3 | CVDEAGSKSPIQYID |
| R 4 | GSKSPIQYIDIGNYT |
| R 5 | IQYIDIGNYTVSCLP |
| R 6 | IGNVTVSCLPFTINC |
| R 7 | VSCLPFTINCQEPKL |
| R 8 | FTINCQEPKLGSLVV |
| R 9 | QEPKLGSLVVRCSFY |
| R10 | GSLVVRCSFYEDFLE |
| R11 | RCSFYEDFLEYHDVR |
| R12 | EDFLEYHDVRVVLDF |
| R13 | DFLEYHDVRVVLDFI |
| R14 | |
| R15 | |
| R16 | MSDNGPQNQRNAPRI |
| R17 | PQNQRNAPRITFGGP |
| R18 | NAPRITFGGPSDSTG |
| R19 | TFGGPSDSTGSNQNG |
| R20 | SDSTGSNQNGERSGA |
| R21 | SNQNGERSGARSKQR |
| R22 | ERSGARSKQRRPQGL |
| R23 | RSKQRRPQGLPNNTA |
| R24 | RPQGLPNNTASWFTA |
| S 1 | PNNTASWFTALTQHG |
| S 2 | SWFTALTQHGKEDLK |
| S 3 | LTQHGKEDLKFPRGQ |
| S 4 | KEDLKFPRGQGVPIN |
| S 5 | FPRGQGVPINTNSSP |
| S 6 | GVPINTNSSPDDQIG |
| S 7 | TNSSPDDQIGYYRRA |
| S 8 | DDQIGYYRRATRRIR |
| S 9 | YYRRATRRIRGGDGK |
| S10 | [symbol] |
| S11 | GGDGKMKDLSPRWYF |
| S12 | MKDLSPRWYFYYLGT |
| S13 | PRWYFYYLGTGPEAG |
| S14 | YYLGTGPEAGLPYGA |
| S15 | GPEAGLPYGANKDGI |
| S16 | LPYGANKDGIIWVAT |
| S17 | NKDGIIWVATEGALN |
| S18 | IWVATEGALNTPKDH |
| S19 | EGALNTPKDHIGTRN |
| S20 | TPKDHIGTRNPANNA |
| S21 | IGTRNPANNAAIVLQ |
| S22 | PANNAAIVLQLPQGT |
| S23 | AIVLQLPQGTTLPKG |
| S24 | LPQGTTLPKGFYAEG |
| T 1 | TLPKGFYAEGSRGGS |
| T 2 | FYAEGSRGGSQASSR |
| T 3 | SRGGSQASSRSSSRS |
| T 4 | QASSRSSSRSRNSSR |
| T 5 | SSSRSRNSSRNSTPG |
| T 6 | RNSSRNSTPGSSRGT |
| T 7 | NSTPGSSRGTSPARM |
| T 8 | SSRGTSPARMAGNGG |
| T 9 | SPARMAGNGGDAALA |
| T10 | AGNGGDAALALLLLD |
| T11 | DAALALLLLDRLNQL |
| T12 | LLLLDRLNQLESKMS |
| T13 | RLNQLESKMSGKGQQ |
| T14 | ESKMSGKGQQQQGQT |
| T15 | GKGQQQQGQTVTKKS |
| T16 | QQGQTVTKKSAAEAS |
| T17 | VTKKSAAEASKKPRQ |
| T18 | AAEASKKPRQKRTAT |
| T19 | KKPRQKRTATKAYNV |
| T20 | KRTATKAYVNPQAFG |
| T21 | KAYNVTQAFGRRGPE |
| T22 | TQAFGRRGPEQTQGN |
| T23 | RRGPEQTQGNFGDQE |
| T24 | QTQGNFGDQELIRQG |
| U 1 | FGDQELIRQGTDYKH |
| U 2 | LIRQGTDYKHWPQIA |
| U 3 | TDYKHWPQIAQFAPS |
| U 4 | WPQAIQFAPSASAFF |
| U 5 | QEAPSASAFFGMSRI |
| U 6 | ASAFFGMSRIGMEVT |
| U 7 | GMSRIGMEVTPSGTW |
| U 8 | GMEVTPSGTWLTYTG |
| U 9 | OSGTWLTYTGAIKLD |
| U10 | LTYTGAIKLDDKDPN |
| U11 | AIKLDDKDPNFKDQV |
| U12 | DKDPNFKDQVILLNK |
| U13 | FKDQVILLNKHIDAY |
| U14 | ILLNKHIDAYKTFPP |
| U15 | HIDAYKTFPPTEPKK |
| U16 | KTFPPTEPKKDKKKK |
| U17 | TEPKKDKKKKADETQ |
| U18 | DKKKKADETQALPQR |
| U19 | ADETQAPGQRQKKQQ |
| U20 | ALPQRQKKQQTVTLL |
| U21 | QKKQQTVTLLPAADL |
| U22 | TVTLLPAADLDDFSK |
| U23 | PAADLDDFSKQLQQS |
| U24 | DDFSKQLQQSMSSAD |
| V 1 | KQLQQSMSSADSTQA |
| V 2 | |
| V 3 | |
| V 4 | MYSFVSEETGTLIVN |
| V 5 | SEETGTLIVNSVLLF |
| V 6 | TLIVNSVLLFLAFVV |
| V 7 | SVLLFLAFVVFLLVT |
| V 8 | LAFVVFLLVTLAILT |
| V 9 | FLLVTLAILTALRLC |
| V 10 | LAILTALRLCAYCCN |
| V 11 | ALRLCAYCCNIVNVS |
| V 12 | AYCCNIVNVSLVKPS |
| V 13 | IVNVSLVKPSFYVYS |
| V 14 | LVKPSFYVYSRVKNL |
| V 15 | FYVYSRVKNLNSSRV |
| V 16 | RVKNLNSSRVPDLLV |
| V 17 | |
| V 18 | |
| V 19 | MGYINVFAFPFTIYS |
| V 20 | VFAFPFIIYSLLLCR |
| V 21 | FTIYSLLLCRMNSRN |
| V 22 | LLLSRMNSRNYTAQV |
| V 23 | MNSRNYIAQVDVVNF |
| V 24 | RNVIAQVDVVNFNLT |

[symbol]

indicates data missing or illegible when filed

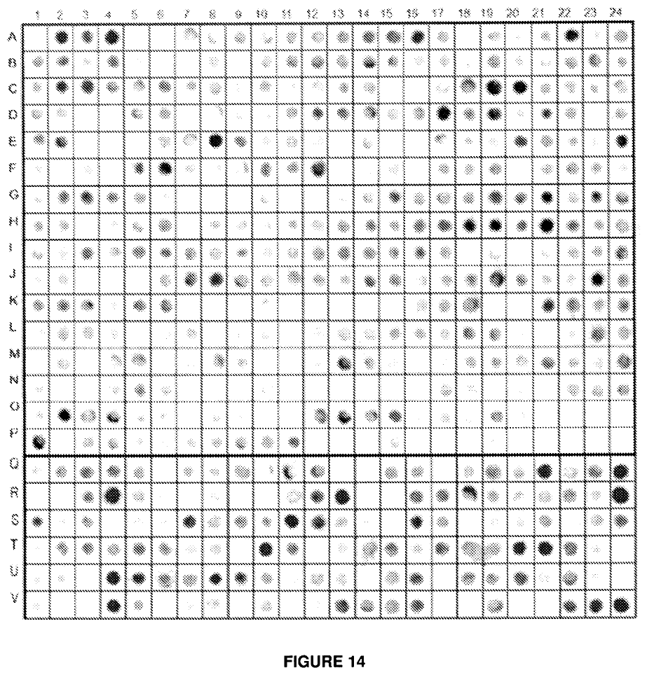

TABLE 20
List of SARS-COV-2 polyamino acids
synthesized for mapping IgA-reactive
epitopes from patient sera in FIG. 14.

| Spot | Polypeptide |
|---|---|
| A1 | IHLVNNESSEVIVHK |
| A2 | GYPKDGNAFNNLDRI |
| A3 | KEVPALTAVETGATN |
| A4 | YPYDVPDYAGYPYDV |
| A5 | |
| A6 | MFVFLVLLPLVSSQC |
| A7 | VLLPLVSSQCVNLTT |
| A8 | VSSQCVNLTTRTQLP |
| A9 | VNLTTRTQLPPAYTN |
| A10 | RTQLPPAYTNSFTRG |
| A11 | PAYTNSFTRGVVYPD |
| A12 | SFTRGVYYPDKVFRS |
| A13 | VYYPDKVFRSSVLHS |
| A14 | NVFRSSVLHSTQDLF |
| A15 | SVLHSTQDLFLPFFS |
| A16 | [symbol] |
| A17 | LPFFSNVTWFHAIHV |
| A18 | NVTWFHAIHVSGTNG |
| A19 | HAIHVSGTNGTKRFD |
| A20 | SGTNGTKRFDNPVLP |
| A21 | TKRFDNPVLPFNDGV |
| A22 | NPVLPFNDGVYFAST |
| A23 | FNDGVYFASTEKSNI |
| A24 | YFASTEKSNIIRGWI |
| B1 | EKSNIIRGWIFGTTL |
| B2 | IRGWIFGTTLDSMTQ |
| B3 | FGTTLDSNTQSLLIV |
| B4 | DSKTQSLLIVNNATN |
| B5 | SLLIVNNATNVVIKV |
| B6 | NNATNVVIKVCEFQF |
| B7 | VVIKVCEFQFCNDPF |
| B8 | CEFQFCNDPFLGVYY |
| B9 | CNDPFLGVYYHKNNK |
| B10 | LGVYYHKNNKSWMES |
| B11 | HKNNKSWMESEFRVY |
| B12 | SWMESEFRYVSSANN |
| B13 | EFRVYSSANNCTFEY |
| B14 | SSANNCTFEYVSQPF |
| B15 | CTFEYVSQPFLMDLE |
| B16 | YSQPFLMDLEGKQGN |
| B17 | LMDLEGKQGNFKNLR |
| B18 | GKQGNFKNLREFVFK |
| B19 | FKNLREFVFKNIDGY |
| B20 | EFVFKNIDGYFKIYS |
| B21 | NIDGYFKIYSKHTPI |
| B22 | FKIVSKHTPINLVRD |
| B23 | KHTPINLVRDLPQFG |
| B24 | NLVRDLPQGFSALEP |
| C1 | LPQGFSALEPLVDLP |
| C2 | SALEPLVDLPIGINI |
| C3 | LVDLPIGINITRFQT |
| C4 | IGINITRFQTLLALH |
| C5 | YRFQTLLADHRSYLT |
| C6 | LLALHRSYLTPGDSS |
| C7 | RSYLTPGDSSSGWTA |
| C8 | PGDSSSGWTAGAAAY |
| C9 | SGWTAGAAAYYVGYL |
| C10 | GAAAYYVGYLQPRTF |
| C11 | YVGYLQPRTFLLKYN |
| C12 | QPRTFLLKYNENGTI |
| C13 | LLKYNENGTITDAVD |
| C14 | ENGTITDAVDCALDP |
| C15 | TDAVDCALDPLSETK |
| C16 | CALDPLSETKCTLKS |
| C17 | LSETKCTLKSFTVEK |
| C18 | CTLKSFTVEKGIVQT |
| C19 | FTVEKGIYQTSNFRV |
| C20 | GIYQTSNFRVQPTES |
| C21 | SNFRVQPTESIVRFS |
| C22 | QPTESIVRFPNITNL |
| C23 | IVRFPNITNLCPFGE |
| C24 | NITNLCPFGEVFNAT |
| D1 | CPFGEVFNATRFASV |
| D2 | VFNATRFASVYAWNR |
| D3 | RFASVYAWNRKRISN |
| D4 | YAWNRKRISNCVADY |
| D5 | NRISNCVADYSVLVN |
| D6 | CVADYSVLVNSASFS |
| D7 | SVLVNSASFSTFKCY |
| D8 | SASGSTFKCYGVSPT |
| D9 | TFKCYGVSPTKLNDL |
| D10 | GVSPTKLNDLCFTNV |
| D11 | KLNDLCFTNVYADSF |
| D12 | CFTNVYADSFVIRGD |
| D13 | YADSFVIRGDEVRQI |
| D14 | VIRGDEVRQIAPGQT |
| D15 | EVRQIAPGQTGKIAD |
| D16 | APGQTGKIADYNYKL |
| D17 | GKIADYNYKLPDDFT |
| D18 | YNYKLPDDFTGCVIA |
| D19 | PDDFTGCVIAWNSNN |
| D20 | GCVNAWNSNNLDSKV |
| D21 | WNSNNLDSKVGGNVN |
| D22 | LDSKVGGNVNYLVRL |
| D23 | GGNYNYLVRLFRKSN |
| D24 | YLYRLFRKSNLKPFE |
| E1 | FRKSNLKPFERDIST |
| E2 | LKPFERDISTEIYQA |
| E3 | RDISTEIYQAGSTPC |
| E4 | EIYQAGSTPCNGVEG |
| E5 | GSTPCNGVEGFNCYF |
| E6 | NGVEGFNCYFPLQSY |
| E7 | FNCYFPLQSYGFQPT |
| E8 | PLQSYGFQPTNGVGY |
| E9 | GFQPTNGVGYQFYRV |
| E10 | NGVGYQFYRVVVLSF |
| E11 | QFYRVVVLSFELLHA |
| E12 | VVLSFELLHAPATVC |
| E13 | ELLHAPATVCGPKKS |
| E14 | PATMCGPKKSTNLVK |
| E15 | GPKKSTNLVKNKCVN |
| E16 | TNLVKNKCVNFNFNG |
| E17 | NKCVNFNFNGLTGTG |
| E18 | FNFNGLTGTGVLTES |
| E19 | LTGTGVLTESNKKFL |
| E20 | VLTESNKKFLPFQQF |
| E21 | NKKFLPFQQFGRDIA |
| E22 | PFQQFGRDIADTTDA |
| E23 | GRDIAGTTDAYRDPQ |
| E24 | DTTDAVRDPQTLEIL |
| F1 | VRDPQTLEILDITPC |
| F2 | TLEILDITPCSFGGV |
| F3 | DITPCSFGGVSVIIP |
| F4 | SFGGVSVITPGTNTS |
| F5 | SVITPGTNTSNQVAV |
| F6 | GTNTSNQVAVLYQDV |
| F7 | NQVAVLYQDVNCTEV |
| F8 | LYQDVNCTEVPVAIH |
| F9 | NCTEVPVAIHADQLT |
| F10 | PVADHADQLTPTWRY |
| F11 | ADQLTPTWRVYSTGS |
| F12 | PTWRVVSTGSNCFQT |
| F13 | VSTGSNVFQTRAGCL |
| F14 | NVFQTRAGCLIGAEH |
| F15 | RAGCLIGAEHVNNSY |
| F16 | IGAEHVNNSYECDIP |
| F17 | YNNSYECDIPIGAGI |
| F18 | ECDIPIGAGICASYQ |
| F19 | IGAGICASYQTGTNS |
| F20 | CASYQTQTNSPRRAR |
| F21 | TQTNSPRRARSVASQ |
| F22 | PRRARSVASQSIIAY |
| F23 | SVASQSIIAYTMSLG |
| F24 | SIIAYTMSLGAENSV |
| G1 | TMSLGAENSVAYSNN |
| G2 | AENSVAYSNNSIAIP |
| G3 | AYSNNSIAIPTNFTI |
| G4 | STAIPTNFTISVTTE |
| G5 | TNFTISVTTEILPVS |
| G6 | SVTTEILPVSMTKTS |
| G7 | ILPVSMTKTSVDCTM |
| G8 | MTKTSVDCTMYICGD |
| G9 | VDCTMYICGDSTECS |
| G10 | YICGDSTECSNLLLQ |
| G11 | STECSNLLLQYGSFC |
| G12 | NLLLQYGSFCTQLNR |
| G13 | YGSFCTQLNRALTGI |
| G14 | TQLRNALTGIAVEQD |
| G15 | ALTGIAVEQDKNTQI |
| G16 | AVEQDKNTQEVFAQV |
| G17 | KNTQEVFAQVKQIYK |
| G18 | VFAQVKQIYKTPPIK |
| G19 | KQIYKTPPIKDFGGF |
| G20 | TPPIKDFGGFNFSQI |
| G21 | DFGGFNFSQILPDPS |
| G22 | NFSQILPDPSKPSKR |
| G23 | LPDPSKPSKRSFIED |
| G24 | KPSKRSFIEDLLFNK |
| H1 | SFIEDLLFNKVTLAD |
| H2 | LLFNKVTLADAGFIK |
| H3 | VTLADAGFIKQVGDC |
| H4 | AGFIKQVGDCLGDIA |
| H5 | QYGDCLGDIAARDLI |
| H6 | LGDIAARDLTCAQKF |
| H7 | ARDLICAQKFNGLTV |
| H8 | CAQKFNGLTVLPPLL |
| H9 | NGLTVLPPLLTDEMI |
| H10 | LPPLLTDEMIAQYTS |
| H11 | TDEMIAQYTSALLAG |
| H12 | AQYTSALLAGTTTSG |
| H13 | ALLAGTITSGWTFGA |
| H14 | TITSGWTFGAGAALQ |
| H15 | WTFGAGAALQIPFAM |
| H16 | GAALQPIFAMQMAYR |
| H17 | IPFAMQMAYRFNGIG |
| H18 | QMAYRFNGIGVTQNV |
| H19 | FNGIGVTQNVLYENQ |
| H20 | VTQNVLYENQKLIAN |
| H21 | LYENQKLIANQFNSA |
| H22 | KLIANQFNSAIGKIQ |
| H23 | QFNSAIGKIQDSLSS |
| H24 | IGKIQDSLSSTASAL |
| I1 | DSLSSTASALGKLQD |
| I2 | TASALGKLQDVVNQN |
| I3 | GKLQDVVNQNAQALN |
| I4 | VVNQNAQALNTLVKQ |
| I5 | AQALNTLVKQLSSNF |
| I6 | TLVKQLSSNFGAISS |
| I7 | LSSNFGAISSVLNDI |
| I8 | GAISSVLNDILSRLD |
| I9 | VLNDILSRLDKVEAE |
| I10 | LSRLDKVEAEVQIDR |
| I11 | KVEAEVQIDRLITGR |
| I12 | VQIDALITGRLQSLQ |
| I13 | LITGRLQSLQTYVTQ |
| I14 | LQSLQTYVTQQLIRA |
| I15 | TYVTQQLTRAAEIRA |
| I16 | QLIRAAEIRASANLA |
| I17 | AEIRASANLAATKMS |
| I18 | SANLAATKMSECVLG |
| I19 | ATKMSECVLGQSKRV |
| I20 | ECVLGQSKRVDFCGK |
| I21 | QSKRVDFCGKGYHLM |
| I22 | DFCGKGYHLMSFPQS |
| I23 | GYHLMSFPQSAPHGV |
| I24 | SFPQSAPHGVVFLHV |
| J1 | APHGVVFLHVTVVPA |
| J2 | VFLHVTYVPAQEKNF |
| J3 | TYVPAQEKNFTTAPA |
| J4 | QEKNFTTAPAICHDG |
| J5 | TTAPAICHDGKAHFP |
| J6 | ICHDGKAHFPREGVF |
| J7 | KAHFPREGVFVSNGT |
| J8 | REGVFVSNGTHWFVT |
| J9 | VSNGTHWFVTQRNFY |
| J10 | HWFVTQRNFYEPQII |
| J11 | QRNFYEPQIITTDNT |
| J12 | EPQIITTDNTFVSGN |
| J13 | TTDNTFVSGNCDVVI |
| J14 | FVSGNCDVVIGIVNN |
| J15 | CDVVIGIVNNTVYDP |
| J16 | GIVNNTVYDPLQPEL |
| J17 | TVYDPLQPELDSFKE |
| J18 | LQPEDLSFKEELDKY |
| J19 | DSFKEELDKYFKNHT |
| J20 | ELDKYFKNHTSPDVD |
| J21 | FKNHTSPDVDLGDIS |
| J22 | SPDVDLGDISGINAS |
| J23 | LGDISGINASVVNIQ |
| J24 | GINASVVNIQKEIDR |
| K1 | VVNIQKEIDRLNEVA |
| K2 | KEIDRLNEVAKNLNE |
| K3 | LNEVAKNLNESLTDL |
| K4 | KNLNESLIDLQELGK |
| K5 | SLIDLQELGKYEQYI |
| K6 | QELGKYEQYIKWPWY |
| K7 | YEQYIKWPWYIWLGF |
| K8 | KWPWYIWLGFIAGLI |
| K9 | IWLGFIAGLIAIVMV |
| K10 | IAGLIAIVMVTIMLC |
| K11 | AIVMVTIMLCCMTSC |
| K12 | TIMLCCMTSCCSCLK |
| K13 | CMTSCCSCLKGCCSC |
| K14 | CSCLKGCCSCGSCCK |
| K15 | GCCSCGSCCKFDEDG |
| K16 | GSCCMFDEDDSEPVL |
| K17 | FDEDDSEPVLKGVKL |
| K18 | DDSEPVLKGVKLHVT |
| K19 | |
| K20 | |
| K21 | MDLFMRIFTIGTVTL |
| K22 | RIFTIGTVTLKQGEI |
| K23 | GTVTLKQGEIKDATP |
| K24 | KQGEIKDATPSDFVR |
| L1 | KDATPSDFVRATATI |
| L2 | SDFVRATATIPIQAS |
| L3 | ATATIPIQASLPFGW |
| L4 | PIQASLPFGWLIVGV |
| L5 | LPFGWLIVGVALLAV |
| L6 | LIVGVALLAVFQSAS |
| L7 | ALLAVFQSASKIITL |
| L8 | FQSASKIITLKKRWQ |
| L9 | KIITLKKRWQLALSK |
| L10 | KKRWQLALSKGVHFV |
| L11 | LALSKGVHFVCNLLL |
| L12 | GVHFVCNLLLLFVTV |
| L13 | CNLLLLFVTVYSHLL |
| L14 | LFVTVYSHLLLVAAG |
| L15 | YSHLLLVAAGLEAPF |
| L16 | LVAAGLEAPFLYLYA |
| L17 | LEAPFLYLYALVYFL |
| L18 | LYLYALVYFLQSINF |
| L19 | LVYFLQSINFVRIIM |
| L20 | QSINFVRIIMRLWLC |
| L21 | VRIIMRLWLCWKCRS |
| L22 | RLWLCWKCRSKNPLL |
| L23 | WKCRSKNPLLYDANY |
| L24 | KNPLLYDANYFLCWH |
| M1 | YDANYFLCWHTNCYD |
| M2 | FLCWHTNCYDYCIPY |
| M3 | TNCYDYCIPYNSVTS |
| M4 | YCIPYNSVTSSIVIT |
| M5 | NSVTSSIVITSGDGT |
| M6 | SIVTTSGDGTTSPIS |
| M7 | SGDGTTSPISEHDYQ |
| M8 | TSPISEHGYQIGGYT |
| M9 | EHDYQIGGYTEKWES |
| M10 | IGGYTEKWESGVKDC |
| M11 | EKWESGVKDCVVLHS |
| M12 | GVKDCVVLHSYFTSD |
| M13 | VVLHSVFTSDYVQLV |
| M14 | YFTSDYYQLYSTQLS |
| M15 | YYQLYSTQLSTDTGV |
| M16 | STQLSTDTGVEHVTF |
| M17 | TDTGVEHVTFFIYNK |
| M18 | EHVTFFIYNKIVDEP |
| M19 | FIYNKIVDEPEEHVQ |
| M20 | IVDPEEEHVQIHTID |
| M21 | EEHVQIHTIDGSSGV |
| M22 | IHTIDGSSGVVNPVM |
| M23 | GSSGVVNPVMEPIYD |
| M24 | VNPVMEPIYDEPTTT |
| N1 | EPIYDEPTTTTSVPL |
| N2 | |
| N3 | |
| N4 | MADSNGTITVEELKK |
| N5 | GTTTVEELKKLLEQW |
| N6 | EELKKLLEQWNLVIG |
| N7 | LLEQWNLVIGFLFLT |
| N8 | NLVIGFLFLTWICLL |
| N9 | FLFLTWICLLQFAYA |
| N10 | WICLLQFAYANRNRF |
| N11 | QFAYANRNRFLYIIK |
| N12 | NRNRFLYIIKLIFLW |
| N13 | LYIIKLIFLWLLWPV |
| N14 | LIFLWLLWPVTLACF |
| N15 | LLWPVTLACFVLAAV |
| N16 | TLACFVLAAVYRINW |
| N17 | VLAAVYRINWITGGI |
| N18 | YRINWITGGIAIAMA |
| N19 | ITGGIAIAMACLVGL |
| N20 | AIAMACLVGLMWLSY |
| N21 | CLVGLMWLSYFIASF |
| N22 | MWLSYFIASFRLFAR |
| N23 | FIASFRLFARTRSMW |
| N24 | RLFARTRSMWSFNPE |
| O1 | TRSMWSFNPETNILL |
| O2 | SFNPETNILLNVPLH |
| O3 | TNILLNVPLHGTILT |
| O4 | NVPLHGTILTRPLLE |
| O5 | GTILTRPLLESELVI |
| O6 | RPLLESELVIGAVIL |
| O7 | SELVIGAVTLRGHLR |
| O8 | GAVILRGHLRTAGHH |
| O9 | RGHLRIAGHHLGRCD |
| O10 | IAGHHLGRCDIKDLP |
| O11 | LGRCDIKDLPKETTV |
| O12 | IKDLPKEITVATSRT |
| O13 | NEITVATSRTLSYYK |
| O14 | ATSRTLSYYKLGASQ |
| O15 | LSYYKLGASQRVAGD |
| O16 | LGASQRVAGDSGFAA |
| O17 | RVAGDSGFAAYSRYR |
| O18 | SGFAAYSRYRIGNYK |
| O19 | YSRYRIGNYKLNDTH |
| O20 | IGNYKLNTDHSSSSD |
| O21 | LNTDHSSSSDNTALL |
| O22 | TDHSSSSDNIALLVQ |
| O23 | |
| O24 | |
| P1 | MFHLVDFQVTIAEIL |
| P2 | DFQVTIAEILLIIMR |
| P3 | IAEILLIIMRTFKVS |
| P4 | LIIMRTFKVSTWNLD |
| P5 | TFKVSTWNLDYIINL |
| P6 | IWNLDYIINLIIKNL |
| P7 | YIINLIIKNLSKSLT |
| P8 | IIKNLSKSLTENKYS |
| P9 | SKSLTENKYSQLDEE |
| P10 | ENKYSQLDEEQPMEI |
| P11 | NKYSQLDEEQPMEID |
| P12 | |
| P13 | |
| P14 | MKIILFLALITLATC |
| P15 | FLALITLATCELYHY |
| P16 | TLATCELYHYQECVR |
| P17 | ELYHYQECVRGTTVL |
| P18 | QECVRGTTVLLKEPC |
| P19 | GTTVLLKEPCSSGTY |
| P20 | LKEPCSSGTYEGNSP |
| P21 | SSGTYEGNSPFHPLA |
| P22 | EGNSPFHPLADNKFA |
| P23 | FHPLADNKFALTCFS |
| P24 | DNKFALTCFSTQFAF |
| Q 1 | LTCFSTQFAFACPDG |
| Q 2 | TQFAFACPDGVKHVY |
| Q 3 | ACPDGVKHVYQLRAR |
| Q 4 | VKHVYQLRARSVSPK |
| Q 5 | QLRARSVSPKLFIRQ |
| Q 6 | SVSPKLFIRQEEVQE |
| Q 7 | LFIRQEEVQELVSPI |
| Q 8 | EEVQELYSPIFLTVA |
| Q 9 | LVSPIFLIVAAIVFI |
| Q10 | FLIVAAIVFITLCFT |
| Q11 | AIVFITLCFTLKRKT |
| Q12 | IVFITLCFTLKRKTE |
| Q13 | |
| Q14 | |
| Q15 | MKFLVFLGIITTVAA |
| Q16 | FLGIITTVAAFHQEC |
| Q17 | TTVAAFHQECSLQSC |
| Q18 | FHQECSLQSCTQHQP |
| Q19 | SLQSCTQHQPYVVDD |
| Q20 | TQHQPYVVDDPCPIH |
| Q21 | YVVDDPCPIHFYSKW |
| Q22 | PCPIHFYSKWYIRVG |
| Q23 | FYSKWYIRVGARKSA |
| Q24 | YIRVGARKSAPLIEL |
| R 1 | ARKSAPLIELCFDEA |
| R 2 | PLIELCVDEAGSKSP |
| R 3 | CVDEAGSKSPIQVID |
| R 4 | GSKSPIQYIDIGNYT |
| R 5 | IQYIDIGNVTVSCLP |
| R 6 | IGNYTVSCLPFTINC |
| R 7 | VSCLPFTINCQEPKL |
| R 8 | FTINCQEPKLGSLVV |
| R 9 | QEPKLGSLVVRCSFY |
| R10 | GSLVVRCSFYEDFLE |
| R11 | RCSFYEDFLEVHDVR |
| R12 | EDFLEYHDVRVVLDF |
| R13 | DFLEYHDVRVVLDFI |
| R14 | |
| R15 | |
| R16 | MSDNGPQNQRNAPRI |
| R17 | PQNQRNAPRITFGGP |
| R18 | NAPRITFGGPSDSTG |
| R19 | TFGGPSDSTGSNQNG |
| R20 | SDSTGSNQNGERSGA |
| R21 | SNQNGERSGARSKQR |
| R22 | ERSGARSKQRRPQGL |
| R23 | RSKQRRPQGLPNNTA |
| R24 | RPQGLPNNTASWFTA |
| S 1 | PNNTASWFTALTQHG |
| S 2 | SWFTALTQHGKEDLK |
| S 3 | LTQHGKEDLKFPRGQ |
| S 4 | KEDLKFPRGQGVPIN |
| S 5 | FPRGQGVPINTNSSP |
| S 6 | GVPINTNSSPDDQIG |
| S 7 | TNSSPDDQIGYYRRA |
| S 8 | DDQIGYYRRATRRIR |
| S 9 | YYRRATRRIRGGDGK |
| S10 | TRRIRGGDGKMKDLS |
| S11 | GGDGKMKDLSPRWVF |
| S12 | MKDLSPRWYFYYLGT |
| S13 | PRWYFYVLGTGPEAG |
| S14 | YYLGTGPEAGLPYGA |
| S15 | GPEAGLPUGANKDGI |
| S16 | LPYGANKDGIIWVAT |
| S17 | NKDGIIWVATEGALN |
| S18 | IWVATEGALNTPKDH |
| S19 | EGALNTPKDHIGTRN |
| S20 | TPKDHIGTRNPANNA |
| S21 | IGTRNPANNAAIVLG |
| S22 | PANNAAIVLQLPQGT |
| S23 | AIVLQLPQGTTLPKG |
| S24 | LPQGTTLPKGFYAEG |
| T 1 | TLPKGFYAEGSRGGS |
| T 2 | FYAEGSRGGSQASSR |
| T 3 | SRGGSQASSRSSSRS |
| T 4 | QASSRSSSRSRNSSR |
| T 5 | SSSRSRNSSRNSTPG |
| T 6 | RNSSRNSTPGSSRGT |
| T 7 | NSTPGSSRGTSPARM |
| T 8 | SSRGTSPARMAGNGG |
| T 9 | SPARMAGNGGDAALA |
| T10 | AGNGGDAALALLLLD |
| T11 | DAALALLLLDRLNQL |
| T12 | LLLLDRLNQLESKMS |
| T13 | RLNQLESKMSGKGQQ |
| T14 | ESKMSGKGQQQQGQT |
| T15 | GKGQQQQGQTVTKKS |
| T16 | QQGQTVTKKSAAEAS |
| T17 | VTKKSAAEASKKPRQ |
| T18 | AAEASKKPRQKRTAT |
| T19 | KKPRQKRTATKAVNV |
| T20 | KRTATKAYNVTQAFK |
| T21 | KAYNVTQAFGRRGPE |
| T22 | RQAFGRRGPEQTQGN |
| T23 | RRGPEQTQGNFGDQE |
| T24 | QTQGNFGDQELIRQG |
| U 1 | FGDQELIRQGTDYKH |
| U 2 | LIRQGTDYKHWPQIA |
| U 3 | TDYKHWPQAIQFAPS |
| U 4 | WPGIAQFAPSASAFF |
| U 5 | QFAPSASAFFGMSRI |
| U 6 | ASAFFGMSRIGMEVT |
| U 7 | GMSRIGMEVTPSGTW |
| U 8 | GMEVTPSGTWLIYTG |
| U 9 | PSGTWLTYTGAIKLD |
| U10 | LTYTGAIKLDDKDPN |
| U11 | AIKLDDKDPNFKDQV |
| U12 | DKPDNFKDQVILLNK |
| U13 | FKDQVILLNKHIDAY |
| U14 | ILLNKHIDAYKTFPP |
| U15 | HIDAYKTFPPTEPKK |
| U16 | KTFPPTEPKKDKKEK |
| U17 | TEPKKDKKKKADETQ |
| U18 | DKKKKADETQALPQR |
| U19 | ADETQALPQRQKKQQ |
| U20 | ALPQRQKKQQTVTLL |
| U21 | QKKQQTVTLLPAADL |
| U22 | TVTLLPAADLDDFSK |
| U23 | PAADLDDFSKQLQQS |
| U24 | DDFSKQLQQSMSSAD |
| V 1 | KQLQQSMSSADSTQA |
| V 2 | |
| V 3 | |
| V 4 | MYSFVSEETGTLIVN |
| V 5 | SEETGTLIVNSVLLF |
| V 6 | TLIVNSVLLFLAFVV |
| V 7 | SVLLFLAFVVFLLVT |
| V 8 | LAFVVFLLVTLAILT |
| V 9 | FLLVTLAILTALRLC |
| V 10 | LATLTALRLCAYCCN |
| V 11 | ALRLCAYCCNTVNVS |
| V 12 | AYCCNIVNVSLVKPS |
| V 13 | IVNVSLVKPSFYVYS |
| V 14 | LVKPSFYVVSRVKNL |
| V 15 | FYVYSRVKNLNSSRV |
| V 16 | RVKNLNSSRVPDLLV |
| V 17 | |
| V 18 | |
| V 19 | MGYINVFAFPFTIYS |
| V 20 | VFAFPFTIYSLLLCR |
| V 21 | FTIYSLLLCRMNSRN |
| V 22 | LLLCRMNSRNVIAQV |
| V 23 | MNSRNYIAQVDVVNF |
| V 24 | RNYIAQVDVVNFNLT |

[symbol]

indicates data missing or illegible when filed

### Example 20—Overview of Libraries of Polyamino Acid Libraries of SARS-CoV-2 on Cellulose Membranes

The libraries polypeptide libraries, described in Example 19, were prepared on cellulose membranes Amino-PEG500-UC540 cellulose membranes, according to the standard SPOT synthesis technique, using an Auto-Spot Robot ASP-222 Auto-Spot Robot according to the manufacturer's instructions. Polyamino acids with a length of 15 residues and an overlap of 10 adjacent residues have been synthesized covering the entire length of the protein.

After synthesis, the free sites of the membranes were blocked with BSA (bovine albumin albumin prepared in TBS-T buffer (50 mM Tris, NaCl; 136 mM, 2 mM KCl; 0.05%, Tween-20; pH 7.4) for 90 min. Next, the membranes were incubated with patient serum (n=3; 1:100, diluted in TBS-T containing 0.75% BSA) and washed for 4× with TBS-T. Subsequently, the membranes were incubated for 1.5 h with goat anti-IgM (mu, KPL), anti-IgG (H+L chain, Thermo Scientific) or human anti-IgA (a chain specific, Calbiochem) IgG antibodies (1:5000, prepared in TB S-T), and then washed with TB S-T and CBS (sodium citrate buffer containing 50 mM NaCl, pH 7.0). Then CDP-Star® chemiluminescent substrate (0.25 mM) with Nitro-Block-II™ Enhancer (Applied Biosystems, USA) was added to complete the reaction.

The chemiluminescent signals were detected on the Odyssey FC equipment (LI-COR Bioscience) and the intensity of the signals quantified using TotalLab TL100 software (v 2009, Nonlinear Dynamics, USA). The data was analyzed with Microsoft Excel program, and only the spots that had signal intensity (SI) greater than or equal to 30% of the highest value obtained in the set of spots in the respective membranes were included in the characterization of the polyamino acids. As a negative control, the background signal intensity of each membrane was used.

### Example 21—Synthesis of Branched Polyamino Acids from SARS-CoV-2

The multiple branched polyamino acids (SARS-X1-SARS-X8) were synthesized using the F-moc solid-phase polyamino acid synthesis strategy on a Schimadzu synthesizer, model PSS8, according to the manufacturer's instructions. Wang Kcore (two-lysine core, K4) resin (Novabiochem) was used as a solid support for the synthesis of branched polyamino acids. The first amino acid to be coupled was the one located in the C-terminal portion of the polypeptide sequence and the last one located at the N-initial. After completion of all cycles of the synthesis of the branched polyamino acids, they were detached from the solid support by treatment with cleavage cocktail (trifluoracetic acid, triisopropylsilane, and ethandiol) according to standard procedure used in the state of the art for production of synthetic polyamino acids and deprotection of protecting groups of the amino acid side chains (Guy and Fields, Methods Emzymol 289, 67-83, 1997). For quality control of the synthesis, each polyamino acid was analyzed by HPLC and MALDI-TOF.

The synthetic polyamino acids X1, X2 and X5 of the SARS-CoV-2 protein S include SEQ ID NO: 100, 101 and 104, respectively. The synthetic polyamino acids X3 and X6 of the N protein of SARS-CoV-2 include the SEQ ID NO: 102 and 105, respectively. The synthetic polyamino acid X4 of SARS-CoV-2 protein E includes SEQ ID NO: 103. The synthetic polyamino acids X7 and X8 of the SARS-CoV-2 protein, encoded by the open reading window (ORFS) between the S and Se genes, include the SEQ ID NO: 106 and 107 respectively.

The genes encoding the X8 protein are found in small open reading frames (ORFs) between the S and Se genes. The genes encoding the ORF6, X4 and X5 proteins are found in the composition of the ORFs between the M and N genes.

A list of synthetic branched polypeptides is shown in Table 21.

TABLE 21
Synthetic branched peptides of
SARS-COV-2 proteins

| | | | SEQ |
|---|---|---|---|
| Code | Sequence | Protein | ID no: |
| SARS-X1 | YFPLQSYGFQPTNGV | S | 100 |
| SARS-X2 | RSYTPGDSSSSSGWTAG | S | 101 |
| SARS-X3 | GKTFPPTEPKKDKKG | N | 102 |
| SARS-X4 | MYSFVSEETGTLIVN | E | 103 |
| SARS-X5 | PLQSYGFQPTNGVGY | S | 104 |
| SARS-X6 | GGMKDLSPRWYFGGG | N | 105 |
| SARS-X7 | GSKSPIQYIDGGGGG | ORF8 | 106 |
| SARS-X8 | YIRGARKSAPLIELG | ORF8 | 107 |

### Example 22—Human Serum Sample Groups

The 134 human serum samples were divided into five groups. The groups were:
Group 0: ten healthy human serum samples obtained before 2016 from blood donor bank (HEMORIO) (sera #1-#10);
Group 1: twenty-six serum samples from “asymptomatic SARS patients”, identified according to the WHO case definition (#1-#26);
Group 2: twenty-four serum samples from “suspected patients” (#27-#50);
Group 3: thirty-eight serum samples from “patients hospitalized for SARS (severe illness)” identified according to the WHO case definition (#51-#88);
Group 4: thirty-six serum samples from “patients immunoprotected for SARS” (#89-#124).
Sera #1-#26, high body temperature with return to normal temperature, RT-PCR+ for SARS-CoV-2, SD (Standard diagnostic Inc.) rapid test for anti-SARS-CoV-2 antibodies negative.
#27-#50: patients with RT-PCR+ diagnosis for SARS-CoV-2 and negative SD rapid test for anti-SARS-CoV-2 antibodies.
#51-#88: hospitalized individuals with exacerbated signs and symptoms of SARS-CoV-2, rapid RT-PCR test +, SD for anti-SARS-CoV-2 antibody positive.
#89-#124 immunoprotected individuals defined as recovered patient, hospitalized or not, who was or was not diagnosed with SARS-CoV-2 positive, sometimes without diagnosis by RT-PCR+, but showing characteristic symptoms.

### Example 23—Identification of SARS-CoV-2 Related IgM Polyamino Acids

In order to identify potential polyamino acids that would be specifically recognized by anti-SARS-CoV-2 IgM antibodies, serum samples from infected patients were analyzed by the SARS-CoV2 spot polypeptide library synthesis array that encompasses all regions of S, ORF3a, M, ORF6, ORF7, ORF8, N, E, ORF10 and control polyamino acids.

Peptide arrays were used to detect the potential binding activity of human sera from infected individuals to polyamino acids. The peptide arrays covered peptide sequences 15 amino acids in length with 10 amino acids overlapping in adjacent spots. Such linear polyamino acids include the following SARS-CoV-2 proteins:
spike protein (S): aa 1-1273 (spots A7-K19),
ORF3a protein (ORF3): aa 1-275 (spots K22-N2),
membrane glycoprotein (M): aa 1-222 (spots N5-023),
ORF6 protein (ORF6): aa 1-61 (spots P2-P12),
ORF7 protein (ORF7): aa 1-121 (spots P15-Q13),
ORF8 protein (ORF8): aa 1-121 (spots Q16-R17),
nucleocapsid protein (N): aa 1-419 (spots R20-V17),
envelope protein (E): aa 1-75 (spots W1-W13)
ORF10 protein (ORF10): aa 1-38 (spots W15-W20)
positive control polyamino acids: A1 and V5 (Clostridium tetani precursor peptide), A2 and V6 (Clostridium tetani precursor peptide), A3 and V7 (human poliovirus peptide), A4 and V8 (triple epitope of hemagglutinin)
non-reactive spots as negative controls.

The serological immune response by human anti-IgM antibody to various SARS-CoV2 synthetic polyamino acids (S, ORF3a, M, ORF6, ORF7, ORF8, N, E, ORF10) and control polyamino acids (Examples 19 and 20) were analyzed using the polyamino acids covalently synthesized on cellulose membrane (spot) and pool (n=3) serum from patients #55, #60 and #74 (group 3), as can be seen in FIG. 12, supplemented by Table 18.

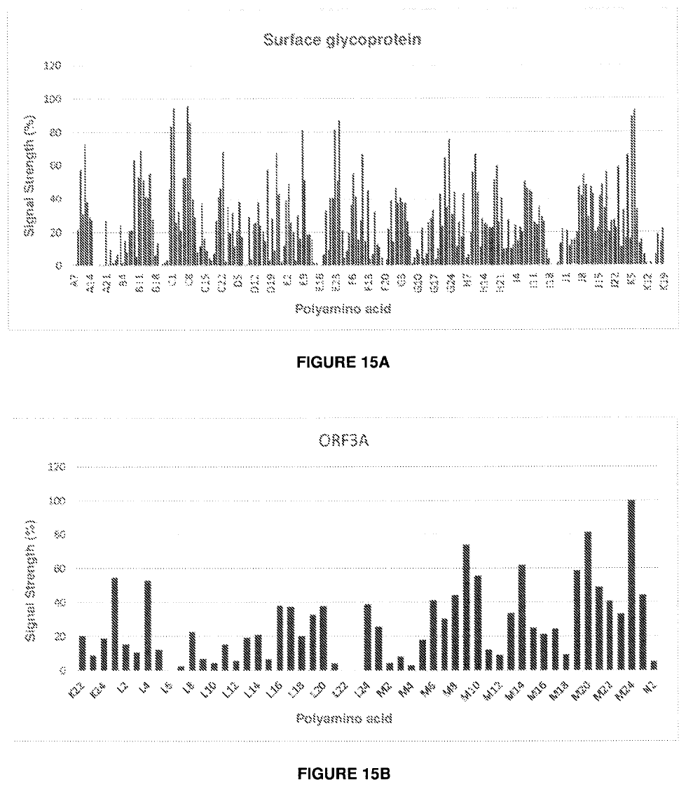

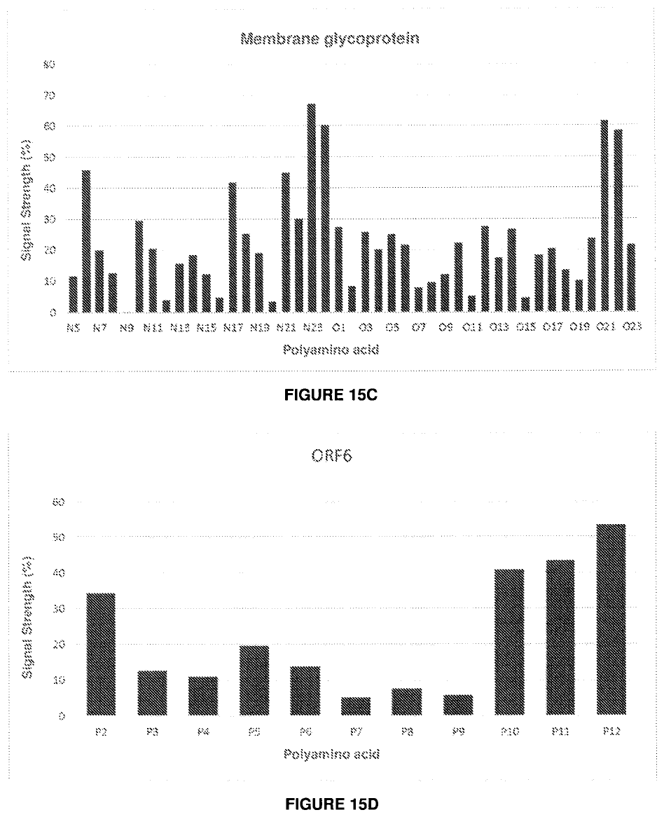

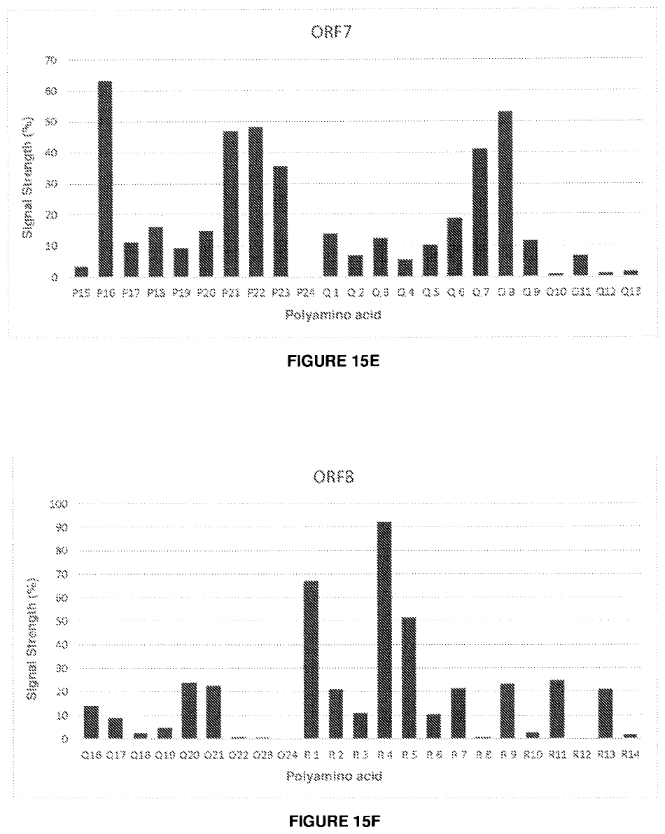

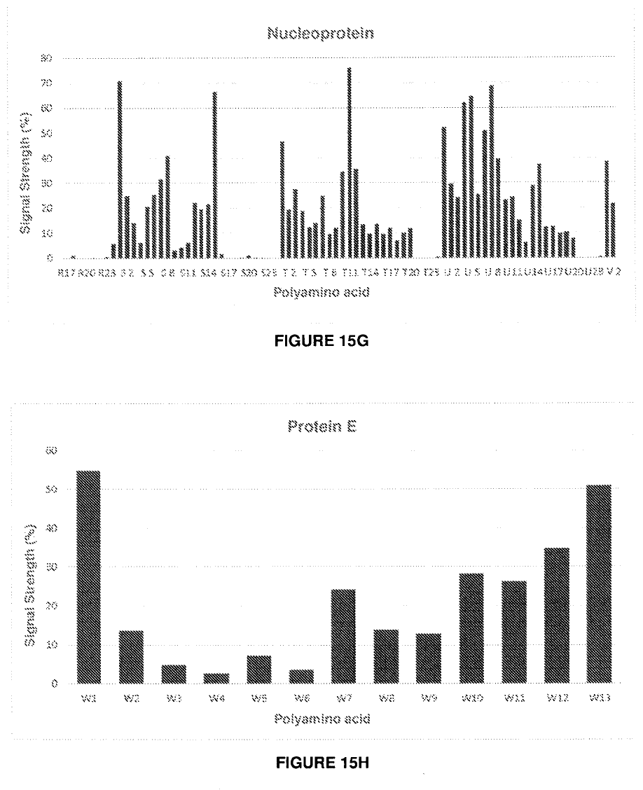

FIGS. 15A to 15I show the signal quantification of the results of the membrane spots from incubation with human sera revealed with goat anti-human IgM secondary antibody.

Human sera have been shown to be significantly reactive to polyamino acids from different viral proteins, as can be seen in FIGS. 15A to 15I, when revealed by human anti-human IgM antibody demonstrating that a large number of different polyamino acids have great potential for diagnosing the disease, even in preliminary stages of infection.

### Example 24—Identification of SARS-Related IgG Epitopes

To identify potential epitopes that would be specifically recognized by anti-SARS-CoV2 IgG antibodies, serum samples from infected patients were analyzed by the SARS-CoV-2 spot polyamino acid library synthesis array that encompasses all regions of S, ORF3a, M, ORF6, ORF7, ORF8, N, E, ORF10 and control polyamino acids.

Peptide arrays were used to detect the potential binding activity of human sera from infected individuals to the polyamino acids. The peptide arrays covered peptide sequences 15 amino acids in length with 10 amino acids overlapping in adjacent Spots. Such linear polyamino acids include the following SARS-CoV-2 proteins:
ORF3a protein (OF3a): aa 1-275 (spots A7-C11),
membrane glycoprotein (M): aa 1-222 (spots C14-E8),
ORF6 protein (OF6): aa 1-61 (spots E11-E21),
ORF7 protein (OF7 (: aa 1-121 (spots E24-F22),
ORF8 protein (OF8): aa 1-121 (spots G1-G23),
spike proteins (S): aa 1-1273 (spots H1-R13),
nucleocapsid protein (N): aa 1-419 (spots R16-V1),
envelope protein (E): aa 1-75 (spots W1-W13),
ORF10 protein (OF10): aa 1-38 (spots W15-W20),
positive control polypeptides: A1 and V4 (Clostridium tetani precursor peptide), A2 and V5 (Clostridium tetani precursor peptide), A3 and V6 (human poliovirus peptide), A4 and V7 (triple epitope of hemagglutinin)
non-reacting spots as negative controls

The serological immune response by IgG antibodies to various SARS-CoV-2 synthetic polyamino acids (ORF3a, M, ORF6, ORF7, ORF8, S, N, E, ORF10) and control polyamino acids were analyzed using the peptides covalently synthesized in the cellulose membrane (spot) and pool (n=3) serum from patients #55, #60 and #74 (group 3), as can be seen in FIG. 13, supplemented by Table 19.

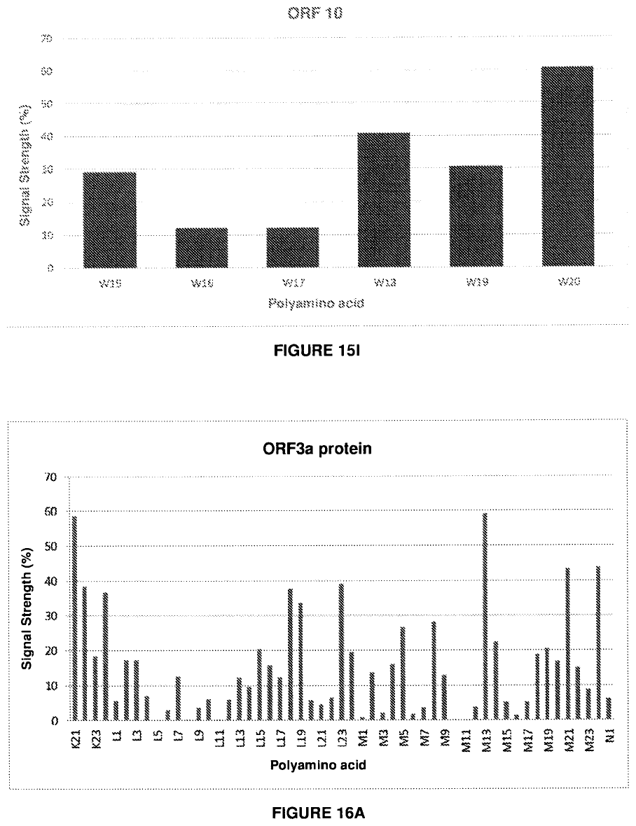

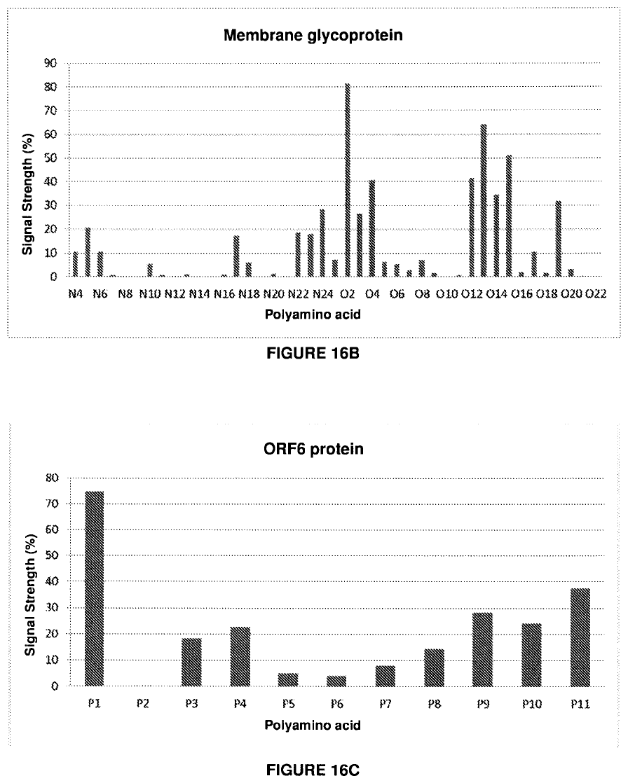

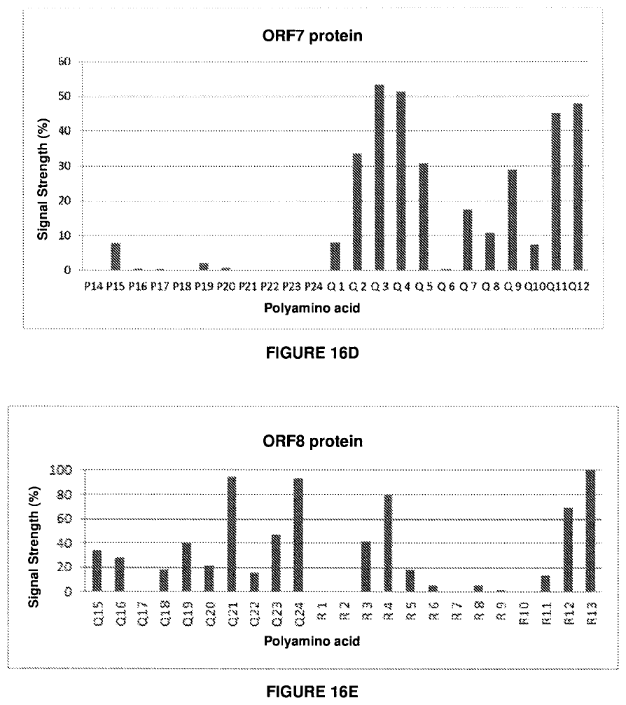

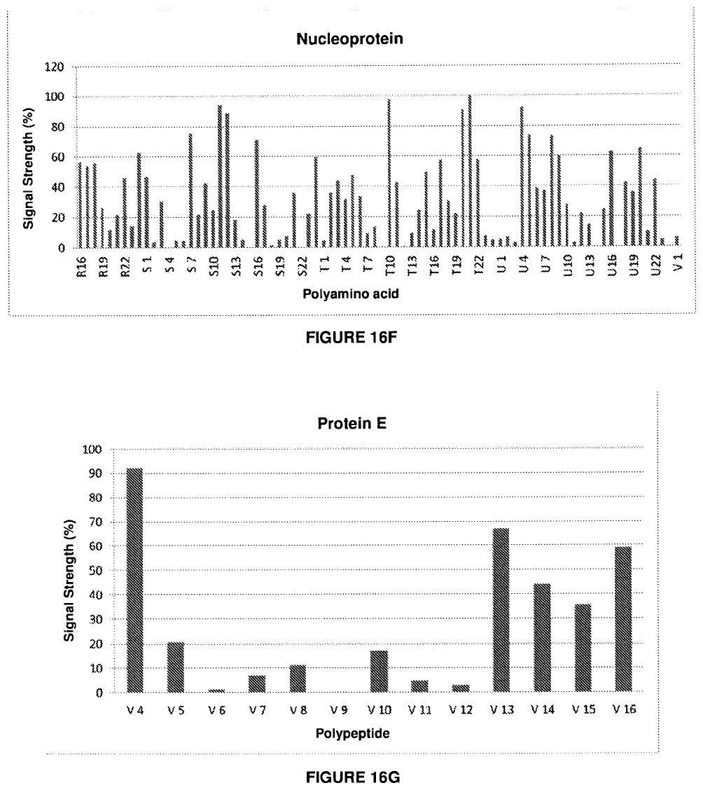

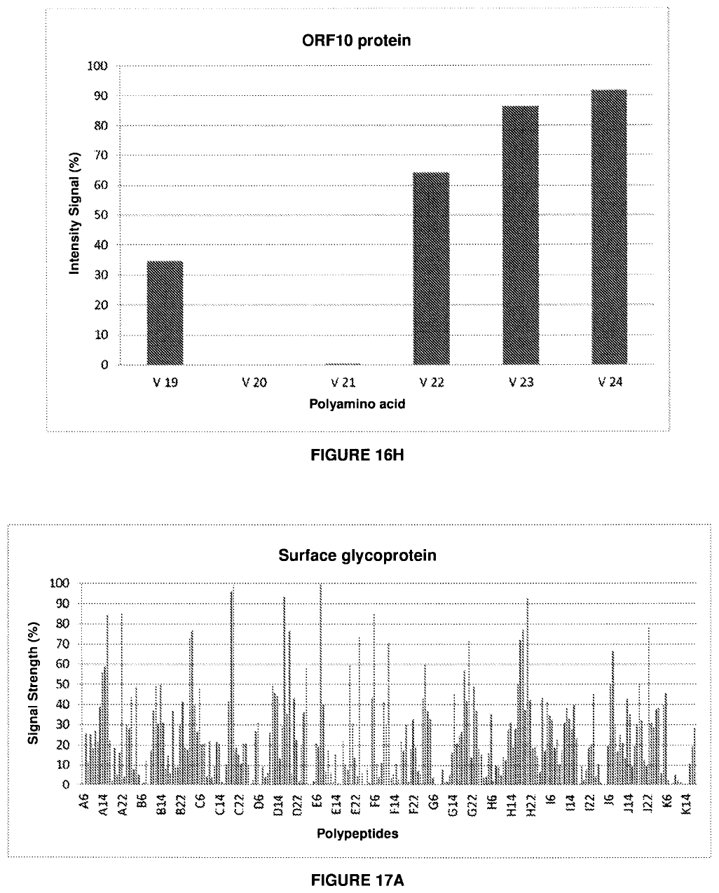

FIGS. 16A to 16H show the signal quantification of the results of the membrane spots from incubation with human sera revealed with goat anti-human IgG secondary antibody.

Human sera have been shown to be significantly reactive to polyamino acids of different viral proteins, as can be seen in FIGS. 16A to 16H, when revealed by human anti-human IgG antibody demonstrating that a large number of different polyamino acids have great potential for diagnosing the disease, even in stages after the acute phase of infection.

### Example 25—Identification of Polyamino Acids IgA Related to SARS

To identify potential polyamino acids that would be specifically recognized by anti-SARS-CoV-2 IgA antibodies, serum samples from infected patients were analyzed by the SARS-CoV-2 spot polyamino acid library synthesis array that encompasses all regions of S, ORF3a, M, ORF6, ORF7, ORF8, N, E, ORF10 and control polyamino acids.

Peptide arrays were used to detect the potential binding activity of human sera from infected individuals to polyamino acids. The peptide arrays covered peptide sequences 15 amino acids in length with 10 amino acids overlapping in adjacent spots. Such linear polyamino acids include the following SARS-CoV-2 proteins:
spike protein (S): aa 1-1273 (spots A6-K18),
ORF3a protein (ORF3): aa 1-275 (spots K21-N1),
membrane glycoprotein (M): aa 1-222 (N4-O22),
ORF6 protein (ORF6): aa 1-61 (P2-P12),
ORF7 protein (ORF7): aa 1-121 (P15-Q13),
ORF8 protein (ORF8): aa 1-121 (Q16-R17),
nucleocapsid protein (N): aa 1-419 (spots R20-V17),
Envelope protein (E): aa 1-75 (spots W1-W-13),
ORF10 protein (ORF10): aa 1-38 (spots W15-W20),
positive control polypeptides: A1 and V4 (Clostridium tetani precursor peptide), A2 and V5 (Clostridium tetani precursor peptide), A3 and V6 (human poliovirus peptide), A4 and V7 (triple epitope of hemagglutinin),
Non-reactor spots as negative controls.

The B cell immune response by IgA antibodies to various synthetic polyamino acids of SARS-CoV-2 (ORF3a, M, ORF6, ORF7, ORF8, S, N, E, ORF10) and control polyamino acids were analyzed using the covalently synthesized peptides in the cellulose membrane (spot) and pool (n=3) serum from patients #55, #60 and #74 (group 3), as can be seen in FIG. 14, supplemented by Table 20.

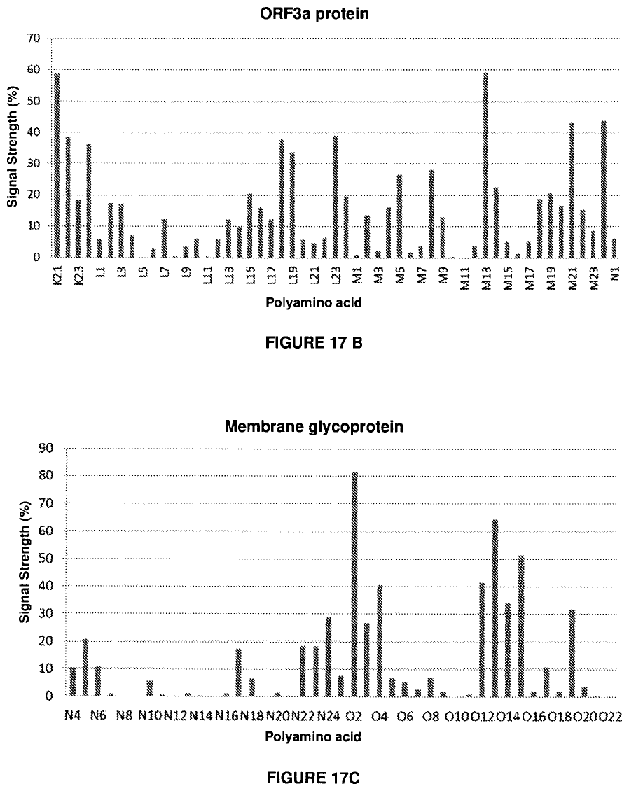

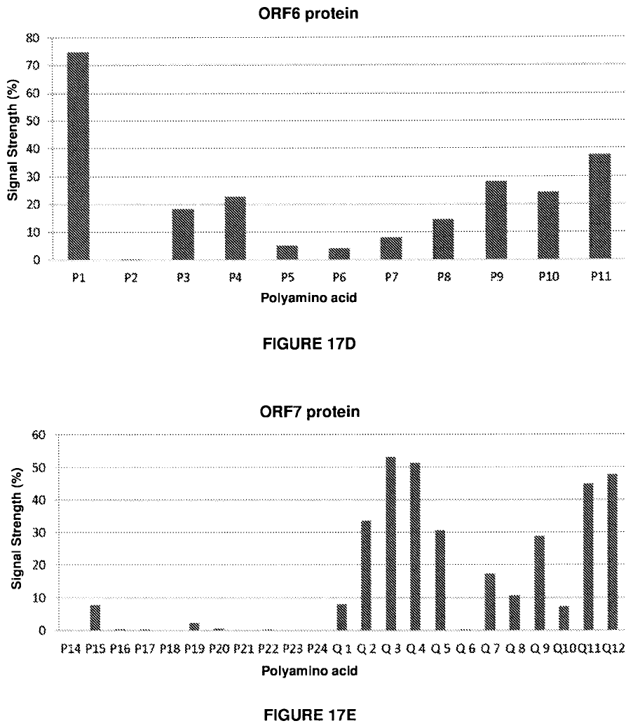

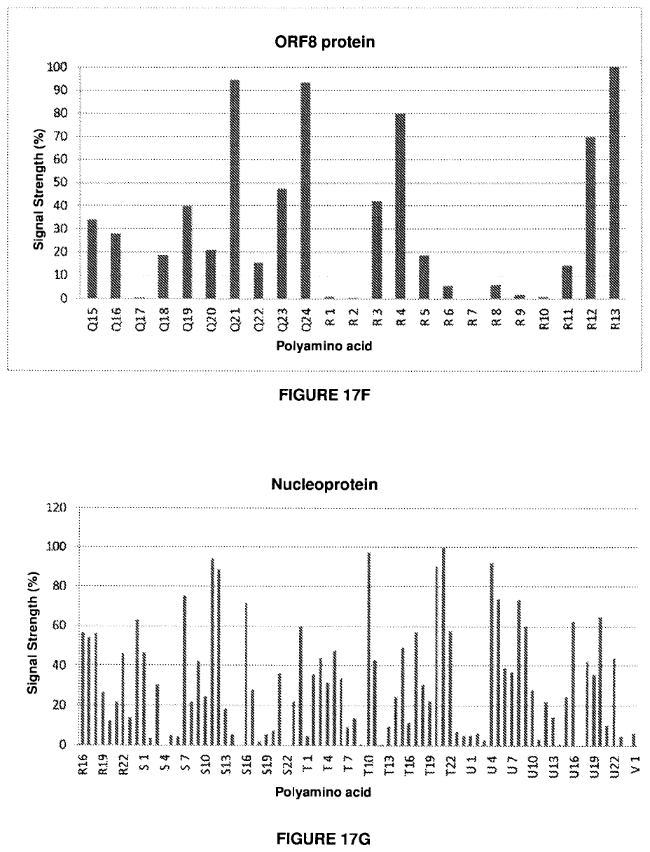

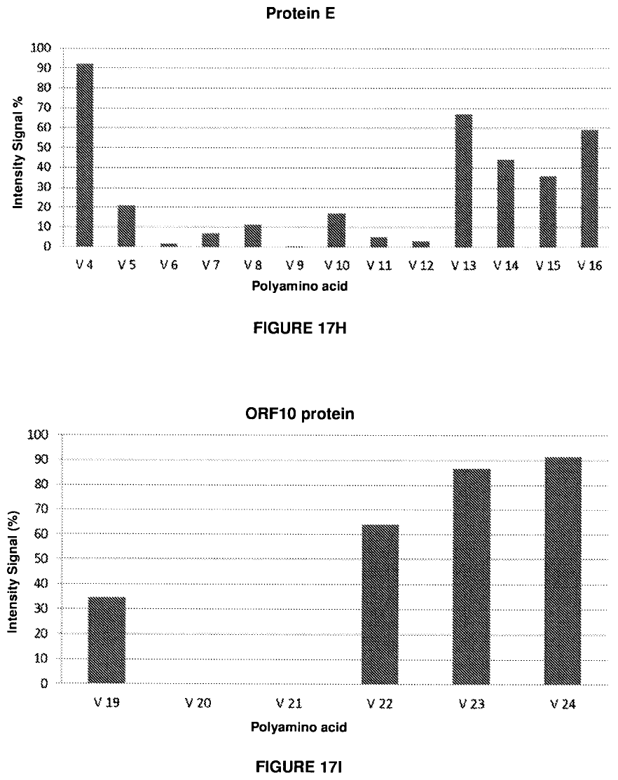

FIGS. 17A to 17I show the signal quantification of the results of the membrane spots from incubation with human sera revealed with goat anti-human IgG secondary antibody.

The human sera were significantly reactive to the polyamino acids of different viral proteins, as can be seen in FIGS. 17A to 171, when revealed by human anti-human IgA antibody, demonstrating that a large number of different polyamino acids have great potential for the diagnosis of the disease by means of this class of antibody predominantly present in mucous membranes.

### Example 26—Enzyme-Linked Immunosorbent Assay ELISA for Detection of Anti-SARS-CoV-2 Antibodies

Enzyme-linked immunosorbent assay (ELISA) was used to screen for anti-SARS-CoV2 antibody in patient sera. ELISA was performed by coating 96-well polystyrene plates with 1 μg/well of branched polyamino acids. For comparison of the results, the experiments of each group were performed in parallel and at the same time. To reduce the possible variation in the difference in performance between the tests, the reactivity index (RI) was employed, which was defined as the target's O.D.450 subtracted from the cutoff O.D.450. The primary human sera were diluted 100× in PBS/BSA 1% and the secondary antibodies goat anti-human IgG (Merck-Sigma), biotin-labeled goat anti-human IgG (Merck-Sigma) 8000×, followed by incubation with HRP-labeled high-sensitivity neutravidin (Thermo Fisher Scientific). The anti-IgA response was revealed with alkaline phosphatase-labeled goat anti-human Ig (KPL). TMB (3,3′, 5,5′ tetramethylbenzidine) was used as the substrate (Thermo Fisher Scientific), and the immune response was defined as significantly elevated when the reactivity index (IR) was greater than 1.

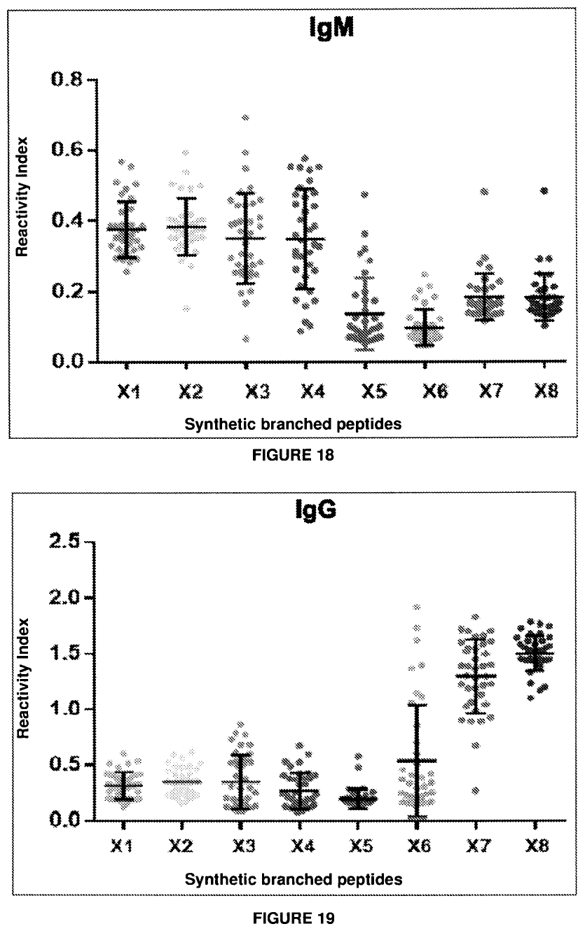

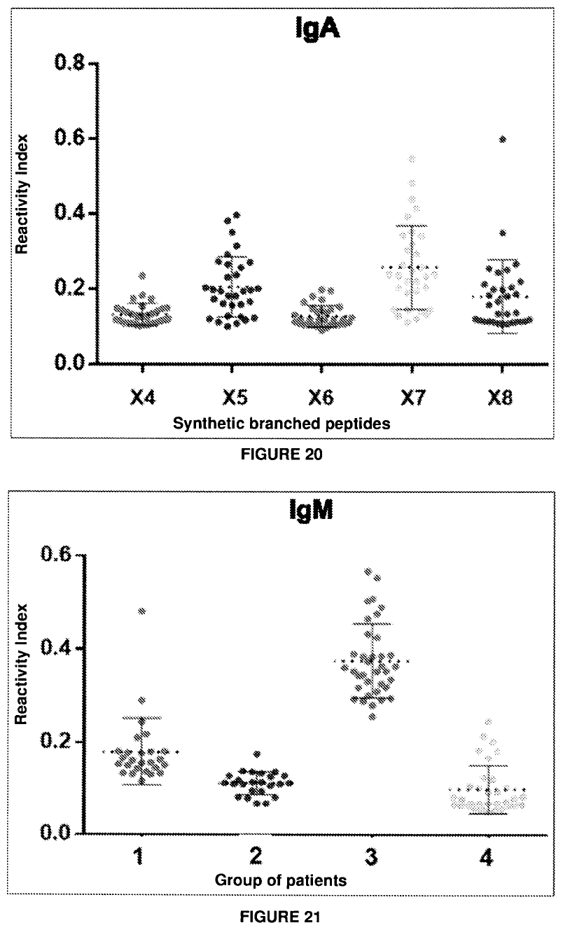

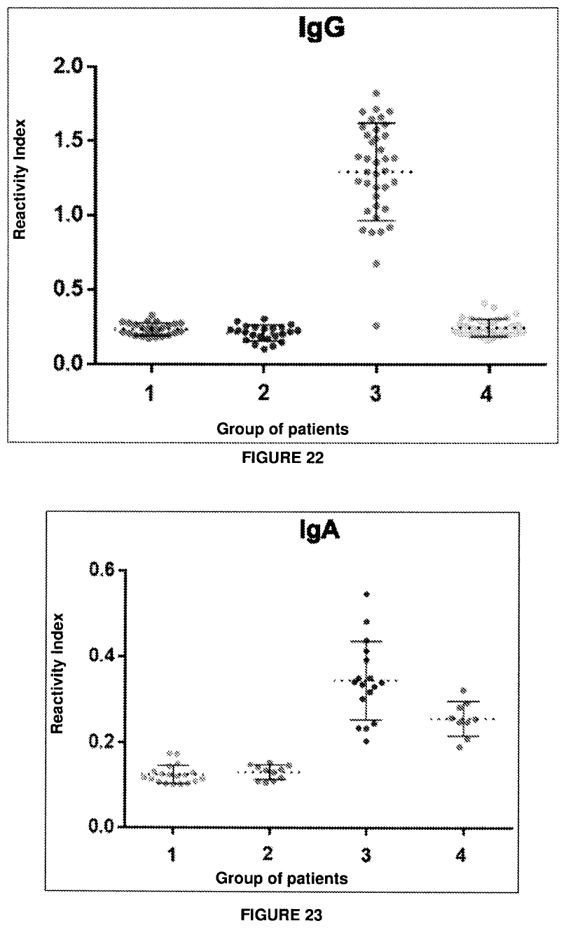

The results show that the branched polyamino acids SARS-X1 to SARS-X8 have differences in reactivity against anti-SARS-CoV2 antibodies and that these differences are related to the class of human antibody (IgM, IgG or IgA) detected and the status of the patient diagnosed with SARS-CoV2, as can be seen from the analysis of FIGS. 18 to 23. Observing such differences allows the design of diagnostic tests that may provide more accurate or robust information than simply a positive or negative diagnosis for anti-SARS-CoV2 antibodies. Thus, a diagnostic test can, in addition to detecting IgM, IgG, or IgA antibodies, also be designed to indicate whether individuals should be hospitalized even in the absence of symptoms.

### Example 27—Development of Receptacle Proteins for SARS-CoV-2

The “Tx” receptacle protein has been genetically manipulated to harbor SARS-CoV-2 epitopes. Reactive epitopes for sera from SARS-CoV-2 infected individuals were selected for construction of eight Tx proteins: Ag-COVID19, Ag COVID19 (H), Tx-SARS2-IgM, Tx-SARS2-IgG, Tx-SARS2-G/M, Tx-SARS2-IgA, Tx-SARS2-Universal and Tx-SARS-G5 (No RBD).

The genes corresponding to the Ag-COVID19, Ag COVID19 (H), Tx-SARS2-IgM, Tx-SARS2-IgG, Tx-SARS2-G/M, Tx-SARS2-IgA, Tx-SARS2-Universal, and Tx-SARS-G5 (No RBD) proteins, herein called, respectively, Ag-COVID19 gene, Ag-COVID19 (H) gene, Tx-SARS2-IgM gene, Tx-SARS2-IgG gene, Tx-SARS2-G/M gene, Tx-SARS2-IgA gene, Tx-SARS2-Universal gene, and Tx-SARS-G5 (No RBD) gene, are described in SEQ ID NO nucleotide sequences: 108 to SEQ ID NO:115. The amino acid sequences corresponding to Ag-COVID19, Ag COVID19 (H), Tx-SARS2-IgM, Tx-SARS2-IgG, Tx-SARS2-G/M, Tx-SARS2-IgA, Tx-SARS2-Universal and Tx-SARS-G5 (No RBD) proteins are described in SEQ ID NO 116 to 123, respectively.

From the SARS-CoV-2 epitope sequence mapping study, considering their diagnostic potential as clarified in Examples 19 to 26, the eight proteins described above harbored polyamino acids as shown in Tables 22 to 28.

TABLE 22
Ag-COVID19 and Ag COVID19 Proteins (H)

| Polyamino | Position in | Original | | |
|---|---|---|---|---|
| acid sequences | the protein | epitope protein | Sequence | SEQ ID no. |
| SARS-COV-2 | 1ª | S | FERDISTEIYQAGST | 124 |
| SARS-COV-2 | 4 | S | GSTPCNGVEGFNCYF | 125 |
| SARS-COV-2 | 8 | S | NSNNLDSKVGGNYNY | 126 |
| SARS-COV-2 | 10 | S | FERDISTEIYQAGST | 124 |
| SARS-COV-2 | 11 | S | GSTPCNGVEGFNCYF | 125 |
| SARS-COV-2 | 12 | S | YFPLQSYGFQPTNGV | 100 |
| SARS-COV-2 | 13ª | S | YFPLQSYGFQPTNGV | 100 |
| SARS-COV-2 | 13b | S | NSNNLDSKVGGNYNY | 126 |

TABLE 23
Tx-SARS2-IgM

| Polyamino | Position in | Original | | |
|---|---|---|---|---|
| acid sequences | the protein | epitope protein | Sequence | SEQ ID no. |
| SARS-COV-2 | 1ª | ORF3a | GSSGVVNPVM | 127 |
| SARS-COV-2 | 2 | N | NAPRITFGGPSDSTGS | 128 |
| SARS-COV-2 | 4 | ORF3a | GSSGVVNPVM | 127 |
| SARS-COV-2 | 5 | ORF8 | YIRVGARKSAPLIEL | 129 |
| SARS-COV-2 | 6 | S | SLIDLQELGKYEQYI | 130 |
| SARS-COV-2 | 8 | S | PFQQFGRDIADTTDA | 131 |
| SARS-COV-2 | 10 | M | MWLSYFIASFRL | 132 |
| SARS-COV-2 | 11 | S | RSYTPGDSSSGWTA | 101 |
| SARS-COV-2 | 12 | ORF3a | IVDEP | 133 |
| SARS-COV-2 | 13a | S | GFSALEPLVDLP | 134 |
| SARS-COV-2 | 13b | N | KTFPPTEPKKDKK | 135 |

TABLE 24
Tx-SARS2-IgG

| Polyamino | Position in | Original | | |
|---|---|---|---|---|
| acid sequences | the protein | epitope protein | Sequence | SEQ ID no. |
| SARS-COV-2 | 1a | S | LGVYHKNNKSWMESEFRVY | 136 |
| SARS-COV-2 | 3 | S | FNCYFPLQSYGFQPT | 137 |
| SARS-COV-2 | 4 | S | PLQSYGFQPT | 138 |
| SARS-COV-2 | 5 | N | AGNGGDAALALLLLD | 139 |
| SARS-COV-2 | 6 | S | RSYLTPGDSSS | 140 |
| SARS-COV-2 | 8 | S | ADQLTPTWRV | 141 |
| SARS-COV-2 | 10 | ORF3 | FIYNKIVDEP | 142 |
| SARS-COV-2 | 11 | ORF3 | KNPLLYDANY | 143 |
| SARS-COV-2 | 12 | N | RPQGLPNNTAS | 144 |
| SARS-COV-2 | 13a | N | LAEILQKNLIRQGTDYKHWPQIA | 145 |
| SARS-COV-2 | 13b | S | GKIADYNYKL | 146 |

TABLE 25
Tx-SARS2-G/M

| Polyamino | Position in | Original | | |
|---|---|---|---|---|
| acid sequences | the protein | epitope protein | Sequence | SEQ ID no. |
| SARS-COV-2 | 1a | S | FNCYFPLQSYGFQPT | 137 |
| SARS-COV-2 | 2 | N | RPQGLPNNTAS | 144 |
| SARS-COV-2 | 3 | ORF3 | FIYNKIVDEP | 142 |
| SARS-COV-2 | 4 | S | ADQLTPTWRV | 141 |
| SARS-COV-2 | 5 | S | GKIADYNYKL | 146 |
| SARS-COV-2 | 8 | ORF8 | YIRVGARKSAPLIEL | 129 |
| SARS-COV-2 | 9 | ORF3a | IVDEP | 133 |
| SARS-COV-2 | 10 | ORF3 | KNPLLYDANY | 143 |
| SARS-COV-2 | 11 | N | KTFPPTEPKKDKK | 135 |
| SARS-COV-2 | 12 | S | RSYLTPGDSSS | 140 |
| SARS-COV-2 | 13a | ORF3a | GSSGVVNPVMEPIYD | 147 |
| SARS-COV-2 | 13b | S | APGQTGKIADYNYKL | 148 |

TABLE 26
Tx-SARS2-IgA

| Polyamino | Position in | Original | | |
|---|---|---|---|---|
| acid sequences | the protein | epitope protein | Sequence | SEQ ID no. |
| SARS-COV-2 | 1a | N | ALPQRQKKQQTVTLL | 149 |
| SARS-COV-2 | 4 | ORF8 | GSKSPIQYID | 150 |
| SARS-COV-2 | 5 | ORF8 | DFLEYHDVRVVLDF | 151 |
| SARS-COV-2 | 6 | S | GINASVVNIQ | 152 |
| SARS-COV-2 | 7 | N | QFAPSASAFF | 153 |
| SARS-COV-2 | 8 | E | MYSFVSEETGTLIVN | 103 |
| SARS-COV-2 | 11 | N | PSGTWLTYTG | 154 |
| SARS-COV-2 | 12 | N | QFAPSASAFF | 153 |
| SARS-COV-2 | 13a | S | ELDKY | 155 |
| SARS-COV-2 | 13b | E | MYSFVSEETGTLIVN | 103 |

TABLE 27
Tx-SARS2-Universal

| Polyamino | Position in | Original | | |
|---|---|---|---|---|
| acid sequences | the protein | epitope protein | Sequence | SEQ ID no. |
| SARS-COV-2 | 1a | S | PLQSYGFQPTNGVGY | 104 |
| SARS-COV-2 | 4 | S | GIYQTSNFRV | 156 |
| SARS-COV-2 | 5 | N | KAYNVTQAFGRRGPE | 157 |
| SARS-COV-2 | 7 | S | GTNTSNQVAV | 158 |
| SARS-COV-2 | 8 | S | NPVLPFNDGVYFAST | 159 |
| SARS-COV-2 | 10 | S | YNYKLPDDFT | 160 |
| SARS-COV-2 | 11 | ORF6 | MFHLVDFQVTIAEIL | 161 |
| SARS-COV-2 | 12 | N | MKDLSPRWYF | 162 |
| SARS-COV-2 | 13a | N | DAALALLLLD | 163 |
| SARS-COV-2 | 13b | N | MKDLSPRWYF | 162 |

TABLE 28
Tx-SARS2-G5

| Polyamino | Position in | Original | | |
|---|---|---|---|---|
| acid sequences | the protein | epitope protein | Sequence | SEQ ID no. |
| SARS-COV-2 | 1a | S | LGVYHKNNKSWMESEFRVY | 136 |
| SARS-COV-2 | 3 | ORF3 | FIYNKIVDEP | 142 |
| SARS-COV-2 | 4 | ORF3 | KNPLLYDANY | 143 |
| SARS-COV-2 | 5 | N | AGNGGDAALALLLLD | 139 |
| SARS-COV-2 | 6 | S | RSYLTPGDSSS | 140 |
| SARS-COV-2 | 8 | S | ADQLTPTWRV | 141 |
| SARS-COV-2 | 10 | ORF3 | FIYNKIVDEP | 142 |
| SARS-COV-2 | 11 | ORF3 | KNPLLYDANY | 143 |
| SARS-COV-2 | 12 | N | RPQGLPNNTAS | 144 |
| SARS-COV-2 | 13a | N | LIRQGTDYKHWPQIA | 147 |
| SARS-COV-2 | 13b | S | GKIADYNYKL | 146 |

### Example 28 Expression of Receptacle Proteins with Polyamino Acids from SARS-CoV-2

The Ag-COVID19, Ag-COVID19 (H), Tx-SARS2-IgM, Tx-SARS2-IgG, Tx-SARS2-G/M, Tx-SARS2-IgA, Tx-SARS2-Universal and Tx-SARS-G5 (No RBD) proteins were expressed using pET24 plasmids harboring genes encoding each protein using restriction sites for the BamHI and XhoI enzymes. Each plasmid containing the gene for a specific protein was transferred to E. coli BL21 strains in order to promote the expression of the eight different proteins listed above.

The strain was grown overnight in LB medium and subsequently reseeded in the same medium, added kanamycin (30 μg/ml), on a shaker at 200 rpm, until it reached an optical turbidity density of 0.6-0.8 (600 nm). The BL21 strain expresses T7 RNA polymerase when induced by isopropyl β-D-1-thiogalactopyranoside (IPTG). Then IPTG (q.s.p. 1 mM) is added to the culture and the same culture conditions are maintained for another 3 h at 37° C.

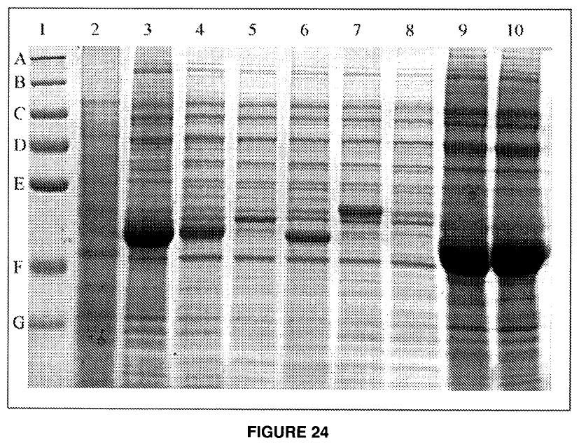

The culture of each bacterial strain was subjected to centrifugation and the pellet resuspended in 10% CelLytic™ (Sigma, BR) in 150 mM NaCl and 50 mM Tris, pH 8.0. Aliquots of the recombinant proteins (1 μg/well) were subjected to SDS-containing polyacrylamide gel electrophoresis (SDS-PAGE) (Laemmli, Nature 227: 680-685, 1970). Concentration gels (stacking gel) and separation gels (running gel) were prepared at an acrylamide concentration of 4% and 11%, respectively (table 5, below). Samples were prepared under denaturing conditions in 62.5 mM Tris-HCl buffer, pH 6.8, 2% SDS, 5% β-mercaptoethanol, 10% glycerol and boiled at 95° C. for 5 min (Hames B D, Gel electrophoresis of proteins: a practical approach. 3. Ed. Oxford. 1998). After electrophoresis, the proteins were detected by staining with comassie blue Simply Blue R250 (ThermoFisher, BR). The marker PageRuler Plus Prestained Standards was used as a molecular weight reference (ThermoFisher, BR). FIG. 24, shows the band of Ag-COVID19 (column 3), Ag-COVID19 (H) (column 4), Tx-SARS2-IgM (column 5), Tx-SARS2-IgG (column 6), Tx-SARS2-G/M (column 7), Tx-SARS2-IgA (column 8), Tx-SARS2-Universal (column 9) and Tx-SARS-G5 (No RBD) (column 10). Column 1 shows the molecular weight marker: A) 250 kDa; B) 130 kDa; C) 100 kDa; D) 70 kDa; E) 55 kDa; F) 35 kDa and G) 25 kDa. Column 2 shows a total extract of uninduced bacteria.

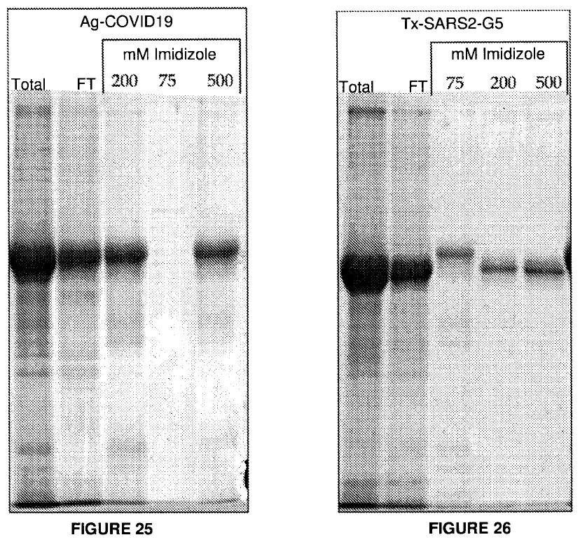

Alternatively, the culture was also subjected to centrifugation and the pellet resuspended in urea buffer (100 mM NaH2PO4, 10 mM Tris-base, 8 M urea, pH 8.0). The solution was subjected to chromatography by a nickel affinity column (HisTrap) mL per minute, previously equilibrated in buffer A (50 mM Tris-HCl, pH 8.0, 100 mM NaCl and 5 mM imidazole). After binding, the resin was washed with 10 mL of buffer A. The protein was eluted in steps of buffer B (50 mM Tris-HCl, pH 8.0, 100 mM NaCl) with 75 mM, 200 mM, and 500 mM imidazole at a flow rate of 0.7 mL/min for 45 minutes. FIG. 25 shows the pattern corresponding to the Ag-COVID19 protein with the six histidine tail indicating the 200 mM eluted concentration. FIG. 26 shows the pattern corresponding to the affinity-purified SARS2-G5 protein by using 200 mM imidazole (the elution performed with 75 mM shows a contaminant).

The results show that the Tx receptacle protein can be used to create new and different proteins with great ease. Additionally, the expression of different receptacle proteins harboring different polyamino acids can be performed using the same expression protocol, generating great savings in inputs, time, and infrastructure. The inclusion of a six-histidine tail was shown to be a potential facilitator for purification at high purity levels.

### Example 29—Enzyme-Linked Immunosorbent Assay (ELISA) for Detecting Anti-SARS-CoV-2 Antibody Using Ag-COVID19 and SARS2-G5 Proteins

Enzyme-linked immunosorbent assay (ELISA) was used to screen for the presence of anti-SARS-CoV-2 antibody. The performance of Ag-COVID19 and SARS2-G5 proteins was evaluated against a panel of sera from individuals affected by SARS-CoV-2 virus infections.

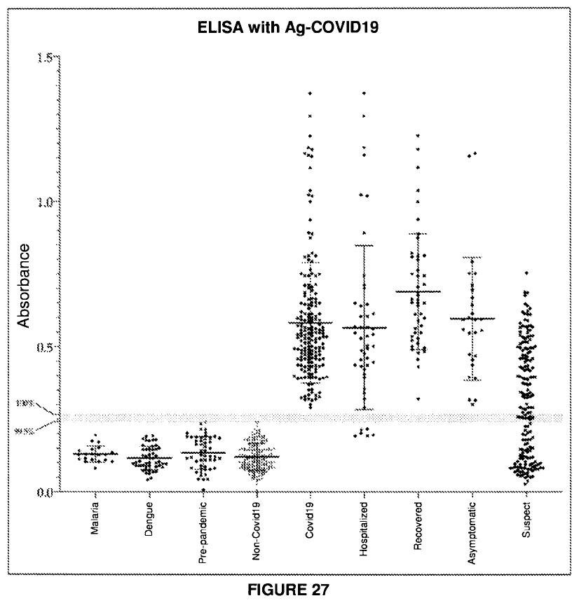

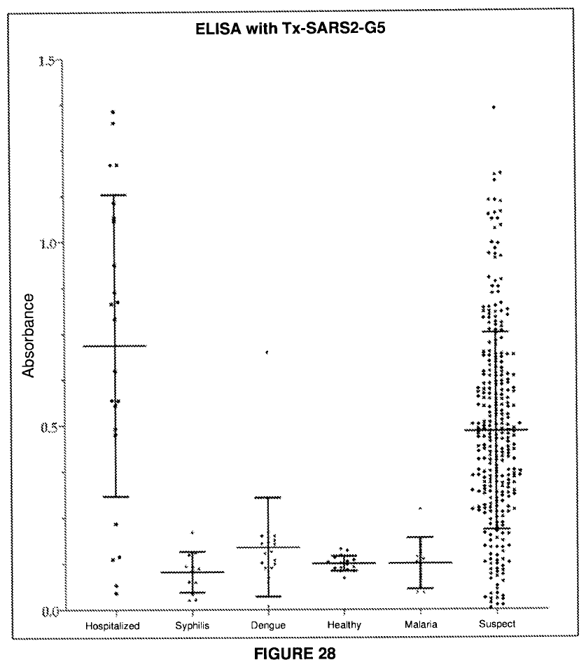

ELISA was performed by coating 96-well polystyrene plates with 1 μg/well in solution (0.3 M Urea, pH8.0) of Ag-COVID19 protein (FIG. 27) or Tx-SARS2-G5 protein (FIG. 28) at 4° C. for 12-18 h. The wells were washed with saline-phosphate buffer (PBS) solution added Tween 20 (PBS-T, 10 mM sodium phosphate—Na3PO4, 150 mM sodium chloride—NaCl and 0.05% Tween-20, pH 7.4) and then incubated with 1×PBS buffer containing 5% (weight/volume) dehydrated skim milk for 2 h at 37° C.

Then, the wells were washed three times with PBS-T buffer and incubated with human serum samples diluted 1:100 in PBS/BSA 1% for 1 h at 37° C. After the incubation period, the wells were washed three times with PBS-T and then incubated with biotin-labeled goat anti-human IgG antibody (Merck-Sigma) at a dilution of 1:8000 for 1 h at 37° C. Subsequently, HRP-labeled high-sensitivity neutravidin (Thermo Fisher Scientific) was added. The wells were washed again three times with PBS-T buffer and TMB substrate (3.3′, 5.5′ tetramethylbenzidine, Thermo Fisher Scientific) was used. After 30 minutes and under shelter from light, the absorbance was measured in an ELISA plate reader at 405 nm.

The results show that Ag-COVID19 protein (FIG. 27) and Tx-SARS2-G5 protein (FIG. 28) have proven useful for detecting antibodies against SARS-CoV-2 by demonstrating excellent sensitivity and specificity indices. As can be seen from FIGS. 27 and 28, the proteins harboring polyamino acids from SARS-CoV-2 did not detect antibodies in sera from individuals collected prior to the SARS-CoV-2 pandemic, healthy or affected by diseases such as dengue, malaria, and syphilis. Differently, the proteins detected antibodies in sera from individuals diagnosed positive for SARS-CoV-2 (symptomatic or asymptomatic), hospitalized or already recovered patients.

### Example 30—Ag-COVID19 Protein as Vaccine Composition

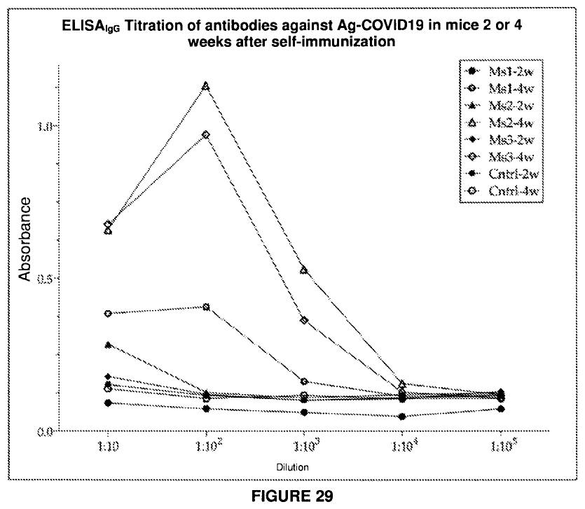

The Ag-COVID19 protein was produced and purified according to the protocols described in this patent application. Three mice were subjected to inoculation with 10 μg of Ag-COVID19 protein in 25 μl of PBS suspended in Freud's complete adjuvant (25 μl) on days 0, 14, 21 and 28. The negative control was performed using an animal inoculated with PBS. Blood samples from the animals were collected before each reinoculation and submitted to ELISA. Plasma was separated from the collected blood by centrifugation and subjected to serial dilution to perform antibody measurement (FIG. 29). The results showed excellent antibody production against Ag-COVID19 after four weeks from the first injection.

### Example 31—Use of Ag-COVID19 Protein for Purification of Anti SARS-CoV-2 Antibodies

Purification of anti-SARS-CoV-2 antibodies from sera of patients diagnosed with COVID19 was performed using the antibody affinity principle. Ag-COVID19 protein was conjugated to Sepharose™ 4B activated with CNBr (GE Healthcare, USA). A 10 mL serum sample from a SARS-CoV2 positive patient was diluted in 10 mL PBS and subjected to Sepharose-Ag-COVID19 for 1 hour. The mixture was then placed on a chromatography column. After the solution had passed through the column, 10 mL of PBS was added to the chromatographic system and then 5 mL of a 100 mM sodium citrate buffer at pH 4. Fractions of 0.5 ml were collected sequentially as they were recovered from the column and quantified for the presence of antibodies by spectrophotometry at 280 nm. The absorbance of each fraction was converted to protein concentration and plotted as a function of the volume of the fraction.

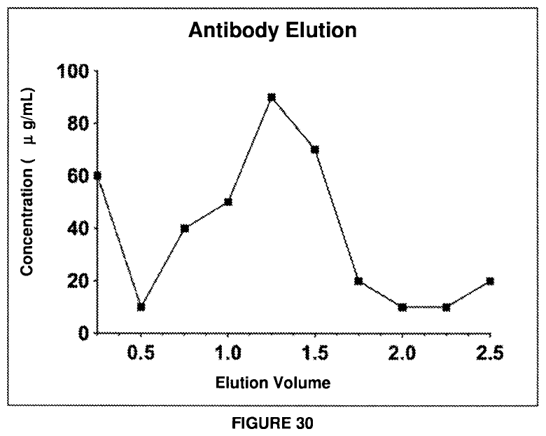

The results demonstrate that the Ag-COVID19 protein can be useful as an input for affinity purification of antibodies from patients previously infected with SARS-CoV-2 (FIG. 30), indicating its importance in generating usable inputs for passive immunization to address the COVID-19 pandemic.

1. A protein receptacle comprising a stable protein structure that supports, at different sites, the insertion of four or more exogenous polyamino acid sequences simultaneously.

2. The protein receptacle according to claim 1, further comprising an amino acid sequence at least 90% identical to SEQ ID NO: 1.

3. The protein receptacle according to claim 1, further comprising an amino acid sequence at least 90% identical to SEQ ID NO: 3.

4. The protein receptacle according to claim 1, further comprising an amino acid sequence at least 90% identical to SEQ ID NO: 77.

5. The protein receptacle according to claim 1, further comprising insertion sites for exogenous polyamino acid sequences in protein loops facing the external medium.

6. The protein receptacle according to claim 1, wherein the insertion of the exogenous polyamino acid sequences simultaneously does not interfere with the production conditions of the receptacle protein.

7. The protein receptacle according to claim 2, wherein it contains exogenous polyamino acid sequences simultaneously for use in vaccine compositions, in diagnostics, or in the development of laboratory reagents.

8. The protein receptacle according to claim 2, wherein the exogenous polyamino acid sequences do not lose their immunogenic characteristics upon simultaneous insertion into the protein loops of the protein receptacle.

9. The protein receptacle according to claim 2, further comprising the exogenous polyamino acid sequences SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 and SEQ ID NO:16, simultaneously.

10. The protein receptacle according to claim 9, further comprising the amino acid sequence shown in SEQ ID NO: 18.

11. The protein receptacle according to claim 3, further comprising the exogenous polyamino acid sequences SEQ ID NO:21, SEQ ID NO:22, SEQ ID NO:23, SEQ ID NO:24, SEQ ID NO:25, SEQ ID NO:26, SEQ ID NO:27, SEQ ID NO:28 and SEQ ID NO:29, simultaneously.

12. The protein receptacle according to claim 11, further comprising the amino acid sequence shown in SEQ ID NO. 20.

13. The protein receptacle according to claim 3, further comprising multiple copies of the exogenous polyamino acid sequence simultaneously, SEQ ID NO:30.

14. The protein receptacle according to claim 13, further comprising the amino acid sequence shown in SEQ ID NO. 31.

15. The protein receptacle according to claim 3, further comprising the exogenous polyamino acid sequences SEQ ID NO:35, SEQ ID NO:36, SEQ ID NO:37, SEQ ID NO:38, SEQ ID NO:39 and SEQ ID NO:40, simultaneously.

16. The protein receptacle according to claim 15, further comprising the amino acid sequence shown in SEQ ID NO. 33.

17. The protein receptacle according to claim 3, further comprising the exogenous polyamino acid sequences SEQ ID NO: 41, SEQ ID NO: 42, SEQ ID NO: 43 and SEQ ID NO: 44, simultaneously.

18. The protein receptacle according to claim 17, further comprising the amino acid sequence shown in SEQ ID NO: 45.

19. The protein receptacle according to claim 3, further comprising the exogenous polyamino acid sequences SEQ ID NO: 47, SEQ ID NO: 48, SEQ ID NO: 49 and SEQ ID NO: 50, simultaneously.

20. The protein receptacle according to claim 19, further comprising the amino acid sequence shown in SEQ ID NO: 51.

21. The protein receptacle according to claim 3, further comprising the exogenous polyamino acid sequences SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 95 and SEQ ID NO: 96, simultaneously.

22. The protein receptacle according to claim 21, further comprising the amino acid sequence shown in SEQ ID NO: 64.

23. The protein receptacle according to claim 3, further comprising the exogenous polyamino acid sequences SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74 and SEQ ID NO: 97, simultaneously.

24. The protein receptacle according to claim 23, further comprising the amino acid sequence shown in SEQ ID NO: 75.

25. The protein receptacle according to claim 4, further comprising the exogenous polyamino acid sequences simultaneously SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87 and SEQ ID NO: 98.

26. The protein receptacle according to claim 25, further comprising the amino acid sequence shown in SEQ ID NO: 88.

27. The protein receptacle according to claim 4, further comprising the exogenous polyamino acid sequences SEQ ID NO: 7, SEQ ID NO: 9, SEQ ID NO: 10, SEQ ID NO: 12, SEQ ID NO: 14, SEQ ID NO: 15, SEQ ID NO: 16, SEQ ID NO: 92, SEQ ID NO: 93, SEQ ID NO: 94 and SEQ ID NO: 99, simultaneously.

28. The protein receptacle according to claim 27, further comprising the amino acid sequence shown in SEQ ID NO: 90.

29. The protein receptacle according to claim 4, further comprising exogenous polyamino acid sequences as defined in SEQ ID NO: 100, 124, 125 and 126, simultaneously; or SEQ ID NO:101, 127, 128, 129, 130, 131, 132, 133, 134 and 135, simultaneously; or SEQ ID NO: 136, 137, 138, 139, 140, 141, 142, simultaneously; or SEQ ID NO: 129, 133, 135, 137, 140, 141, 142, 143, 144, 146, 147, and 148, simultaneously; or SEQ ID NO: 103, 149, 150, 151, 152, 153, 154, 155, simultaneously; or SEQ ID NO: 104, 156, 157, 158, 159, 160, 161, 162, and 163, simultaneously; or SEQ ID NO: 136, 139, 140, 141, 142, 143, 144, 146 and 147, simultaneously.

30. The protein receptacle according to claim 29, further comprising any of the amino acid sequences shown in SEQ ID NO: 116-123.

31. A polynucleotide comprising any one of SEQ ID NO: 2, 4, 78, 17, 19, 32, 34, 46, 52, 63, 76, 89, 91, 108-115 and their degenerate sequences, capable of generating, respectively, the polypeptides defined by SEQ ID NO: 1, 3, 77, 18, 20, 31, 33, 45, 51, 64, 75, 88, 90, 116-123.

32. A vector comprising the polynucleotide as defined in claim 31.

33. An expression cassette comprising polynucleotide as defined in claim 31.

34. A cell comprising the vector as defined in claim 32.

35. A method for producing the protein receptacle wherein it introduces into competent cells of interest the polynucleotide as defined in claim 31; performing culture of the competent cells and performing isolation of the receptacle protein containing the exogenous polyamino acids of choice.

36. The method for producing the protein receptacle according to claim 35, wherein it is free from interference by the insertion of various exogenous polyamino acid sequences.

37. A method of pathogen identification or in vitro disease diagnosis wherein it uses the receptacle protein as defined in claim 1.

38. The method of pathogen identification or in vitro disease diagnosis according to claim 37, wherein it promotes the diagnosis of Chagas disease, rabies, pertussis, yellow fever, Oropouche virus infections, Mayaro virus infections, IgE hypersensitivity, D. pteronyssinus allergy, or COVID-19.

39. A method of using the protein receptacle as defined in claim 1, characterized in that it is a laboratory reagent.

40. A method of using the protein receptacle as defined in claim 1, wherein it is for the production of a vaccine composition for immunization against Chagas disease, rabies, pertussis, yellow fever, infections by the Oropouche, Mayaro viruses-, hypersensitivity to IgE, allergy to D. pteronyssinus or COVID19.

41. A diagnostic kit comprising the protein receptacle as defined in claim 1.

42. A cell comprising the expression cassette as defined in claim 33.


SEQUENCE LISTING (163 sequences)

SEQ ID NO: 1 - protein, 246 aa, Artificial Sequence
  Aequorea victoria
MVASKGEELFTGVVPILIELDGDVNGHKFFVRGEGEGDATIGKLSLKFICTTGKLPVPWPTLVTTLTYGVQCFSRYPDHMKRHDFFKSAMPEGYVQERTIYFKDVDGTYKTRAEVKFEGTDTLVNRIVLKGIDFKEDGNILKGHKLEYNFNSHNVYITADKQKNGIKANFTVRHNVEDGSVQLADHYQQNTPIGDGPVLLPDNHYLSTQTILLKDPNELKRDHMVLLEYVTAAGITLGMDELYKEF

SEQ ID NO: 2 - DNA, 744 bp, Aequorea victoria
catatggtggctagcaaaggtgaagaactgttcaccggtgttgttccgatcctgattgaactggacggtgacgttaacggtcacaaattctttgttcgtggtgaaggtgaaggtgacgcgaccatcggtaaactgagtctgaaattcatctgcaccaccggtaagcttccggttccgtggccgaccctggttaccaccctgacctacggtgttcagtgcttctctcgttacccggaccacatgaaacgtcacgacttcttcaaatctgcgatgccggaaggttacgttcaggaacgtaccatctacttcaaagacgtcgacggtacttacaaaacccgtgcggaagttaaattcgaaggtaccgacaccctggttaaccgtatcgttctgaaaggcattgacttcaaagaagacggtaacatccttaagggtcacaaactggaatacaacttcaactctcacaacgtttacatcaccgcggacaaacagaaaaacggtatcaaagcgaacttcaccgttcgtcacaacgttgaagacggatccgttcagctggcggaccactaccagcagaacaccccgatcggtgacggtccggttctgctgccggacaaccactacctgtctacccagaccattctgctgaaagacccgaacgagctcaaacgtgaccacatggttctgctggaatatgttacggcggcgggtatcaccctgggtatggacgaactgtacaaagaattctaa

SEQ ID NO: 3 - protein, 246 aa, Artificial Sequence
  Aequorea victoria
MVASKGEELFTGVVPILVELDGDVNGHKFSVRGEGEGDATIGKLTLKFICTTGKLPVPWPTLVTTLTYGVQCFARYPDHMKRHDFFKSAMPEGYVQERTISFKDVDGKYKTRAVVKFEGTDTLVNRIVLKGTDFKEDGNILKGHKLEYNFNSHNVYITADKQKNGIKANFTVRHNVEDGGVQLADHYQQNTPIGDGPVLLPDNHYLSTQTVLSKDPNELKRDHMVLLEFVTAAGITLGMDELYKEF

SEQ ID NO: 4 - DNA, 753 bp, Artificial Sequence
  Aequorea victoria
catatggtggctagcaagggcgaggagctgttcaccggcgtcgtccccatcctggtcgagctggacggcgacgttaacggccacaagttctccgtccggggcgagggcgagggcgacgccaccatcggcaagctgaccctgaagttcatctgcaccaccggcaagcttcccgtcccctggcccaccctggtcaccaccctgacctatggcgtccagtgcttcgcccggtatcccgaccacatgaagcggcacgacttcttcaagtccgccatgcccgagggctatgtccaggagcggaccatctccttcaaggacgtcgatggcaagtataagacccgggccgtcgtcaagttcgagggtaccgacaccctggtcaaccggatcgtcctgaagggcaccgacttcaaggaggacggcaacatccttaagggccacaagctggagtataacttcaactcccacaacgtctatatcaccgcggacaaacagaagaacggcatcaaggccaacttcaccgtccggcacaacgtcgaggacggatccgtccagctggccgaccactatcagcagaacacccccatcggcgacggtccggtcctgctgcccgacaaccactatctgtccacccagaccgtcctgtccaaggacccgaacgagctcaaacgggaccacatggtcctgctggagttcgtcaccgccgccggcatcaccctgggcatggacgagctgtataaggaattctaatgactcgag

SEQ ID NO: 5 - DNA, 15 bp, Artificial Sequence
  SYNTHETIC CONSTRUCT
catatggtggctagc

SEQ ID NO: 6 - DNA, 18 bp, Artificial Sequence
  SYNTHETIC CONSTRUCT
gaattctaatgactcgag

SEQ ID NO: 7 - protein, 16 aa, Trypanosoma cruzi
KFAELLEQQKNAQFPG

SEQ ID NO: 8 - protein, 7 aa, Trypanosoma cruzi
KAAAAPA

SEQ ID NO: 9 - protein, 7 aa, Trypanosoma cruzi
KAAIAPA

SEQ ID NO: 10 - protein, 15 aa, Trypanosoma cruzi
GDKPSPFGQAAAADK

SEQ ID NO: 11 - protein, 9 aa, Trypanosoma cruzi
KQKAAEATK

SEQ ID NO: 12 - protein, 10 aa, Trypanosoma cruzi
AEPKPAEPKS

SEQ ID NO: 13 - protein, 10 aa, Trypanosoma cruzi
AEPKSAEPKP

SEQ ID NO: 14 - protein, 15 aa, Trypanosoma cruzi
GTSEEGSRGGSSMPS

SEQ ID NO: 15 - protein, 11 aa, Trypanosoma cruzi
SPFGQAAAGDK

SEQ ID NO: 16 - protein, 9 aa, Trypanosoma cruzi
KQRAAEATK

SEQ ID NO: 17 - DNA, 1128 bp, Artificial Sequence
  Aequorea victoria, Trypanosoma cruzi
atgaaattcgcggaactgctggaacagcagaaaaacgcgcagttcccgggtaaagctagcaaaggtgaagaactgttcaccggtgttgttccgatcctgattgaactggacggtgacgttaacggtcacaaattctttgttcgtggtgaaggtgaaggtgacgcgaccatcggtaaactgagtctgaaattcatctgcaccaccggtaagcttaaagcggcggcggcgccggcgaagcttccggttccgtggccgaccctggttaccaccctgacctacggtgttcagtgcttctctcgttacccggaccacatgaaacgtcacgacttcttcaaatctgcgatgccggaaggttacgttcaggaacgtaccatctacttcaaagacgtcaaagcggcgatcgcgccggcggacgtcgacggtacttacaaaacccgtgcggaagttaaattcgaaggtaccggtgacaaaccgtctccgttcggtcaggcggcggcggcggacaaaggtaccgacaccctggttaaccgtatcgttctgaaaggcattgacttcaaagaagacggtaacatccttaagcagaaagcggcggaagcgaccaaacttaagggtcacaaactggaatacaacttcaactctcacaacgtttacatcaccgcggacgcggaaccgaaaccggcggaaccgaaatctaccgcggacaaacagaaaaacggtatcaaagcgaacttcaccgttcgtcacaacgttgaagacggatccgctgaaccgaaatctgcggaaccgaaaccgggatccgttcagctggcggaccactaccagcagaacaccccgatcggtgacggtccgggcacctctgaagaaggttctcgtggtggttcttctatgccgtctgacggtccggttctgctgccggacaaccactacctgtctacccagaccattctgctgaaagacccgaacgagctctctccgttcggtcaggcggcggcgggtgacaaagagctcaaacgtgaccacatggttctgctggaatatgttacggcggcgggtatcaccctgggtatggacgaactgtacaaagaattcaaacagcgtgcggcggaagcgaccaaatgatgactcgag

SEQ ID NO: 18 - protein, 372 aa, Artificial Sequence
  Aequorea victoria, Trypanosoma cruzi
MKFAELLEQQKNAQFPGKASKGEELFTGVVPILIELDGDVNGHKFFVRGEGEGDATIGKLSLKFICTTGKLKAAAAPAKLPVPWPTLVTTLTYGVQCFSRYPDHMKRHDFFKSAMPEGYVQERTIYFKDVKAAIAPADVDGTYKTRAEVKFEGTGDKPSPFGQAAAADKGTDTLVNRIVLKGIDFKEDGNILKQKAAEATKLKGHKLEYNFNSHNVYITADAEPKPAEPKSTADKQKNGIKANFTVRHNVEDGSAEPKSAEPKPGSVQLADHYQQNTPIGDGPGTSEEGSRGGSSMPSDGPVLLPDNHYLSTQTILLKDPNELSPFGQAAAGDKELKRDHMVLLEYVTAAGITLGMDELYKEFKQRAAEATK

SEQ ID NO: 19 - DNA, 1029 bp, Artificial Sequence
  Aequorea victoria, Lyssavirus Rabies lyssavirus
atgctgcatgattttcgttccgatgctagcaaaggtgaagaactgtttaccggtgttgttccgattctggttgaactggatggtgatgttaatggccacaaattttcagttcgtggtgaaggcgaaggtgatgcaaccattggtaaactgaccctgaaatttatctgtaccaccggtaagcttccagttccgtggccgaccctggttaccaccctgacctatggtgttcagtgttttgcacgttatccggatcacatgaaacgccacgattttttcaaaagcgcaatgccggaaggttatgttcaagaacgtaccattagctttaaagacgtctgcaaactgaaactgtgtggtgttctgggtctggacgtcgatggtaaatacaaaacccgtgcagttgtgaaatttgagggtacctgtaaactgaaactgtgcggagttagcggtctgggtaccgataccctggtgaatcgtattgttctgaaaggcaccgattttaaagaagatggcaacattcttaagtgggttgcaatgcagaccagcaatcttaagggtcataaactggaatacaacttcaacagccacaacgtgtatattaccgcggatctgcacgattttcatagcgataccgcggacaaacagaaaaatggtattaaagccaattttaccgtgcggcataatgttgaagatggatcctgcaaactgaaactgtgcggtgtgcctggtctgggatccgttcagctggcagatcattatcagcagaataccccgattggtgacggtccggttgatgaacgtggtctgtataaagacggtccggtgctgctgccggataatcattatctgagcacccagacagttctgagcaaagatccgaatgagctcctgcatgatctgcatagtgatgagctcaaacgtgatcacatggttctgctggaatttgttaccgcagcaggtattaccctgggtatggatgaactgtacaaagaattcaaaagtgtgcgcacctggaacgaaatctaactcgag

SEQ ID NO: 20 - protein, 340 aa, Artificial Sequence
  Aequorea victoria, Lyssavirus Rabies lyssavirus
MLHDFRSDASKGEELFTGVVPILVELDGDVNGHKFSVRGEGEGDATIGKLTLKFICTTGKLPVPWPTLVTTLTYGVQCFARYPDHMKRHDFFKSAMPEGYVQERTISFKDVCKLKLCGVLGLDVDGKYKTRAVVKFEGTCKLKLCGVSGLGTDTLVNRIVLKGTDFKEDGNILKWVAMQTSNLKGHKLEYNFNSHNVYITADLHDFHSDTADKQKNGIKANFTVRHNVEDGSCKLKLCGVPGLGSVQLADHYQQNTPIGDGPVDERGLYKDGPVLLPDNHYLSTQTVLSKDPNELLHDLHSDELKRDHMVLLEFVTAAGITLGMDELYKEFKSVRTWNEI

SEQ ID NO: 21 - protein, 11 aa, Rabies virus
CKLKLCGVLGL

SEQ ID NO: 22 - protein, 11 aa, Rabies virus
CKLKLCGCSGL

SEQ ID NO: 23 - protein, 11 aa, Rabies virus
CKLKLCGVPGL

SEQ ID NO: 24 - protein, 8 aa, Rabies virus
VDERGLYK

SEQ ID NO: 25 - protein, 8 aa, Rabies virus
WVAMQTSN

SEQ ID NO: 26 - protein, 9 aa, Rabies virus
KSVRTWNEI

SEQ ID NO: 27 - protein, 7 aa, Rabies virus
LHDFHSD

SEQ ID NO: 28 - protein, 7 aa, Rabies virus
LHDFRSD

SEQ ID NO: 29 - protein, 7 aa, Rabies virus
LHDLHSD

SEQ ID NO: 30 - protein, 13 aa, Artificial Sequence
  Equus ferus caballus
DVLFTWYVDGTEV

SEQ ID NO: 31 - protein, 344 aa, Artificial Sequence
  Aequorea victoria, Equus ferus caballus
MDVLFTWYVDGTEVASKGEELFTGVVPILVELDGDVNGHKFSVRGEGEGDATIGKLTLKFICTTGKLPVPWPTLVTTLTYGVQCFARYPDHMKRHDFFKSAMPEGYVQERTISFKDVDGKYKTRAVVKFEGTDTLVNRIVLKGTDFKEDGNILKDVLFTWYVDGTEVLKGHKLEYNFNSHNVYITADVLFTWYVDGTEVGGSGTADKQKNGIKANFTVRHNVEDGSVQLADHYQQNTPIGDGPGGSGDVLFTWYVDGTEVDGPVLLPDNHYLSTQTVLSKDPNELDVLFTWYVDGTEVGGSGELKRDHMVLLEFVTAAGITLGMDELYKEFDVLFTWYVDGTEV

SEQ ID NO: 32 - DNA, 1035 bp, Artificial Sequence
  Aequorea victoria, Equus ferus caballus
atggatgttctgtttacctggtatgttgatggcaccgaagtggctagcaaaggtgaagaactgtttaccggtgttgttccgattctggttgaactggatggtgatgttaatggccacaaattttcagttcgtggtgaaggcgaaggtgatgcaaccattggtaaactgaccctgaaatttatctgtaccaccggtaagcttccagttccgtggccgaccctggttaccaccctgacctatggtgttcagtgttttgcacgttatccggatcacatgaaacgccacgattttttcaaaagcgcaatgccggaaggttatgttcaagaacgtaccattagcttcaaagacgtcgatggtaaatacaaaacccgtgccgttgttaaatttgaaggtaccgataccctggtgaatcgtattgttctgaaaggcaccgattttaaagaagatggcaacattcttaaggacgtgctgttcacatggtatgtggacggtacagaagttcttaagggtcacaaactggaatacaactttaacagccacaacgtgtatattaccgcggacgtactgtttacgtggtacgtagacggaacggaagttggtggtagcggcaccgcggacaaacagaaaaatggtattaaagccaattttaccgtgcggcataatgttgaagatggatccgttcagctggcagatcattatcagcagaataccccgattggtgacggtccgggtggttcaggtgatgtcctgttcacttggtacgtcgatggaaccgaggtggacggtccggttctgctgccggataatcattatctgagcacccagaccgttctgagcaaagatccgaatgagctcgatgtactgttcacgtggtatgtggatgggactgaagtgggtggtagtggtgagctcaaacgtgatcacatggtgctgctggaatttgttaccgcagcaggtattaccctgggtatggatgaactgtataaagaattcgatgtgctgtttacttggtacgtggacgggactgaggtttaa

SEQ ID NO: 33 - protein, 380 aa, Artificial Sequence
  Aequorea victoria, Orthobunyavirus Oropouche orthobunyavirus
MYIEKDDSDALKALFASKGEELFTGVVPILVELDGDVNGHKFSVRGEGEGDATIGKLTLKFICTTGKLPVPWPTLVTTLTYGVQCFARYPDHMKRHDFFKSAMPEGYVQERTISFKDVGNFMVLSVDDVDGKYKTRAVVKFEGTGNFMVLSVDDGTDTLVNRIVLKGTDFKEDGNILKTSRPMVDLTFGGVQLKGHKLEYNFNSHNVYITADKTSRPMVDLTFGGVQTADKQKNGIKANFTVRHNVEDGSIFNDVPQRTTSTFDPGSVQLADHYQQNTPIGDGPIFNDVPQRTTSTFDPDGPVLLPDNHYLSTQTVLSKDPNELYSDLFSKNLVTEYELKRDHMVLLEFVTAAGITLGMDELYKEFYIEKDDSDALKALF

SEQ ID NO: 34 - DNA, 1149 bp, Artificial Sequence
  Aequorea victoria, Orthobunyavirus Oropouche orthobunyavirus
atgtacatcgagaaagacgatagcgacgcactgaaagcactgttcgctagcaaaggtgaagaactgtttaccggtgttgttccgattctggttgaactggatggtgatgttaatggccacaaattttcagttcgtggtgaaggcgaaggtgatgcaaccattggtaaactgaccctgaaatttatctgtaccaccggtaagcttccagttccgtggccgaccctggttaccaccctgacctatggtgttcagtgttttgcacgttatccggatcacatgaaacgccacgattttttcaaaagcgcaatgccggaaggttatgttcaagaacgtaccattagctttaaagacgtcggcaattttatggttctgagcgttgatgacgtcgacggtaaatacaaaacccgtgcagttgttaaatttgaaggtaccggtaactttatggtgctgtcagttgatgatggtaccgataccctggtgaatcgtattgttctgaaaggcaccgattttaaagaagatggcaacattcttaagaccagccgtccgatggttgatctgacctttggtggtgtgcagcttaagggtcataaactggaatataatttcaacagccacaacgtgtatatcaccgcggacaaaacctcacgtcctatggtggacctgacattcggtggcgttcagaccgcggacaaacagaaaaatggtattaaagccaattttaccgtgcggcataatgttgaggacggatccatttttaacgatgttccgcagcgtaccaccagtacctttgatccgggatccgttcagctggcagatcattatcagcagaataccccgattggtgacggtccgatctttaatgatgtgcctcagcgcacaacctcaaccttcgatccggacggtccggttctgctgccggataatcattatctgagcacccagaccgttctgagcaaagatccgaatgagctctatagcgacctgtttagcaaaaatctggttaccgaatatgagctcaaacgtgatcacatggtgctgctggaatttgttaccgcagcaggtattaccctgggtatggatgaactgtataaagaattctatatcgaaaaagatgattccgatgccctgaaagccctgttttaactcgag

SEQ ID NO: 35 - protein, 14 aa, Orthobunyavirus Oropouche orthobunyavirus
YIEKDDSDALKALF

SEQ ID NO: 36 - protein, 10 aa, Orthobunyavirus Oropouche orthobunyavirus
GNFMVLSVDD

SEQ ID NO: 37 - protein, 15 aa, Orthobunyavirus Oropouche orthobunyavirus
KTSRPMVDLTFGGVQ

SEQ ID NO: 38 - protein, 15 aa, Orthobunyavirus Oropouche orthobunyavirus
IFNDVPQRTTSTFDP

SEQ ID NO: 39 - protein, 14 aa, Orthobunyavirus Oropouche orthobunyavirus
LYSDLFSKNLVTEY

SEQ ID NO: 40 - protein, 14 aa, Orthobunyavirus Oropouche orthobunyavirus
YIEKDDSDALKALF

SEQ ID NO: 41 - protein, 10 aa, Mayaro virus
KLSATDWSAI

SEQ ID NO: 42 - protein, 8 aa, Mayaro virus
KPKPQPEK

SEQ ID NO: 43 - protein, 9 aa, Mayaro virus
KKMTPSDQI

SEQ ID NO: 44 - protein, 10 aa, Mayaro virus
VELPWPLETI

SEQ ID NO: 45 - protein, 340 aa, Artificial Sequence
  Aequorea victoria, Alphavirus mayaro virus
MKLSATDWSAIKGEELFTGVVPILVELDGDVNGHKFSVRGEGEGDATIGKLTLKFICTTGKLPVPWPTLVTTLTYGVQCFARYPDHMKRHDFFKSAMPEGYVQERTISFKDVKPKPQPEKDVDGKYKTRAVVKFEGTKKMTPSDQIGTDTLVNRIVLKGTDFKEDGNILKVELPWPLETILKGHKLEYNFNSHNVYITADKPKPQPEKTADKQKNGIKANFTVRHNVEDGSKLSATDWSAIGSVQLADHYQQNTPIGDGPVELPWPLETIDGPVLLPDNHYLSTQTVLSKDPNELKKMTPSDQIELKRDHMVLLEFVTAAGITLGMDELYKLSATDWSAI

SEQ ID NO: 46 - DNA, 1023 bp, Artificial Sequence
  Aequorea victoria, Alphavirus mayaro virus
atgaaactgagcgcaaccgattggagcgcaattaaaggtgaagaactgtttaccggtgttgttccgattctggttgaactggatggtgatgttaatggccacaaattttcagttcgtggtgaaggcgaaggtgatgcaaccattggtaaactgaccctgaaatttatctgtaccaccggtaagcttccagttccgtggccgaccctggttaccaccctgacctatggtgttcagtgttttgcacgttatccggatcacatgaaacgccacgattttttcaaaagcgcaatgccggaaggttatgttcaagaacgtaccattagcttcaaagacgtcaaaccgaaaccgcagccggaaaaagacgtcgatggtaaatacaaaacccgtgccgttgttaaatttgaaggtaccaaaaaaatgaccccgagcgatcagattggtaccgataccctggtgaatcgtattgttctgaaaggcaccgattttaaagaagatggcaacatccttaaggtggaactgccgtggcctctggaaaccattcttaagggtcataaactggaatataatttcaacagccacaacgtgtatatcaccgcggacaaacctaaacctcaacctgaaaaaaccgcggacaaacagaaaaatggcatcaaagcaaattttaccgtgcgccataatgttgaggatggatccaaactgtcagccaccgattggtcagcaattggatccgttcagctggcagatcattatcagcagaataccccgattggtgacggtccggttgagctgccttggccactggaaacaattgacggtccggtgctgctgccggataatcattatctgagcacccagaccgttctgagcaaagatccgaatgagctcaaaaaaatgacaccgtcagatcagatcgagctcaaacgtgatcacatggttctgctggaatttgttaccgcagcaggtattaccctgggtatggatgaactgtataaactgagtgcgacagactggtctgcaatctaa

SEQ ID NO: 47 - protein, 9 aa, Mayaro virus
HRIRLLLQS

SEQ ID NO: 48 - protein, 9 aa, Mayaro virus
SYRTGAERV

SEQ ID NO: 49 - protein, 9 aa, Mayaro virus
NGVKQTVDV

SEQ ID NO: 50 - protein, 9 aa, Mayaro virus
QSRTLDSRD

SEQ ID NO: 51 - protein, 338 aa, Artificial Sequence
  Aequorea victoria, Alphavirus mayaro virus
MHRIRLLLQSKGEELFTGVVPILVELDGDVNGHKFSVRGEGEGDATIGKLTLKFICTTGKLPVPWPTLVTTLTYGVQCFARYPDHMKRHDFFKSAMPEGYVQERTISFKDVSYRTGAERVDVDGKYKTRAVVKFEGTNGVKQTVDVGTDTLVNRIVLKGTDFKEDGNILKQSRTLDSRDLKGHKLEYNFNSHNVYITADQSRTLDSRDTADKQKNGIKANFTVRHNVEDGSHRIRLLLQSGSVQLADHYQQNTPIGDGPSYRTGAERVDGPVLLPDNHYLSTQTVLSKDPNELNGVKQTVDVELKRDHMVLLEFVTAAGITLGMDELYKHRIRLLLQS

SEQ ID NO: 52 - DNA, 1017 bp, Artificial Sequence
  Aequorea victoria, Alphavirus mayaro virus
atgcatcgtattcgtctgctgctgcagagcaaaggtgaagaactgtttaccggtgttgttccgattctggttgaactggatggtgatgttaatggccacaaattttcagttcgtggtgaaggcgaaggtgatgcaaccattggtaaactgaccctgaaatttatctgtaccaccggtaagcttccagttccgtggccgaccctggttaccaccctgacctatggtgttcagtgttttgcacgttatccggatcacatgaaacgccacgattttttcaaaagcgcaatgccggaaggttatgttcaagaacgtaccattagctttaaagacgtcagctatcgtaccggtgcagaacgtgttgacgtcgatggtaaatacaaaacccgtgccgttgttaaatttgaaggtaccaatggcgttaaacagaccgttgatgttggtaccgataccctggtgaatcgtattgttctgaaaggcaccgattttaaagaagatggcaacattcttaagcagagccgtaccctggatagccgtgatcttaagggtcataaactggaatataatttcaacagccacaacgtgtatattaccgcggaccagtcacgcaccctggattcacgtgacaccgcggacaaacagaaaaatggtattaaagccaattttaccgtgcggcataatgttgaagatggatcccatcgtatccgcctgctgctgcaaagcggatccgttcagctggcagatcattatcagcagaataccccgattggtgacggtccgagttatcgcacaggtgccgaacgcgtggacggtccggttctgctgccggataatcattatctgagcacccagaccgttctgagcaaagatccgaatgagctcaatggtgtgaaacaaacagtggatgtagagctcaaacgtgatcacatggtgctgctggaatttgttaccgcagcaggtattaccctgggtatggatgaactgtataaacaccgcattcggctgctgctgcaatcataa

SEQ ID NO: 53 - protein, 14 aa, Bordetella pertussis
PYTSRRSVASIVGT

SEQ ID NO: 54 - protein, 10 aa, Bordetella pertussis
QYYDYEDATF

SEQ ID NO: 55 - protein, 10 aa, Bordetella pertussis
GPKQLTFEGK

SEQ ID NO: 56 - protein, 10 aa, Bordetella pertussis
DATFETYALT

SEQ ID NO: 57 - protein, 9 aa, Bordetella pertussis
LTVEDSPYP

SEQ ID NO: 58 - protein, 9 aa, Bordetella pertussis
ALATYQSEY

SEQ ID NO: 59 - protein, 14 aa, Bordetella pertussis
PGIVIPPKALFTQQ

SEQ ID NO: 60 - protein, 9 aa, Bordetella pertussis
AVEAERAGR

SEQ ID NO: 61 - protein, 9 aa, Bordetella pertussis
TTTEYSNAR

SEQ ID NO: 62 - protein, 14 aa, Bordetella pertussis
ERAGEAMVLVYYES

SEQ ID NO: 63 - DNA, 1101 bp, Artificial Sequence
  Aequorea victoria, Bordetella pertussis
atgccctacacctcacgtcgcagcgtagcttcgattgttggcacgtcttattggaaagggtctcaatactatgactatgaagatgcaacttttaaaggagaggagttgtttaccggcgtggtgccgatccttgtggagttggatggagacgttaacggtcacaagttttcagttcgcggtgaaggcgaaggcgacgcgactattggtaagcttaccttgaagttcatctgtactacggggcctaagcagcttacgttcgaggggaaattacctgtcccctggccaacattggtcacgacattaacctatggcgtgcagtgttttgcgcgttaccccgatcacatgaagcgtcacgacttctttaagtccgcgatgccggaagggtatgtccaggaacgcacgatttcgttcaaagacgcgacctttgagacttacgcattaaccgggggctcgggcgaggcagccgcaaaagaagctgctgctgacggcaaatacaaaactcgtgcggtcgtaaaattcgagggagacactttagttaatcgtattgtgctgaaagggactgacttcaaggaagatggaaacattttgacggttgaggatagcccctatcctggccataaacttgagtacaacttcaattcacataatgtctacattacagcacttgcaacgtatcagtctgagtacgataagcagaagaatggtatcaaggctaattttacggtccgtcataatgttgaggatgggagcgtgcagttagctgaccattatcaacaaaatacgcccattggggaccccggcatcgtgattcccccaaaagcattattcacccagcaagtgcttttaccagacaaccattacttgagcacccaaacggtgttaagcaaggaccccaatgagaaagcagtggaggcggaacgcgctggacgtgatcatatggtacttcttgaattcgtaacggcggccgggatcactttgggaatggacgagttatataaagagcgtgcgggtgaggccatggtgcttgtgtattacgagtcagaagccgcgaaggaggctgccaagacaacaacggaatacagtaacgcccgtaaaaagtaa

SEQ ID NO: 64 - protein, 366 aa, Artificial Sequence
  Aequorea victoria, Bordetella pertussis
MPYTSRRSVASIVGTSYWKGSQYYDYEDATFKGEELFTGVVPILVELDGDVNGHKFSVRGEGEGDATIGKLTLKFICTTGPKQLTFEGKLPVPWPTLVTTLTYGVQCFARYPDHMKRHDFFKSAMPEGYVQERTISFKDATFETYALTGGSGEAAAKEAAADGKYKTRAVVKFEGDTLVNRIVLKGTDFKEDGNILTVEDSPYPGHKLEYNFNSHNVYITALATYQSEYDKQKNGIKANFTVRHNVEDGSVQLADHYQQNTPIGDPGIVIPPKALFTQQVLLPDNHYLSTQTVLSKDPNEKAVEAERAGRDHMVLLEFVTAAGITLGMDELYKERAGEAMVLVYYESEAAKEAAKTTTEYSNARKK

SEQ ID NO: 65 - protein, 14 aa, Yellow fever virus
SPWSWPDLDLKPGA

SEQ ID NO: 66 - protein, 14 aa, Yellow fever virus
DGNCDGRGKSTRST

SEQ ID NO: 67 - protein, 14 aa, Yellow fever virus
VFSPGRKNGSFIID

SEQ ID NO: 68 - protein, 14 aa, Yellow fever virus
HVQDCDESVLTRLE

SEQ ID NO: 69 - protein, 14 aa, Yellow fever virus
DCDGSILGAAVNGK

SEQ ID NO: 70 - protein, 9 aa, Yellow fever virus
FTTRVYMDA

SEQ ID NO: 71 - protein, 14 aa, Yellow fever virus
RDSDDWLNKYSYYP

SEQ ID NO: 72 - protein, 14 aa, Yellow fever virus
ESEMFMPRSIGGPV

SEQ ID NO: 73 - protein, 14 aa, Yellow fever virus
AEAEMVIHHQHVQD

SEQ ID NO: 74 - protein, 19 aa, Yellow fever virus
LEHEMWRSRADEINAIFEE

SEQ ID NO: 75 - protein, 430 aa, Yellow fever virus
MGSSHHHHHHSSGLVPRGSHMSPWSWPDLDLKPGAKGEELFTGVVPILVELDGDVNGHKFSVRGEGEGDATIGKLTLKFICTTGKLPVPWPTLVTTLTYGVQCFARYPDHMKRHDFFKSAMPEGYVQERTISFKDGNCDGRGKSTRSTGGSGEAAAKEAAAKKYKTRAVVKFEGVFSPGRKNGSFIIDGGSGEAAAKEAAAKDTLVNRIVLKGTDFKEDGNILGHKHVQDCDESVLTRLEYNFNSHNVYITADCDGSILGAAVNGKQKNGIKANFTVRHNVEDFTTRVYMDAGTSWKGGSVQLADHYQQNTPIGDRDSDDWLNKYSYYPVLLPDNHYLSTQTVLSKDPNESEMFMPRSIGGPVKRDHMVLLEFVTAAGITLGMDELYKLEHEMWRSRADEINAIFEETSYWKGSAEAEMVIHHQHVQDKK

SEQ ID NO: 76 - DNA, 1293 bp, Artificial Sequence
  Flavivrus yellow fever virus
atgggcagcagccatcatcatcatcatcacagcagcggcctggtgccgcgcggcagccatatgtccccatggagttggcctgaccttgatttaaagcccggtgctaaaggagaagaactgttcacaggggtcgttcccatcttagtggagctggacggcgacgtgaacggccataaattcagtgtgcgcggggaaggagaaggggacgcaacgattggtaagttgacgctgaaatttatctgtacaactggtaagcttccagtgccgtggccgacgcttgtgacgacacttacttatggagtccagtgttttgcgcgttatccagatcacatgaaacgccacgacttctttaagtccgcaatgccggagggctacgtgcaggaacgcacaatctcgtttaaggacgggaactgcgatggccgtgggaaaagtacacgctcaacgggcggatcaggcgaagccgcagcaaaagaggctgcggccaaaaaatataaaactcgtgccgtagtgaaatttgagggagtgttttccccgggccgtaagaacggcagttttatcattgacggcggctcaggagaagcggctgcaaaggaggccgccgcgaaggacaccttagtgaaccgcattgttttgaaaggaacggattttaaagaggacggaaacattttaggacacaagcatgtccaagactgcgatgagagtgttcttacgcgtttagaatataatttcaatagtcataacgtatacattacggctgattgtgatggcagtatcttaggagcggctgttaacgggaagcaaaagaatggaatcaaagccaactttactgttcgtcataatgtcgaagacttcaccactcgtgtctacatggatgccggtactagctggaaaggcgggtccgttcagcttgcggatcattaccaacaaaacactccgatcggggaccgtgactccgatgattggctgaacaagtattcatactaccctgtgctgctgcccgataaccattatttgagcacacagaccgtgctgtctaaagatcctaatgaatcagaaatgtttatgcctcgttcgatcgggggacccgttaaacgcgatcacatggtattgttggaattcgttacggcagcgggcatcacgcttggtatggatgagttgtataaacttgagcatgaaatgtggcgctcgcgtgcagacgaaatcaatgcaatttttgaagagactagctattggaagggtagtgcggaagctgaaatggtcattcaccaccagcatgtccaggacaaaaaataa

SEQ ID NO: 77 - protein, 245 aa, Artificial Sequence
  SYNTHETIC CONSTRUCT
MGAHASVIKPEMKIKLRMEGAVNGHKFVIEGEGIGKPYEGTQTLDLTVEEGAPLPFSYDILTPAFQYGNRAFTKYPEDIPDYFKQAFPEGYSWERSMTYEDQGICIATSDITMEGDCFFYEIRFDGTNFPPNGPVMQKKTLKWEPSTEKMYVEDGVLKGDVEMALLLEGGGHYRCDFKTTYKAKKDVRLPDAHEVDHRIEILSHDKDYNKVRLYEHAEARYSGGGSGGGASGKPIPNPLLGLDST

SEQ ID NO: 78 - DNA, 738 bp, Artificial Sequence
  SYNTHETIC CONSTRUCT
atgggtgcgcacgcgtctgttatcaaaccggaaatgaaaatcaaactgcgtatggaaggtgcggttaacggtcacaaattcgttatcgaaggtgaaggtatcggtaaaccgtacgaaggtacccagaccctggacctgaccgttgaagaaggtgcgccgctgccgttctcttacgacatcctgaccccggcgttccagtacggtaaccgtgcgttcaccaaatacccggaagacatcccggactacttcaaacaggcgttcccggaaggttactcttgggaacgttctatgacctacgaagaccagggtatctgcatcgcgacctctgacatcaccatggaaggtgactgcttcttctacgaaatccgtttcgacggtaccaacttcccgccgaacggtccggttatgcagaaaaaaaccctgaaatgggaaccgtctaccgaaaaaatgtacgttgaagacggtgttctgaaaggtgacgttgaaatggcgctgctgctggaaggtggtggtcactaccgttgcgacttcaaaaccacctacaaagcgaaaaaagacgttcgtctgccggacgcgcacgaagttgaccaccgtatcgaaatcctgtctcacgacaaagactacaacaaagttcgtctgtacgaacacgcggaagcgcgttactctggtggtggttctggtggtggtgcgtctggtaaaccgatcccgaacccgctgctgggtctggactctacctaa

SEQ ID NO: 79 - protein, 20 aa, Dermatophagoides pteronyssinus
DLRQMRTVTPIRMQGGCGSC

SEQ ID NO: 80 - protein, 20 aa, Dermatophagoides pteronyssinus
GCGSCWAFSGVAATESAYLA

SEQ ID NO: 81 - protein, 15 aa, Dermatophagoides pteronyssinus
QESYYRYVAREQSCR

SEQ ID NO: 82 - protein, 15 aa, Dermatophagoides pteronyssinus
HAVNIVGYSNAQGVD

SEQ ID NO: 83 - protein, 20 aa, Dermatophagoides pteronyssinus
CHGSEPCIIHRGKPFQLEAV

SEQ ID NO: 84 - protein, 20 aa, Dermatophagoides pteronyssinus
YDIKYTWNVPKIAPKSENVV

SEQ ID NO: 85 - protein, 15 aa, Dermatophagoides pteronyssinus
NTKTAKIEIKASIDG

SEQ ID NO: 86 - protein, 15 aa, Dermatophagoides pteronyssinus
GVLACAIATHAKIRD

SEQ ID NO: 87 - protein, 20 aa, Dermatophagoides pteronyssinus
PKDPHKFYICSNWEAVHKDC

SEQ ID NO: 88 - protein, 366 aa, Dermatophagoides pteronyssinus
MPKDPHKFYICSNWEAVHKDCSVIKPEMKIKLRMEGAVHAVNIVGYSNAQGVDHKFVIEGEGIGKPYEGTQTLDLTVEEGAPLPFSYDILTPAFQYGNRAFTKYPEDIPDYFKQAFPEGYSWERSMTYGVLACAIATHAKIRDGICIATSDITMEYDIKYTWNVPKIAPKSENVVCFFYEIRFDGTQESYYRYVAREQSCRTLKWEPSTEKMYVEDGVLKGDVEMALLLCHGSEPCIIHRGKPFQLEAVHYRCDFKTTYKAGCGSCWAFSGVAATESAYLAHEVDHRIEILSHDKDYNKVRLYEHAEARYSGGSGDLRQMRTVTPIRMQGGCGSCGGSGNTKTAKIEIKASIDGLDSTHHHHHHKK

SEQ ID NO: 89 - DNA, 1101 bp, Dermatophagoides pteronyssinus
atgccgaaagatccccacaagttctatatttgctcaaactgggaggccgtgcacaaagattgttctgttatcaagccagagatgaaaatcaaactgcgcatggagggggctgtccatgcggtaaacattgtgggttactcaaatgcgcagggagtggaccataagtttgtcatcgaaggcgagggcatcggtaaaccttatgaaggcactcagactttggatttaacggtcgaagaaggagcgcctttgccattttcgtatgatatcttaaccccggcgtttcaatacggaaatcgcgcgttcacaaagtatcccgaagacatccccgattacttcaagcaagcttttccagaaggctacagctgggagcgttcaatgacgtatggagtccttgcatgtgctatcgccacacatgctaaaatccgcgatggtatttgtattgccacgtcagatatcacaatggaatacgatatcaagtacacttggaacgtgccaaaaatcgctcccaagtcagaaaacgtggtgtgtttcttctatgagattcgttttgatggtacccaagagtcttattatcgttatgttgcacgtgaacaaagttgtcgtacgttgaagtgggagccttctacagaaaaaatgtatgtggaggacggcgtgcttaaaggagacgtggagatggcactgcttttgtgtcatgggtccgaaccgtgtattattcatcgcggaaaaccctttcaattggaagcggtccactaccgctgcgattttaagaccacatataaagctggatgcggttcctgctgggcgttctccggtgtagctgccactgaatcggcttacttggcgcacgaagtcgatcatcgtatcgagattttatcgcacgacaaggattacaacaaagtccgcttatatgagcacgcagaagcacgctactctggtggcagtggtgacttacgtcagatgcgtacagtgacacctattcgtatgcaaggaggatgtggtagctgtggaggatcaggcaacaccaagacggcgaaaattgaaattaaagcatccattgatggacttgattcgacgcaccaccaccatcatcacaaaaagtag

SEQ ID NO: 90 - protein, 333 aa, Trypanosoma cruzi
MGAHASVIKFAELLEQQKNAQFPGKPEMKIKLRMEGAVNGHKFVIEGEGIGKPYEGTQTLDLTVEEDSSAHSTPSTPAYDILTPAFQYGNRAFTKYPEDIPDYFKQAFPEGYSWERSMTYEDQGICIATSDITMEGDKPSPFGQAAAADKCFFYEIRFDGTFGQAAAGDKPSTLKWEPSTEKMYVEAEPKPAEPKSVLKGDVEMALLLTSSTPPSGTENKPATGHYRCDFKTTYKAGTSEEGSRGGSSMPSHEVDHRIEILSHSPFGQAAAGDKKVRLYEHAEARYSGGGSGKAAIAPAGGASGKQRAAEATKPIPNPLLGLDSTHHHHHHKK

SEQ ID NO: 91 - DNA, 1002 bp, Trypanosoma cruzi
atgggtgcgcacgcaagcgtgattaagtttgcggaacttttagaacagcagaaaaacgcacagttcccggggaaacccgaaatgaaaatcaaacttcgcatggagggggcggtaaacggccacaagtttgttatcgagggggaaggtatcggaaagccttacgagggaacgcagactttggacttaacagtagaggaagactcatctgcacattcaaccccgtctactccggcatatgatattttaactcctgcgtttcaatatgggaaccgtgcatttactaaatacccagaggacatccctgattatttcaaacaggcttttccggaaggttactcatgggagcgctctatgacatatgaagatcaaggaatctgtattgccacttcggacatcaccatggagggagacaagccgagcccatttggtcaagcagctgccgcagataaatgttttttctatgaaattcgtttcgacgggacatttggacaagcggctgcaggcgacaagcctagcacgttgaagtgggagccaagcaccgaaaagatgtacgttgaggctgaaccgaagccagcggagccgaaatcagtcttaaagggggatgttgaaatggctttgcttctgacgagcagcacgccaccaagtggcacagaaaacaaacccgccacaggacattatcgctgcgattttaagactacatataaggctggtacctctgaggaggggtctcgcggaggtagtagcatgccgtcacacgaggttgaccaccgcattgagatcttatctcactccccttttggtcaggctgcagctggggataagaaggtgcgtctttatgagcacgcggaggcccgttactctggtggaggcagtgggaaagcggcaattgcccccgcaggcggcgcgtcagggaaacaacgcgccgctgaggcgacgaaaccgatcccgaacccgctgttgggacttgacagtacccaccatcatcaccaccacaagaaatag

SEQ ID NO: 92 - protein, 12 aa, Trypanosoma cruzi
DSSAHSTPSTPA

SEQ ID NO: 93 - protein, 11 aa, Trypanosoma cruzi
FGQAAAGDKPS

SEQ ID NO: 94 - protein, 16 aa, Trypanosoma cruzi
TSSTPPSGTENKPATG

SEQ ID NO: 95 - protein, 6 aa, Artificial Sequence
  SYNTHETIC CONSTRUCT
SYWKGS

SEQ ID NO: 96 - protein, 8 aa, Artificial Sequence
  SYNTHETIC CONSTRUCT
EAAKEAAK

SEQ ID NO: 97 - protein, 7 aa, Artificial Sequence
  SYNTHETIC CONSTRUCT
TSYWKGS

SEQ ID NO: 98 - protein, 4 aa, Artificial Sequence
  SYNTHETIC CONSTRUCT
GGSG

SEQ ID NO: 99 - protein, 5 aa, Betacoronavirus SARS-CoV-2
GGASG

SEQ ID NO: 100 - protein, 15 aa, Betacoronavirus SARS-CoV-2
YFPLQSYGFQPTNGV

SEQ ID NO: 101 - protein, 15 aa, Betacoronavirus SARS-CoV-2
RSYTPGDSSSGWTAG

SEQ ID NO: 102 - protein, 15 aa, Betacoronavirus SARS-CoV-2
GKTFPPTEPKKDKKG

SEQ ID NO: 103 - protein, 15 aa, Betacoronavirus SARS-CoV-2
MYSFVSEETGTLIVN

SEQ ID NO: 104 - protein, 15 aa, Betacoronavirus SARS-CoV-2
PLQSYGFQPTNGVGY

SEQ ID NO: 105 - protein, 15 aa, Betacoronavirus SARS-CoV-2
GGMKDLSPRWYFGGG

SEQ ID NO: 106 - protein, 15 aa, Betacoronavirus SARS-CoV-2
GSKSPIQYIDGGGGG

SEQ ID NO: 107 - protein, 15 aa, Betacoronavirus SARS-CoV-2
YIRGARKSAPLIELG

SEQ ID NO: 108 - DNA, 1041 bp, Betacoronavirus SARS-CoV-2
atggttaattatctcggagcacacgctttcgagcgcgacatctcaacggaaatttatcaagccggttcaaccatgaagatcaagctccgtatggaaggggcggtcaacggtcacaaattcgtcatcgaaggggaaggcattggcaagccatacgaagggacacagactttggacctcactgtggaagaaggttcatctggtgaagcggcgaaggaagcggccaagggaagcacaccatgcaacggcgtggaaggtttcaattgctacttttacgacatccttaccccggcgttccaatatggtaaccgtgccttcacgaagtacccagaagatatccctgattactttaagcaggcatttcctgaaggttattcgtgggagcgcagtatgacctatgaggaccaaggtatttgtatcgcgacgagcgacattaccatggaaggggactgtttcttctacgagattcgcttcgatggaactaattcgaataacctggacagtaaagttggtggcaactacaactataccttaaaatgggagccaagtacagaaaagatgtacgttgaagacggggtgttgaagggtgacgtggaaatggcattactcttgtttgagcgcgacatttcaaccgagatttaccaagccgggtcgacaggcgggtccggaacaagctactggaaggggagtcactatcgctgtgattttaagacgacctacaaagctggtagcactccatgtaacggagtagaggggttcaactgctactttcatgaagtagatcaccgtattgagattttatcacactactttcctctgcaatcgtatggcttccaacccacgaatggcgttggtagtagcggcgaagccgccaaggaagcagccaagaaggtgcgcctgtacgagcacgccgaggcgtactttcctctccagtcctatggattccaacctaccaatggagttggcggctcgggcggcggtgcttccgggaattcgaacaacttagatagcaaggtgggtgggaactataattatctcgagtag

SEQ ID NO: 109 - DNA, 1059 bp, Betacoronavirus SARS-CoV-2
atggttaattatctcggagcacacgctttcgagcgcgacatctcaacggaaatttatcaagccggttcaaccatgaagatcaagctccgtatggaaggggcggtcaacggtcacaaattcgtcatcgaaggggaaggcattggcaagccatacgaagggacacagactttggacctcactgtggaagaaggttcatctggtgaagcggcgaaggaagcggccaagggaagcacaccatgcaacggcgtggaaggtttcaattgctacttttacgacatccttaccccggcgttccaatatggtaaccgtgccttcacgaagtacccagaagatatccctgattactttaagcaggcatttcctgaaggttattcgtgggagcgcagtatgacctatgaggaccaaggtatttgtatcgcgacgagcgacattaccatggaaggggactgtttcttctacgagattcgcttcgatggaactaattcgaataacctggacagtaaagttggtggcaactacaactataccttaaaatgggagccaagtacagaaaagatgtacgttgaagacggggtgttgaagggtgacgtggaaatggcattactcttgtttgagcgcgacatttcaaccgagatttaccaagccgggtcgacaggcgggtccggaacaagctactggaaggggagtcactatcgctgtgattttaagacgacctacaaagctggtagcactccatgtaacggagtagaggggttcaactgctactttcatgaagtagatcaccgtattgagattttatcacactactttcctctgcaatcgtatggcttccaacccacgaatggcgttggtagtagcggcgaagccgccaaggaagcagccaagaaggtgcgcctgtacgagcacgccgaggcgtactttcctctccagtcctatggattccaacctaccaatggagttggcggctcgggcggcggtgcttccgggaattcgaacaacttagatagcaaggtgggtgggaactataattatctcgagcaccaccaccaccaccactga

SEQ ID NO: 110 - DNA, 1128 bp, Betacoronavirus SARS-CoV-2
atggtaaattatatagggtcaagtggagttgtcaacccggtgatgggtggctcgggtggtccagcgccagcgccgatgaaaattaagttgcgtatggaaggggcggttaatgctccgcgtatcaccttcggcggtcctagcgatagcaccggcggtggttctggcgaagccgcgaagacgagctactggaagggttcgcataaattcgtgattgagggtgagggcattggtaaaccgtatgagggtacgcagaccttagatctgaccgttgaggaaggttctagcggcgtcgttaacccggttatgtatgatattttgaccccggcgttccaatatggaaaccgcgcattcaccaaatacccggaatatattcgtgttggtgcccgtaaaagcgctccgctgatcgagctgggttattcctgggagcgctccatgacctacggctccctgatcgacctgcaagagttgggtaagtacgagcaatacatcgaggcagccgctaaagaagccgcagcgaagggcggcgcttctggcatctgcattgcgaccagcgacatcaccatggagggcgactgttttttctatgaaatccgcttcgatggcaccggtccgtttcagcagtttggacgtgatatcgcggataccacggatgcgaccctgaagtgggagccgtccactgaaaaaatgtacgtcgaggacggtgtgctgaagggtgacgttgaaatggcgctgctccttggtggctccggcacgtcatactggaaaggttctatgtggctgtcttacttcatcgccagctttcgtctgcattatagatgcgattttaaaactacgtacaaagcacgcagctacaccccgggcgactccagcagcggctggaccgcacatgaagtggatcatcgtattgagatcttaagtcacattgtggacgaaccgggcggtgcgagcggcaaggtgcgtctgtatgagcacgcggaagcgccggcgccggctccgggttttagcgcattggaaccgctggtagacctgccgggcggcagcggtggtggtgcgagcgggaagaccttcccgcctacagaaccgaaaaaggacaaaaagggtcatcaccaccaccaccacaagaagtaa

SEQ ID NO: 111 - DNA, 1074 bp, Betacoronavirus SARS-CoV-2
atggtaaattacatactaggagtttatcacaagaacaacaaaagctggatggagagcgaattccgcgtttatccggcaccggcgcctatgaaaatcaagctgcgtatggaaggtgcagttaatggtcataaattcgtgattgagggtgaaggcattggcaaaccaggtggttcaggtgaggctgcgaagttcaactgctattttccgctgcagagctacggctttcagccgacgggtacgcagaccctggatctgacggtcgaggaaccgttgcaatcgtacggtttccaaccgacctatgacatcctgaccccggcgttccagtatggcaaccgcgcgttcaccaaatacccggaagccggtaacggtggcgatgccgccctcgctctgttactgttagatggctacagctgggaacgtagcatgacgtacggccgttcctacctgaccccgggtgacagcagctccggtggcgcgtcgggtggtatctgcattgcgaccagtgatattacgatggagggtgactgttttttttatgagatccgctttgatggtaccgcggaccagttgaccccaacttggcgtgtgacccttaagtgggagccgagcaccgaaaaaatgtatgtagaggatggggtgctgaagggcgacgttgaaatggcattgttgttgtttatctataataaaatcgtggacgaaccgggtggcagcggtacttcttactggaaaggttcccattatcgttgcgacttcaaaaccacctacaaagcaaagaacccgttgctatacgatgcgaattaccatgaagttgaccatcgtattgaaattctgagccacggcggctctggaggcgaggctgctaagggtagacctcaaggtctgccgaataacaccgcatccaaagttcgtctgtacgagcacgcggaggcgctggcggagatcctgcaaaaaaacctgatccgccagggtacagattataagcattggccgcaaattgcgggcggctccggtggcggcaagatcgccgactacaattacaagctgggccatcaccaccaccaccacaagaagtaa

SEQ ID NO: 112 - DNA, 1119 bp, Betacoronavirus SARS-CoV-2
atggtaaattatatatttaactgttacttcccgctgcagagctacggcttccagccgaccaatggtgtgatgaaaatcaaactgcgtatggaaggcgcagttcgtccgcagggtctgccgaataacaccgcaagcggcggttccggtggcgaggcggcgaagcataagttcgtgattgaaggtgagggtattggtaagccggaagctgcgaaagaagctgctaagggctctggcttcatctataacaaaatcgttgatgagccgggtgcgggtacgcagaccctggatctgactgtagaggaagcggaccaactgaccccgacctggcgtgtgggctatgacatcttgactccggcgtttcaatatggtaatagagcattcaccaaatacccggagggcaagatcgccgactacaactacaagttgggtggctacagctgggaacgctctatgacctacgaagatcaaggtatttgtattgcgaccagcgatatcaccatggagggtgactgctttttttatgaaatccgctttgatggtacatacattcgcgtgggtgcgcgtaaaagcgctccgctgattgagctgacgctgaaatgggagccgagcaccgaaaaaatgtatgtcgagatcgtggacgaaccgggagggtccggtggtgttctgaagggcgacgttgagatggcactgttgttgaagaacccgttattgtacgatgcaaattacgggggttcgggcacctcctattggaaaggtcattatcgctgcgatttcaaaaccacctataaagcgaagacctttccgccgacggagcctaaaaaagataaaaagcacgaagttgaccatcgtattgagatcctgagccatgaagcggctaagggcggtgccagcggccgtagctacctcactccgggtgacagctctagcaaagttcgtctgtatgagcacgcagaagccccggcgccagcgccaggctcgtccggtgtcgtgaacccggtcatggagccgatttacgacggcggctccggcggtgcgccaggccagacgggaaagatcgccgactataactacaagcttggtgcgtcaggcaaacaccatcaccaccaccacaagaagtaa

SEQ ID NO: 113 - DNA, 975 bp, Betacoronavirus SARS-CoV-2
atggtcaattatatcgcgctcccacaacgccaaaagaagcagcagaccgtgacgctgttgccggcaccggcacccatgaagattaagttacgtatggaaggagcggtcaatgggcacaagtttgtcatcgagggcgaaggaatcgggaaaccttacgaagggacccaaacattagatctgaccgttgaagagggaagcaagagcccaatccaatacattgattacgatattcttacgcctgcatttcagtacggcaatcgtgctttcacaaagtaccctgaagacttcttggagtatcacgacgtgcgcgtagttttagacttcgggtattcttgggaacgtagtatgacgtatggtggagctagcggtggtatcaatgcaagtgttgtcaacatccagggaatctgcatcgccacctcggatatcactatggaaggcgggtccggggaagctgcgaagcaattcgccccctcggctagtgccttcttctgcttcttctacgaaatccgttttgacggtaccatgtatagcttcgtaagcgaagaaacgggcacgctgattgtcaatactctgaagtgggagccgtctacggaaaagatgtatgtagaggacggcgtactgaagggcgacgtcgagatggcgcttctgttagaaggcggcggtcattaccgttgcgattttaagacaacgtataaggcaccatccggtacttggttaacttacacaggccacgaggtcgaccatcgtatcgagatcttatcacatggcggttccggcggcgaagcggccaaggggcagttcgcgcctagtgcatcggctttcttcaaggttcgcctctatgaacacgccgaggcaggcggtgcatctggagagctggacaaatatggcggttcgggcggcggtatgtacagtttcgtatcggaagagacaggaactttaattgtgaatcaccatcaccaccaccataagaagtaa

SEQ ID NO: 114 - DNA, 996 bp, Betacoronavirus SARS-CoV-2
atggtgaattacatcccgctccaatcatacggatttcagccaacgaatggggtcgggtaccctgcgccagcacccgcaatgaaaatcaagcttcgtatggaaggtgcagttaatggacacaagttcgtgatcgagggcgagggcattggcaagccctatgaagggacccaaactttggacttgacggtagaagagggtatctatcagaccagtaattttcgtgtctacgacatcttgactccggctttccaatacgggaaccgcgcgttcactaagtaccctgagaaggcgtacaatgtgacccaagcattcgggcgtcgtggccctgaaggctatagctgggaacgttctatgacgtacgaggatcaaggtatctgtattgcgactagcgatattacaatggaaggcaccaatacatctaatcaagtggcggtgggtggaagcggtgaggcagcaaagtgcttcttttatgagatccgcttcgatggtactaacccggtcctgccctttaacgatggagtttatttcgcctcaaccacattgaaatgggagccctcaactgaaaagatgtatgttgaagacggcgttttgaagggtgacgtagagatggcacttctgctgtacaactacaagcttcccgatgacttcactggcggcagcggtacaagttattggaagggttcacattatcgctgtgatttcaagacaacctataaagcaatgttccatctggtagactttcaagttacgattgctgagatccttcatgaggttgatcaccgtatcgaaatcctttctcacggcggaagcggcggagaggcggccaagggtatgaaggacttgagccctcgctggtatttcaaggtccgcttatacgagcacgccgaggccgacgctgcccttgcgctcctgttattagacggcggaagcggtggtggcatgaaggatttatcccctcgctggtacttccatcaccatcaccaccacaagaagtga

SEQ ID NO: 115 - DNA, 1059 bp, Betacoronavirus SARS-CoV-2
atggtgaattatatcttaggggtgtaccacaagaataacaagtcatggatggagtcggaatttcgtgtgtacccggccccggctcccatgaagatcaagttacgcatggaaggcgccgttaacggccacaaattcgtgatcgagggcgaaggaatcggtaagccaggtggctcgggtgaagccgccaagttcatttacaataagatcgttgatgaaccaggaacacaaactttagatcttaccgtagaggagaagaacccgcttctttacgacgccaattactatgacattttaacgccggccttccaatacggcaatcgcgcttttacgaagtatccggaagcgggtaacggcggcgatgcagccttagcattattattactggacggttattcgtgggagcgttcaatgacgtatggccgtagttacttgacccccggagacagtagctctggtggtgccagcggcgggatctgtattgcaactagtgatatcaccatggaaggcgactgcttcttctatgagattcgtttcgatgggaccgccgatcaactgacccctacctggcgtgtaacattgaagtgggagccaagcactgagaagatgtacgtggaagacggcgttctgaagggcgacgtggagatggccttgttattattcatctacaataagatcgtggacgagccgggtggtagcggtacatcctactggaagggttcgcattaccgttgtgatttcaagacaacctataaggctaagaatccgttattgtatgacgcaaattaccatgaggtggaccaccgtatcgaaatcctttcccatggcggaagcggtggtgaggcggcgaagggtcgcccgcagggactgccgaataatacggcgtctaaggttcgcttatacgaacacgctgaggccctcgccgagatcctgcaaaagaacttaatccgccaaggaaccgactataagcactggccgcaaatcgccggtggctccggtggtggtaagatcgcagactacaactataagctcggtcatcatcatcatcaccataagaaataa

SEQ ID NO: 116 - protein, 346 aa, Betacoronavirus SARS-CoV-2
MVNYLGAHAFERDISTEIYQAGSTMKIKLRMEGAVNGHKFVIEGEGIGKPYEGTQTLDLTVEEGSSGEAAKEAAKGSTPCNGVEGFNCYFYDILTPAFQYGNRAFTKYPEDIPDYFKQAFPEGYSWERSMTYEDQGICIATSDITMEGDCFFYEIRFDGTNSNNLDSKVGGNYNYTLKWEPSTEKMYVEDGVLKGDVEMALLLFERDISTEIYQAGSTGGSGTSYWKGSHYRCDFKTTYKAGSTPCNGVEGFNCYFHEVDHRIEILSHYFPLQSYGFQPTNGVGSSGEAAKEAAKKVRLYEHAEAYFPLQSYGFQPTNGVGGSGGGASGNSNNLDSKVGGNYNYLE

SEQ ID NO: 117 - protein, 352 aa, Betacoronavirus SARS-CoV-2
MVNYLGAHAFERDISTEIYQAGSTMKIKLRMEGAVNGHKFVIEGEGIGKPYEGTQTLDLTVEEGSSGEAAKEAAKGSTPCNGVEGFNCYFYDILTPAFQYGNRAFTKYPEDIPDYFKQAFPEGYSWERSMTYEDQGICIATSDITMEGDCFFYEIRFDGTNSNNLDSKVGGNYNYTLKWEPSTEKMYVEDGVLKGDVEMALLLFERDISTEIYQAGSTGGSGTSYWKGSHYRCDFKTTYKAGSTPCNGVEGFNCYFHEVDHRIEILSHYFPLQSYGFQPTNGVGSSGEAAKEAAKKVRLYEHAEAYFPLQSYGFQPTNGVGGSGGGASGNSNNLDSKVGGNYNYLEHHHHHH

SEQ ID NO: 118 - protein, 375 aa, Betacoronavirus SARS-CoV-2
MVNYIGSSGVVNPVMGGSGGPAPAPMKIKLRMEGAVNAPRITFGGPSDSTGGGSGEAAKTSYWKGSHKFVIEGEGIGKPYEGTQTLDLTVEEGSSGVVNPVMYDILTPAFQYGNRAFTKYPEYIRVGARKSAPLIELGYSWERSMTYGSLIDLQELGKYEQYIEAAAKEAAAKGGASGICIATSDITMEGDCFFYEIRFDGTGPFQQFGRDIADTTDATLKWEPSTEKMYVEDGVLKGDVEMALLLGGSGTSYWKGSMWLSYFIASFRLHYRCDFKTTYKARSYTPGDSSSGWTAHEVDHRIEILSHIVDEPGGASGKVRLYEHAEAPAPAPGFSALEPLVDLPGGSGGGASGKTFPPTEPKKDKKGHHHHHHKK

SEQ ID NO: 119 - protein, 357 aa, Artificial Sequence
  SYNTHETIC CONSTRUCT
  misc_feature: Thermal Green Protein, Betacoronavirus SARS-CoV-2
MVNYILGVYHKNNKSWMESEFRVYPAPAPMKIKLRMEGAVNGHKFVIEGEGIGKPGGSGEAAKFNCYFPLQSYGFQPTGTQTLDLTVEEPLQSYGFQPTYDILTPAFQYGNRAFTKYPEAGNGGDAALALLLLDGYSWERSMTYGRSYLTPGDSSSGGASGGICIATSDITMEGDCFFYEIRFDGTADQLTPTWRVTLKWEPSTEKMYVEDGVLKGDVEMALLLFIYNKIVDEPGGSGTSYWKGSHYRCDFKTTYKAKNPLLYDANYHEVDHRIEILSHGGSGGEAAKGRPQGLPNNTASKVRLYEHAEALAEILQKNLIRQGTDYKHWPQIAGGSGGGKIADYNYKLGHHHHHHKK

SEQ ID NO: 120 - protein, 372 aa, Artificial Sequence
  SYNTHETIC CONSTRUCT
  MISC_FEATURE: Betacoronavirus SARS-CoV-2
MVNYIFNCYFPLQSYGFQPTNGVMKIKLRMEGAVRPQGLPNNTASGGSGGEAAKHKFVIEGEGIGKPEAAKEAAKGSGFIYNKIVDEPGAGTQTLDLTVEEADQLTPTWRVGYDILTPAFQYGNRAFTKYPEGKIADYNYKLGGYSWERSMTYEDQGICIATSDITMEGDCFFYEIRFDGTYIRVGARKSAPLIELTLKWEPSTEKMYVEIVDEPGGSGGVLKGDVEMALLLKNPLLYDANYGGSGTSYWKGHYRCDFKTTYKAKTFPPTEPKKDKKHEVDHRIEILSHEAAKGGASGRSYLTPGDSSSKVRLYEHAEAPAPAPGSSGVVNPVMEPIYDGGSGGAPGQTGKIADYNYKLGASGKHHHHHHKK

SEQ ID NO: 121 - protein, 324 aa, Artificial Sequence
  SYNTHETIC CONSTRUCT
  MISC_FEATURE: Thermal Green Protein, Betacoronavirus SARS-CoV-2
MVNYIALPQRQKKQQTVTLLPAPAPMKIKLRMEGAVNGHKFVIEGEGIGKPYEGTQTLDLTVEEGSKSPIQYIDYDILTPAFQYGNRAFTKYPEDFLEYHDVRVVLDFGYSWERSMTYGGASGGINASVVNIQGICIATSDITMEGGSGEAAKQFAPSASAFFCFFYEIRFDGTMYSFVSEETGTLIVNTLKWEPSTEKMYVEDGVLKGDVEMALLLEGGGHYRCDFKTTYKAPSGTWLTYTGHEVDHRIEILSHGGSGGEAAKGQFAPSASAFFKVRLYEHAEAGGASGELDKYGGSGGGMYSFVSEETGTLIVNHHHHHHKK

SEQ ID NO: 122 - protein, 331 aa, artificial
  SYNTHETIC CONSTRUCT
  MISC_FEATURE: Betacoronavirus SARS-CoV-2
MVNYIPLQSYGFQPTNGVGYPAPAPAMKIKLRMEGAVNGHKFVIEGEGIGKPYEGTQTLDLTVEEGIYQTSNFRVYDILTPAFQYGNRAFTKYPEKAYNVTQAFGRRGPEGYSWERSMTYEDQGICIATSDITMEGTNTSNQVAVGGSGEAAKCFFYEIRFDGTNPVLPFNDGVYFASTTLKWEPSTEKMYVEDGVLKGDVEMALLLYNYKLPDDFTGGSGTSYWKGSHYRCDFKTTYKAMFHLVDFQVTIAEILHEVDHRIEILSHGGSGGEAAKGMKDLSPRWYFKVRLYEHAEADAALALLLLDGGSGGGMKDLSPRWYFHHHHHHKK

SEQ ID NO: 123 - protein, 352 aa, Artificial Sequence
  SYNTHETIC CONSTRUCT
  MISC_FEATURE: Betacoronavirus SARS-CoV-2
MVNYILGVYHKNNKSWMESEFRVYPAPAPMKIKLRMEGAVNGHKFVIEGEGIGKPGGSGEAAKFIYNKIVDEPGTQTLDLTVEEKNPLLYDANYYDILTPAFQYGNRAFTKYPEAGNGGDAALALLLLDGYSWERSMTYGRSYLTPGDSSSGGASGGICIATSDITMEGDCFFYEIRFDGTADQLTPTWRVTLKWEPSTEKMYVEDGVLKGDVEMALLLFIYNKIVDEPGGSGTSYWKGSHYRCDFKTTYKAKNPLLYDANYHEVDHRIEILSHGGSGGEAAKGRPQGLPNNTASKVRLYEHAEALAEILQKNLIRQGTDYKHWPQIAGGSGGGKIADYNYKLGHHHHHHKK

SEQ ID NO: 124 - protein, 15 aa, Betacoronavirus SARS-CoV-2
FERDISTEIYQAGST

SEQ ID NO: 125 - protein, 15 aa, Betacoronavirus SARS-CoV-2
GSTPCNGVEGFNCYF

SEQ ID NO: 126 - protein, 15 aa, Betacoronavirus SARS-CoV-2
NSNNLDSKVGGNYNY

SEQ ID NO: 127 - protein, 10 aa, Betacoronavirus SARS-CoV-2
GSSGVVNPVM

SEQ ID NO: 128 - protein, 16 aa, Betacoronavirus SARS-CoV-2
NAPRITFGGPSDSTGS

SEQ ID NO: 129 - protein, 15 aa, Betacoronavirus SARS-CoV-2
YIRVGARKSAPLIEL

SEQ ID NO: 130 - protein, 15 aa, Betacoronavirus SARS-CoV-2
SLIDLQELGKYEQYI

SEQ ID NO: 131 - protein, 15 aa, Betacoronavirus SARS-CoV-2
PFQQFGRDIADTTDA

SEQ ID NO: 132 - protein, 12 aa, Betacoronavirus SARS-CoV-2
MWLSYFIASFRL

SEQ ID NO: 133 - protein, 5 aa, Betacoronavirus SARS-CoV-2
IVDEP

SEQ ID NO: 134 - protein, 12 aa, Betacoronavirus SARS-CoV-2
GFSALEPLVDLP

SEQ ID NO: 135 - protein, 13 aa, Betacoronavirus SARS-CoV-2
KTFPPTEPKKDKK

SEQ ID NO: 136 - protein, 19 aa, Betacoronavirus SARS-CoV-2
LGVYHKNNKSWMESEFRVY

SEQ ID NO: 137 - protein, 15 aa, Betacoronavirus SARS-CoV-2
FNCYFPLQSYGFQPT

SEQ ID NO: 138 - protein, 10 aa, Betacoronavirus SARS-CoV-2
PLQSYGFQPT

SEQ ID NO: 139 - protein, 15 aa, Betacoronavirus SARS-CoV-2
AGNGGDAALALLLLD

SEQ ID NO: 140 - protein, 11 aa, Betacoronavirus SARS-CoV-2
RSYLTPGDSSS

SEQ ID NO: 141 - protein, 10 aa, Betacoronavirus SARS-CoV-2
ADQLTPTWRV

SEQ ID NO: 142 - protein, 10 aa, Betacoronavirus SARS-CoV-2
FIYNKIVDEP

SEQ ID NO: 143 - protein, 10 aa, Betacoronavirus SARS-CoV-2
KNPLLYDANY

SEQ ID NO: 144 - protein, 11 aa, Betacoronavirus SARS-CoV-2
RPQGLPNNTAS

SEQ ID NO: 145 - protein, 23 aa, Betacoronavirus SARS-CoV-2
LAEILQKNLIRQGTDYKHWPQIA

SEQ ID NO: 146 - protein, 10 aa, Betacoronavirus SARS-CoV-2
GKIADYNYKL

SEQ ID NO: 147 - protein, 15 aa, Betacoronavirus SARS-CoV-2
GSSGVVNPVMEPIYD

SEQ ID NO: 148 - protein, 15 aa, Betacoronavirus SARS-CoV-2
APGQTGKIADYNYKL

SEQ ID NO: 149 - protein, 15 aa, Betacoronavirus SARS-CoV-2
ALPQRQKKQQTVTLL

SEQ ID NO: 150 - protein, 10 aa, Betacoronavirus SARS-CoV-2
GSKSPIQYID

SEQ ID NO: 151 - protein, 14 aa, Betacoronavirus SARS-CoV-2
DFLEYHDVRVVLDF

SEQ ID NO: 152 - protein, 10 aa, Betacoronavirus SARS-CoV-2
GINASVVNIQ

SEQ ID NO: 153 - protein, 10 aa, Betacoronavirus SARS-CoV-2
QFAPSASAFF

SEQ ID NO: 154 - protein, 10 aa, Betacoronavirus SARS-CoV-2
PSGTWLTYTG

SEQ ID NO: 155 - protein, 5 aa, Betacoronavirus SARS-CoV-2
ELDKY

SEQ ID NO: 156 - protein, 10 aa, Betacoronavirus SARS-CoV-2
GIYQTSNFRV

SEQ ID NO: 157 - protein, 15 aa, Betacoronavirus SARS-CoV-2
KAYNVTQAFGRRGPE

SEQ ID NO: 158 - protein, 10 aa, Betacoronavirus SARS-CoV-2
GTNTSNQVAV

SEQ ID NO: 159 - protein, 15 aa, Betacoronavirus SARS-CoV-2
NPVLPFNDGVYFAST

SEQ ID NO: 160 - protein, 10 aa, Betacoronavirus SARS-CoV-2
YNYKLPDDFT

SEQ ID NO: 161 - protein, 15 aa, Betacoronavirus SARS-CoV-2
MFHLVDFQVTIAEIL

SEQ ID NO: 162 - protein, 10 aa, Betacoronavirus SARS-CoV-2
MKDLSPRWYF

SEQ ID NO: 163 - protein, 10 aa, Betacoronavirus SARS-CoV-2
DAALALLLLD